(19) United States
(12) Patent Application Publication
(10) Pub. No.: US 2023/0382886 A1
(43) Pub. Date: Nov. 30, 2023

(54) CONJUGATED TLR7 AND NOD2 AGONISTS

(71) Applicant: UNIVERZA V LJUBLJANI, Ljubljana (SI)

(72) Inventors: Ziga Jakopin, Ljubljana (SI); Samo Guzelj, Dobrova (SI)

(21) Appl. No.: 18/032,612

(22) PCT Filed: Oct. 20, 2021
(86) PCT No.: PCT/EP2021/079141

§ 371 Date: Apr. 19, 2023

(30) Foreign Application Priority Data
Oct. 21, 2020 (LU) LU102145

(51) Int. Cl. C07D 401/04; A61K 47/55; A61P 37/04

(52) U.S. Cl. CPC C07D 401/04; A61K 47/55; A61P 37/04

(57) ABSTRACT

This invention provides covalent conjugates of TLR7 and NOD2 agonists, processes for preparing such compounds and the use of such compounds in medicine.

### FIELD OF THE INVENTION

The present invention provides covalent conjugates of TLR7 and NOD2 agonists, processes for their preparation and their use in therapeutic applications.

### BACKGROUND OF THE INVENTION

The innate immune system, the cornerstone of the body's ability to combat infection, is comprised of APCs, in particular dendritic cells (DCs), which contain a series of pattern recognition receptors (PRRs), including Toll-like receptors (TLRs), nucleotide-binding oligomerization domain (NOD)-like receptors (NLRs), retinoic acid-inducible gene-1-like receptors (RLRs), C-type lectin receptors (CLRs) and stimulator of interferon genes (STING). They are activated by a diverse set of immunostimulatory molecules dubbed pathogen-associated molecular patterns (PAMPs), which engage DCs to more effectively uptake and present antigens via inflammatory cytokines, interferons (IFNs) and co-stimulatory molecules and provide indispensable initial signals that determine the type of adaptive response, such as cellular (Th1) or humoral (Th2) response, as well as its magnitude and durability (Gutjahr et al. 2016).

NOD2 is an intracellular PRR of the NLR family, widely expressed in immune cells, that recognizes fragments of bacterial peptidoglycan resembling muramyl dipeptide (MDP) (Jakopin 2014). Stimulation of NOD2 activates nuclear factor κB (NF-κB) and mitogen activated protein kinase (MAPK) signalling pathways, which results in pro-inflammatory cytokine production, type I IFN secretion and expression of co-stimulatory molecules. Importantly, NOD2 agonists trigger the maturation and activation of DCs (Asano et al. 2010, Vidal et al. 2001). NOD2 agonists have also been shown to induce autophagy (Cooney et al. 2010, Travassos et al. 2010), which is pivotal for the induction of efficient antigen cross-presentation and promoting innate cytokine production in DCs (Morris et al. 2011). Hence, NOD activation albeit not the most potent innate immune inducer by itself is indispensable to shape adaptive immune responses (Kobayashi et al. 2005). Notably, NLR expression in B cells is required for optimal humoral response. The involvement of NODs in numerous mechanisms of immunity makes them excellent targets for vaccine adjuvants; their use in cancer vaccines was recently highlighted (Nabergoj et al. 2019). Specifically, engagement of NOD2 proved to be essential for antigen-specific mucosal and systemic responses of mucosal vaccines (Bumgardner et al. 2018, Jackson et al. 2012). Previous work established the essential structural requirements of NOD2 agonists, which culminated in the discovery of low nanomolar NOD2 agonists, amenable to covalent coupling to other molecules (Jakopin et al. 2012, Gobec et al. 2016, 2018).

TLR7 is an intracellular endosomal receptor, which detects single-stranded RNA and subsequently induces signalling pathways mediated via IRF7 and NF-κB. In humans, TLR7 is mainly expressed in plasmacytoid DCs and its activation induces the production of IFNa and IL-12, which prime neighbouring NK, T cells and DCs. IFN-α enhances the cytotoxic potential of NK cells, while IL-12 augments IFN-γ secretion from NK cells. TLR7 also contributes to maturation and differentiation of DCs, generating DCs with better co-stimulatory abilities and enhanced antigen presenting abilities (Kobold et al. 2014). Several small molecule TLR7 agonists have been identified, including imidazoquinolines, purines and 3-deazapurines (Hemmi et al. 2002, Lee et al. 2003, Jones et al. 2011). Imiquimod, the prototypical member of the of the imidazoquinoline class, is effective against genital warts, basal cell carcinoma and actinic keratosis when applied as a topical cream. In the purine class, 2-substituted 8-hydroxyadenines were found to be potent TLR7 agonists (Kurimoto et al. 2004, 2010) with amenability for covalent conjugation to other molecules (Akinbobuyi et al. 2016). Additional disclosures of TLR7 agonists with a purine-like structure and their uses in medicine include: Tran et al. 2011, Nakamura et al. 2013, Akinbobuyi et al. 2015, WO2006/117670, WO2007/024707, WO2008/004948, WO2010/093436, WO2010/018134, WO2011/134668, WO2012/038058, WO2019/209811, WO2019/035969, WO2019/036023, WO2019/035971, WO2019/197598.

Simultaneous activation of distinct PRRs (cross-activation) permits signal amplification, enables integration of stimuli and facilitates fine-tuning (directs polarization of response) (Thaiss et al. 2016). Cross-talk between multiple PRRs can be both quantitatively and qualitatively different than the contribution of each individual pathway. Effective live attenuated vaccines engage the immune system through a synergistic stimulation of several independent PRRs to achieve a systemic response. Further, covalently linked chimeric dual/triple PRR agonists demonstrated synergistic immunostimulatory activity over their unconjugated mixtures (Tom et al. 2019).

Numerous studies reported that conjugating multiple PRR agonists via ester and amide linkages impacts their potency, toxicity or pharmacokinetics. TLR agonists have been at the forefront of adjuvant development because they are well characterized and can elicit a strong Th1 response, which many vaccines lack. Dual TLR agonists, including TLR2/TLR9 (Mancini et al. 2014), TLR4/9 (Madan-Lala et al. 2017), TLR2/TLR7 (Gutjahr et al. 2017) have been reported. A chimeric ligand constructed by linking TLR2 and TLR7 agonists elicited potent and balanced cellular and humoral responses (Gutjahr et al. 2017). This conjugate also proved to be much safer than equal amounts of individual agonists. Namely, adjuvants consisting of mixtures of unconjugated agonists can easily diffuse through the immune system. Similarly, a dual NOD2/TLR2 agonist increased the immunogenicity of HIV-1 subunit vaccine (Pavot et al. 2014).

In view of the great therapeutic potential of conjugated NLR/TLR agonists, and despite the work that has already been done, there is an ongoing need for new potent compounds capable of eliciting responses from the aforementioned receptors. Thus, this specification discloses novel conjugated compounds that function as both TLR7 and NOD2 agonists, the processes for their preparation and their uses in medicine.

### SUMMARY OF THE INVENTION

In accordance with the purpose of this invention, as embodied and described herein, the present application discloses novel compounds that are synthetic TLR7 agonists covalently conjugated to synthetic NOD2 agonists. Further provided are the processes for preparation of such compounds and the uses of such compounds in medicine.

In a first aspect, the present invention provides a compound of formula I:

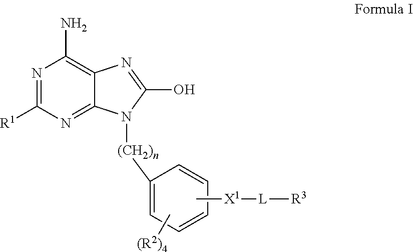

or a pharmaceutically acceptable salt, racemate, diastereomer, enantiomer, ester or prodrug thereof, wherein:
R¹ is H, halogen, OH, SH, CF₃, C₁-C₆ alkyl, C₃-C₁₀ cycloalkyl, C₆-C₁₀ aryl, C₅-C₉ heterocyclyl, C₁-C₆ alkoxy, C₁-C₆ alkoxy C₁-C₆ alkyl, C₁-C₆ alkoxy-C₁-C₆ alkoxy, C₁-C₆ alkoxy-(C₁-C₆ alkyl)S—, (C₁-C₆ alkyl)SO₂NH—, (C₁-C₆ alkyl)C(═O)O—, (C₁-C₆ alkyl)OC(═O)—, (C₁-C₆ alkyl)C(═O)—, (C₁-C₆ alkyl)C(═O)NH—, R^{a}R^{b}N— or R^{a}R^{b}N(C═O)—, wherein alkyl, alkoxy, cycloalkyl, aryl and heterocyclyl may be optionally substituted;
R² is independently for each occurrence selected from H, halogen, OH, CHF₂, CF₃, CH₂CF₂, carboxy, CN, NO₂, C₁-C₆ alkyl, C₃-C₁₀ cycloalkyl, C₁-C₆ alkoxy, (C₁-C₆ alkyl)C(═O)—, (C₆-C₁₀ aryl)C(═O)—, (C₁-C₆ alkyl)S—, (C₁-C₆ alkyl)C(═O)O—, (C₁-C₆ alkyl)OC(═O)—, (C₁-C₆ alkyl)C(═O)NH—, (C₁-C₆ alkyl)SO₂NH—, R^{a}R^{b}N— and R^{a}R^{b}N(C═O)—, wherein alkyl, alkoxy, aryl and cycloalkyl may be optionally substituted;
R^{a} and R^{b} are independently from each other selected from H, C₁-C₆ alkyl, C₃-C₁₀ cycloalkyl, C₆-C₁₀ aryl and C₅-C₉ heterocyclyl or R^{a} and R^{b} may together with the nitrogen atom form a C₅-C₆ heterocycle, wherein alkyl, cycloalkyl, aryl and heterocyclyl may be optionally substituted; preferably R^{a} and R^{b} are independently from each other selected from H, C₃-C₁₀ cycloalkyl, C₆-C₁₀ aryl and C₅-C₉ heterocyclyl or R^{a} and R^{b} may together with the nitrogen atom form a C₅-C₆ heterocycle, wherein alkyl, cycloalkyl, aryl and heterocyclyl may be optionally substituted;
X¹ is a single bond, —O—, —S—, —NR_{c}-, -C(═O)— or —SO₂—;
R^{c} is H, C₁-C₆ alkyl or C₃-C₁₀ cycloalkyl;
L is a linking group;
n is 0, 1, 2, 3 or 4; and
R³ is a NOD2 agonist selected from the group consisting of:

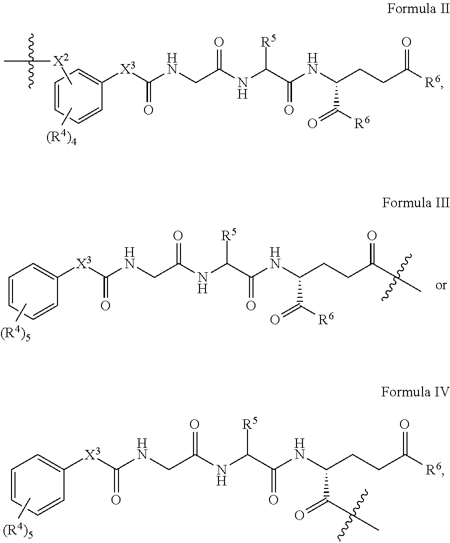

wherein:
X² is a single bond, —O—, —S—, —NR_{d}-, —C(═O)— or —SO₂—;
R^{d} is H, C₁-C₆ alkyl or C₃-C₁₀ cycloalkyl;
X³ is —(CH₂)₂—, —(CH═CH)— or cyclopropylene;
R⁴ is independently for each occurrence selected from H, halogen, OH, CHF₂, CF₃, CH₂CF₂, carboxy, CN, NO₂, C₁-C₆ alkyl, C₁-C₆ alkoxy, C₃-C₁₀ cycloalkyl, (C₁-C₆ alkyl)C(═O)—, (C₁-C₆ aryl)C(═O)—, (C₁-C₆ alkyl)S—, (C₁-C₆ alkyl)C(═O)O—, (C₁-C₆ alkyl)OC(═O)—, (C₁-C₆ alkyl)C(═O)NH—, (C₁-C₆ alkyl)SO₂NH—, R^{e}R^{f}N— and R^{e}R^{f}N(C═O)—, wherein alkyl, alkoxy, aryl and cycloalkyl may be optionally substituted;
R^{e} and R^{f} are independently from each other selected from H, C₁-C₆ alkyl, C₃-C₁₀ cycloalkyl, C₆-C₁₀ aryl and C₅-C₉ heterocyclyl or R^{e} and R^{f} may together with the nitrogen atom form a C₅-C₆ heterocycle;
R⁵ is C₁-C₆ alkyl or a specific side chain of an amino acid;
R⁶ is independently for each occurrence selected from OH, C₁-C₁₈ alkoxy, (C₂-C₁₈ alkenyl)O—, (C₃-C₁₀ cycloalkyl)O—, (C₆-C₁₀ aryl)O—, (C₅-C₉ heterocyclyl)O—and R^{g}R^{h}N—, wherein alkoxy, alkenyl, cycloalkyl, aryl or heterocyclyl may be optionally substituted; and
R^{g} and R^{h} are independently from each other selected from H, C₁-C₁₈ alkyl, C₂-C₁₈ alkenyl, C₃-C₁₀ cycloalkyl, C₆-C₁₀ aryl and C₅-C₉ heterocyclyl or R^{g} and R^{h} may together with the nitrogen atom form a C₅-C₆ heterocycle wherein alkyl, alkenyl, cycloalkyl, aryl and heterocyclyl may be optionally substituted.

In further aspect, the invention provides a process and intermediates for the preparation of compounds of Formula I or a pharmaceutically acceptable salt, racemate, diastereomer, enantiomer, ester or prodrug thereof.

In a further aspect, the present invention provides a compound of Formula I or a pharmaceutically acceptable salt, racemate, diastereomer, enantiomer, ester or prodrug thereof, for use in therapy.

In a further aspect, the invention provides a pharmaceutical composition comprising a therapeutically effective amount of a compound of Formula I or a pharmaceutically acceptable salt, racemate, diastereomer, enantiomer, ester or prodrug thereof, and a pharmaceutically acceptable excipient or carrier.

In a further aspect, the invention provides the use of a compound of Formula I or a pharmaceutically acceptable salt, racemate, diastereomer, enantiomer, ester or prodrug thereof, in treatment of conditions for which agonism of TLR7 and NOD2 receptors is beneficial.

In a further aspect, the invention provides the use of a compound of Formula I or a pharmaceutically acceptable salt, racemate, diastereomer, enantiomer, ester or prodrug thereof in the manufacture of a medicament.

In a further aspect, the invention provides a method for treating a subject in need thereof, comprising administering to the subject a therapeutically effective amount of a compound of Formula I or a pharmaceutically acceptable salt, racemate, diastereomer, enantiomer, ester or prodrug thereof, or a pharmaceutical composition of the present invention.

In a further aspect, the invention provides a vaccine comprising a compound of Formula I or a pharmaceutically acceptable salt, racemate, diastereomer, enantiomer, ester or prodrug thereof.

The present invention can be further summarized by the following items:

1. A compound having a structure according to Formula I:

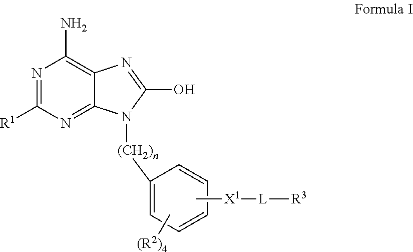

or a pharmaceutically acceptable salt, racemate, diastereomer, enantiomer, ester or prodrug thereof, wherein:
R¹ is H, halogen, OH, SH, CF₃, C₁-C₆ alkyl, C₃-C₁₀ cycloalkyl, C₆-C₁₀ aryl, C₅-C₉ heterocyclyl, C₁-C₆ alkoxy, C₁-C₆ alkoxy C₁-C₆ alkyl, C₁-C₆ alkoxy-C₁-C₆ alkoxy, C₁-C₆ alkoxy-(C₁-C₆ alkyl)S—, (C₁-C₆ alkyl)SO₂NH—, (C₁-C₆ alkyl)C(═O)O—, (C₁-C₆ alkyl)OC(═O)—, (C₁-C₆ alkyl)C(═O)—, (C₁-C₆ alkyl)C(═O)NH—, R^{a}R^{b}N— or R^{a}R^{b}N(C═O)—, wherein alkyl, alkoxy, cycloalkyl, aryl and heterocyclyl may be optionally substituted;
R² is independently for each occurrence selected from H, halogen, OH, CHF₂, CF₃, CH₂CF₂, carboxy, CN, NO₂, C₁-C₆ alkyl, C₃-C₁₀ cycloalkyl, C₁-C₆ alkoxy, (C₁-C₆ alkyl)C(═O)—, (C₆-C₁₀ aryl)C(═O)—, (C₁-C₆ alkyl)S—, (C₁-C₆ alkyl)C(═O)O—, (C₁-C₆ alkyl)OC(═O)—, (C₁-C₆ alkyl)C(═O)NH—, (C₁-C₆ alkyl)SO₂NH—, R^{a}R^{b}N— and R^{a}R^{b}N(C═O)—, wherein alkyl, alkoxy, aryl and cycloalkyl may be optionally substituted;
R^{a} and R^{b} are independently from each other selected from H, C₁-C₆ alkyl, C₃-C₁₀ cycloalkyl, C₆-C₁₀ aryl and C₅-C₉ heterocyclyl or R^{a} and R^{b} may together with the nitrogen atom form a C₅-C₆ heterocycle, wherein alkyl, cycloalkyl, aryl and heterocyclyl may be optionally substituted; preferably R^{a} and R^{b} are independently from each other selected from H, C₃-C₁₀ cycloalkyl, C₆-C₁₀ aryl and C₅-C₉ heterocyclyl or R^{a} and R^{b} may together with the nitrogen atom form a C₅-C₆ heterocycle, wherein alkyl, cycloalkyl, aryl and heterocyclyl may be optionally substituted;
X¹ is a single bond, —O—, —S—, —NR_{c}-, —C(═O)— or —SO₂—;
R^{c} is H, C₁-C₆ alkyl or C₃-C₁₀ cycloalkyl;
L is a linking group;
n is 0, 1, 2, 3 or 4; and
R³ is a NOD2 agonist selected from the group consisting of:

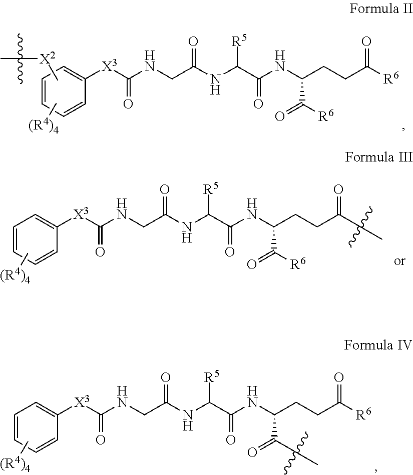

wherein:
X² is a single bond, —O—, —S—, —NR_{d}-, —C(═O)— or —SO₂—;
R^{d} is H, C₁-C₆ alkyl or C₃-C₁₀ cycloalkyl;
X³ is —(CH₂)₂—, —(CH═CH)— or cyclopropylene;
R⁴ is independently for each occurrence selected from H, halogen, OH, CHF₂, CF₃, CH₂CF₂, carboxy, CN, NO₂, C₁-C₆ alkyl, C₁-C₆ alkoxy, C₃-C₁₀ cycloalkyl, (C₁-C₆ alkyl)C(═O)—, (C₁-C₆ aryl)C(═O)—, (C₁-C₆ alkyl)S—, (C₁-C₆ alkyl)C(═O)O—, (C₁-C₆ alkyl)OC(═O)—, (C₁-C₆ alkyl)C(═O)NH—, (C₁-C₆ alkyl)SO₂NH—, R^{e}R^{f}N— and R^{e}R^{f}N(C═O)—, wherein alkyl, alkoxy, aryl and cycloalkyl may be optionally substituted;
R^{e} and R^{f} are independently from each other selected from H, C₁-C₆ alkyl, C₃-C₁₀ cycloalkyl, C₆-C₁₀ aryl and C₅-C₉ heterocyclyl or R^{e} and R^{f} may together with the nitrogen atom form a C₅-C₆ heterocycle;
R⁵ is C₁-C₆ alkyl or a specific side chain of an amino acid;
R⁶ is independently for each occurrence selected from OH, C₁-C₁₈ alkoxy, (C₂-C₁₈ alkenyl)O—, (C₃-C₁₀ cycloalkyl)O—, (C₆-C₁₀ aryl)O—, (C₅-C₉ heterocyclyl)O—and R^{g}R^{h}N—, wherein alkoxy, alkenyl, cycloalkyl, aryl or heterocyclyl may be optionally substituted; and
R^{g} and R^{h} are independently from each other selected from H, C₁-C₁₈ alkyl, C₂-C₁₈ alkenyl, C₃-C₁₀ cycloalkyl, C₆-C₁₀ aryl and C₅-C₉ heterocyclyl or R^{g} and R^{h} may together with the nitrogen atom form a C₅-C₆ heterocycle wherein alkyl, alkenyl, cycloalkyl, aryl and heterocyclyl may be optionally substituted.

2. Compound according to item 1, wherein n is 1.

3. Compound according to item 1 or 2, wherein R¹ is hydrogen, C₁-C₆ alkoxy, C₁-C₆ alkoxy-C₁-C₆ alkoxy, (C₁-C₆ alkyl)NH, C₁-C₆ alkoxy-(C₁-C₆ alkyl)NH, (C₁-C₆ alkyl)S—or CF₃; preferably R¹ is hydrogen, C₁-C₆ alkoxy, C₁-C₆ alkoxy-C₁ C₆ alkoxy, (C₁ C₆ alkyl)S or CF₃.

4. Compound according to any one of items 1 to 3, wherein R¹ is C₁-C₆ alkoxy or C₁-C₆ alkoxy-C₁-C₆ alkoxy.

5. Compound according to any one of items 1 to 3, wherein R¹ is C₁-C₆ alkoxy.

6. Compound according to any one of items 1 to 3, wherein R¹ is C₁-C₆ alkoxy-C₁-C₆ alkoxy.

7. Compound according to any one of items 1 to 3, wherein R¹ is n-BuO—.

8. Compound according to any one of items 1 to 7, wherein R² is independently for each instance selected from hydrogen, halogen and C₁-C₆ alkyl.

9. Compound according to item 8, wherein R² is hydrogen in each instance.

10. Compound according to any one of items 1 to 9, wherein X¹ is —O—, —NH— or —C(═O)—.

11. Compound according to any one of items 1 to 9, wherein X¹ is —C(═O)—.

12. Compound according to any one of items 1 to 11, wherein X¹ is in para position relative to the (CH₂)ₙ group.

13. A compound according to any one of items 1 to 12, wherein L is selected from the group consisting of an amino acid, a peptide, a non-peptidic polymeric linker and a non-polymeric aliphatic linker.

14. Compound according to item 13, wherein L is a non-peptidic polymeric linker or a non-polymeric aliphatic linker.

15. Compound according to item 14, wherein L is a polyethylene glycol chain comprising of 2 to 100 repeating ethylene glycol units.

16. Compound according to item 14, wherein L is a polyethylene glycol chain comprising of 2 to 50 repeating ethylene glycol units.

17. Compound according to item 14, wherein L is a polyethylene glycol chain comprising of 2 to 20 repeating ethylene glycol units.

18. Compound according to item 14, wherein L is a polyethylene glycol chain comprising of 2, 3, 4, 6, 7, 8, 9 or 10 repeating ethylene glycol units.

19. Compound according to item 13, wherein L is a non-polymeric aliphatic linker.

20. Compound according to any one of items 1 to 19, wherein R³ is

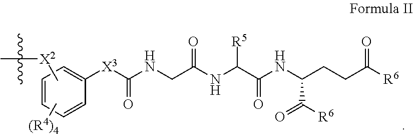

21. Compound according to any one of items 1 to 20, wherein X² is —O—.

22. Compound according to any one of items 1 to 21, wherein X² is—in para position relative to the X³ group.

23. Compound according to any one of items 1 to 19, wherein R³ is

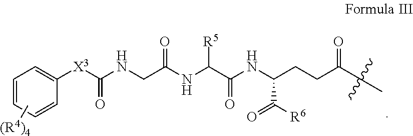

24. Compound according to any one of items 1 to 19, wherein R³ is

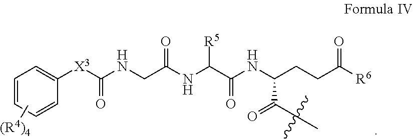

25. Compound according to any one of items 1 to 24, wherein X³ is —CH═CH— or cyclopropylene.

26. Compound according to any one of items 1 to 25, wherein R⁴ is independently for each instance selected from hydrogen, halogen, OH, C₁-C₆ alkyl and C₁-C₆ alkoxy.

27. Compound according to any one of items 1 to 26, wherein R⁴ is independently for each instance selected from hydrogen, halogen, OH, and C₁-C₆ alkoxy.

28. Compound according to any one of items 1 to 27, wherein R⁴ is C₁-C₆ alkoxy in meta position relative to the X³ group, OH in para position relative to the X³ group and H in other instances.

29. Compound according to any one of items 1 to 28, wherein R⁴ is methoxy in meta position relative to the X³ group, OH in para position relative to the X³ group and H in other instances.

30. Compound according to any one of items 1 to 27, wherein R⁴ is fluoro in meta position relative to the X³ group, fluoro in para position relative to the X³ group and H in other instances.

31. Compound according to any one of items 1 to 30, wherein R⁵ is C₁-C₆ alkyl or a specific side chain of a natural amino acid.

32. Compound according to any one of items 1 to 31, wherein R⁵ is a specific side chain of a natural amino acid.

33. Compound according to any one of items 1 to 31, wherein R⁵ is C₁-C₆ alkyl or the specific side chain of valine, alanine, phenylalanine, leucine or isoleucine.

34. Compound according to any one of items 1 to 33, wherein R⁵ is the specific side chain of valine.

35. Compound according to any one of items 1 to 34, wherein R⁶ is independently for each instance selected from OH, NH₂, (C₂-C₁₈ alkenyl)O—, (C₃-C₁₀ cycloalkyl)O— and C₁-C₁₈ alkoxy.

36. Compound according to any one of items 1 to 35, wherein R⁶ is independently for each instance selected from OH, NH₂, (C₂-C₁₀ alkenyl)O—, (C₃-C₁₀ cycloalkyl)O— and C₁-C₁₀ alkoxy.

37. Compound according to any one of items 1 to 36, wherein R⁶ is independently for each instance selected from OH, NH₂, (C₂-C₆ alkenyl)O—, (C₃-C₁₀ cycloalkyl)O— and C₁-C₆ alkoxy.

38. Compound according to any one of items 1 to 35, wherein R⁶ is independently for each instance selected from OH, (C₃-C₁₀ cycloalkyl)O— and C₁-C₁₈ alkoxy.

39. Compound according to any one of items 1 to 38, wherein R⁶ is independently for each instance selected from OH, (C₃-C₅ cycloalkyl)O— and C₁-C₆ alkoxy.

40. Compound according to any one of items 1 to 38, wherein R⁶ is independently for each instance selected from OH and C₁-C₆ alkoxy.

41. Compound according to any one of items 1 to 38, wherein R⁶ is independently for each instance selected from (C₃-C₅ cycloalkyl)O— and C₁-C₆ alkoxy.

42. Compound according to any one of items 1 to 38, wherein R⁶ is independently for each instance selected from OH and (C₃-C₅ cycloalkyl)O—.

43. Compound according to any one of items 1 to 39, wherein R⁶ is OH in each instance.

44. Compound according to any one of items 1 to 39, wherein R⁶ is C₁-C₆ alkoxy in each instance.

45. Compound according to any one of items 1 to 39, wherein R⁶ is ethoxy in each instance.

46. Compound according to any one of items 1 to 39, wherein R⁶ is (C₃-C₁₀ cycloalkyl)O— in each instance.

47. Compound according to any one of items 1 to 39, wherein R⁶ is (C₃-C₅ cycloalkyl)O— in each instance.

48. Compound according to any one of items 1 to 39, wherein R⁶ is (C₅ cycloalkyl)O— in each instance.

49. A compound according to any one of items 1 to 48, wherein said compound is selected from the group consisting of:
Diethyl ((E)-3-(4-((6-(4-((6-amino-2-butoxy-8-hydroxy-9H-purin-9-yl)methyl)benzamido)hexanoyl)-oxy)-3-methoxyphenyl)acryloyl)glycyl-L-valyl-D-glutamate,
Ethyl N5-(2-(2-(4-((6-amino-2-butoxy-8-hydroxy-9H-purin-9-yl)methyl)benzamido)ethoxy)ethyl)-N2-((E)-3-(4-hydroxy-3-methoxyphenyl)acryloyl)glycyl-L- valyl-D-glutaminate,
Ethyl (R)-1-(4-((6-amino-2-butoxy-8-hydroxy-9H-purin-9-yl)methyl)phenyl)-15-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate,
Ethyl (R)-1-(4-((6-amino-2-butoxy-8-hydroxy-9H-purin-9-yl)methyl)phenyl)-15-((S)-2-(2-((E)-3-(4-isopropylphenyl)acrylamido)acetamido)-3- methylbutanamido)-1,12-dioxo-5,8-dioxa-2,11-diazahexadecan-16-oate,
Ethyl (R)-1-(4-((6-amino-2-butoxy-8-hydroxy-9H-purin-9-yl)methyl)phenyl)-15-((S)-2-(2-((E)-3-(3,4-difluorophenyl)acrylamido)acetamido)-3- methylbutanamido)-1,12-dioxo-5,8-dioxa-2,11-diazahexadecan-16-oate,
Ethyl (15R)-1-(4-((6-amino-2-butoxy-8-hydroxy-9H-purin-9-yl)methyl)phenyl)-15-((2S)-2-(2-(2-(3,4-difluorophenyl)cyclopropane-1- carboxamido)acetamido)-3-methylbutanamido)-1,12-dioxo-5,8-dioxa-2,11-diazahexadecan-16-oate,
Ethyl (R)-1-(4-((6-amino-2-butoxy-8-hydroxy-9H-purin-9-yl)methyl)phenyl)-13-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate,
Ethyl (R)-1-(4-((6-amino-2-butoxy-8-hydroxy-9H-purin-9-yl)methyl)phenyl)-13-((S)-2-(2-cinnamamidoacetamido)-3-methylbutanamido)-1,12-dioxo-5,8- dioxa-2,11-diazahexadecan-16-oate,
Ethyl (13R)-1-(4-((6-amino-2-butoxy-8-hydroxy-9H-purin-9-yl)methyl)phenyl)-13-((2S)-2-(2-(2-(3,4-difluorophenyl)cyclopropane-1- carboxamido)acetamido)-3-methylbutanamido)-1,12-dioxo-5,8-dioxa-2,11-diazahexadecan-16-oate,
Diethyl ((E)-3-(4-(1-(4-((6-amino-2-butoxy-8-hydroxy-9H-purin-9-yl)methyl)phenyl)-1,17-dioxo-6,9,12-trioxa-2,16-diazaicosan-20- amido)phenyl)acryloyl)glycyl-L-valyl-D-glutamate,
Dicyclopentyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3-methoxyphenyl)acryloyl)glycyl-L-valyl-D-glutamate,
Diethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3- methoxyphenyl)acryloyl)glycyl-L-alanyl-D-glutamate,
Diethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3- methoxyphenyl)acryloyl)glycyl-L-phenylalanyl-D-glutamate, and
1-benzyl 5-ethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3-methoxyphenyl)acryloyl)glycyl-L-valyl-D-glutamate.

A compound according to any one of items 1 to 48, wherein said compound is selected from the group consisting of:
Diethyl ((E)-3-(4-((6-(4-((6-amino-2-butoxy-8-hydroxy-9H-purin-9-yl)methyl)benzamido)hexanoyl)-oxy)-3-methoxyphenyl)acryloyl)glycyl-L-valyl-D-glutamate,
Ethyl N5-(2-(2-(4-((6-amino-2-butoxy-8-hydroxy-9H-purin-9-yl)methyl)benzamido)ethoxy)ethyl)-N2-((E)-3-(4-hydroxy-3-methoxyphenyl)acryloyl)glycyl-L- valyl-D-glutaminate, and
Ethyl (R)-1-(4-((6-amino-2-butoxy-8-hydroxy-9H-purin-9-yl)methyl)phenyl)-15-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate.

51. A process for preparing a compound of Formula I as defined in any one of items 1 to 50 (with the variable groups being as defined in any one of items 1 to 50) which comprises reacting a compound of Formula V:

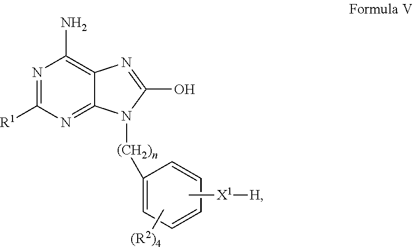

with a compound of Formula VI:

H—L—R³ Formula VI,

or a compound of Formula VII:

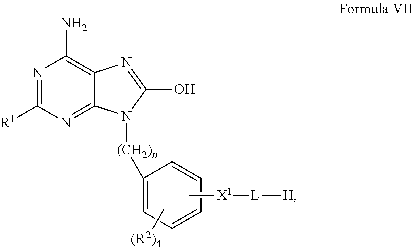

with a compound of Formula VIII:

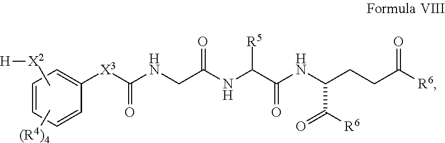

### Formula IX:

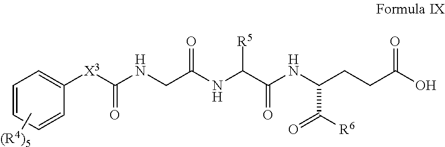

### or Formula X:

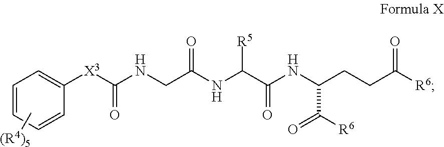

and optionally thereafter carrying out one or more of the following procedures:
removing any protecting groups,
forming a pharmaceutically acceptable salt,
converting a compound of Formula I into another compound of Formula I.

52. Compound of any one of item 1 to 50 for use in medicine.

53. Compound of any one of items 1 to 50 for use in treatment of a condition in which agonism of TLR7 and NOD2 receptors is beneficial.

54. Compound for use of item 53, wherein the condition is selected from the group consisting of viral infections, bacterial infections, fungal infections, protozoal infections, tumors, cancers and immunological diseases

55. A pharmaceutical composition comprising a compound according to any one of items 1 to 50 and one or more pharmaceutically acceptable excipients or carriers.

56. A vaccine comprising a compound according to any one of items 1 to 50.

### BRIEF DESCRIPTION OF FIGURES

FIG. 1 demonstrates the ability of the compounds of the invention (SG48, SG144, RH32) to activate the cytotoxic activity of PBMCs against the MEC-1 cancer cell line. Concentration of tested compounds was 1 μM. The results are expressed as a ratio against the control (medium). Intermediary compounds SG8, SG43 and their unconjugated mixture were employed to compare the compounds of the invention to a NOD2 agonist, TLR7 agonist and their unconjugated mixture, respectively.

FIG. 2 demonstrates the ability of the compounds of the invention (SG48, SG144, RH32) to activate the cytotoxic activity of PBMCs against the K562 cancer cell line. Concentration of tested compounds was 1 μM. The results are expressed as a ratio against the control (medium). Intermediary compounds SG8, SG43 and their unconjugated mixture were employed to compare the compounds of the invention to a NOD2 agonist, TLR7 agonist and their unconjugated mixture, respectively.

FIG. 3 demonstrates the ability of the compounds of the invention (SG48, SG144, RH32) to promote BMDC antigen presentation to CD4 positive T-lymphocytes. Concentration of tested compounds was 100 nM. The results are expressed as a ratio of CD25 positive T-lymphocytes with low CFSE fluorescence. Medium was used as the negative control. LPS (1 μg/mL) was used as the positive control. Intermediary compounds SG8, SG43 and their unconjugated mixture were employed to compare the compounds of the invention to a NOD2 agonist, TLR7 agonist and their unconjugated mixture, respectively.

FIG. 4 demonstrates the ability of the compounds of the invention (SG48, SG144, RH32) to promote BMDC antigen presentation to CD8 positive T-lymphocytes. Concentration of tested compounds was 100 nM. The results are expressed as a ratio of CD25 positive T-lymphocytes with low CFSE fluorescence. Medium was used as the negative control. LPS (1 μg/mL) was used as the positive control. Intermediary compounds SG8, SG43 and their unconjugated mixture were employed to compare the compounds of the invention to a NOD2 agonist, TLR7 agonist and their unconjugated mixture, respectively.

FIG. 5 demonstrates the dose-dependent ability of the compounds of the invention (SG48, SG144) to promote BM DC antigen presentation to CD4 positive T-lymphocytes. The results are expressed as a ratio of CD25 positive T-lymphocytes with low CFSE fluorescence.

FIG. 6 demonstrates the dose-dependent ability of the compounds of the invention (SG48, SG144) to promote BM DC antigen presentation to CD8 positive T-lymphocytes. The results are expressed as a ratio of CD25 positive T-lymphocytes with low CFSE fluorescence.

FIGS. 7A-7G demonstrate the CD4+ and CD8+ T-lymphocyte secretion of IL-2 (FIG. 7A), IL-4 (FIG. 7B) , IL-6 (FIG. 7C) , IL-10 (FIG. 7D) , IL-17A (FIG. 7E) , IFN-γ (FIG. 7F) and TNF (FIG. 7G) in response to ovalbumin presentation by BM DCs pretreated with compound of the invention (SG144). Medium was used as a negative control. LPS (1 μg/mL) was used as the positive control. Intermediary compounds SG8, SG43 and their unconjugated mixture were employed to compare the compounds of the invention to a NOD2 agonist, TLR7 agonist and their unconjugated mixture, respectively.

### DETAILED DESCRIPTION OF THE INVENTION

### Definitions

Throughout the present specification and the accompanying claims, the word “comprise” and variations such as “comprises” and “comprising” are to be interpreted inclusively. That is, these words are intended to convey the possible inclusion of other elements or integers not specifically recited, where the context allows.

“Cᵢ-Cⱼ alkyl” means linear or branched alkyl group comprising of i to j carbon atoms. Non-limiting examples of alkyl group include methyl, ethyl, n-propyl, isopropyl, n-butyl, isobutyl, pentyl and hexyl.

“Cᵢ-Cⱼ alkoxy” means a Cᵢ-Cⱼ alkyl group as defined above linked to an oxygen atom. Non-limiting examples of alkoxy groups include methoxy, ethoxy, propoxy, isopropoxy, n-butoxy, tert-butoxy, pentoxy and hexoxy.

“Cᵢ-Cⱼ alkenyl” means a linear or branched hydrocarbon group comprising of i to j carbon atoms containing at least one carbon-carbon double bond. Asymmetric structures are intended to include all positional and geometrical isomers. Non-limiting examples of alkenyl group include vinyl, propenyl, allyl, but-2-enyl and but-3-enyl.

“Cᵢ-Cⱼ alkynyl” means a linear or branched hydrocarbon group comprising of i to j carbon atoms containing at least one carbon-carbon triple bond. Non-limiting examples of alkynyl group include ethynyl, prop-2-ynyl and but-2-ynyl.

“Cᵢ-Cⱼ aryl” means a mono-, or bicyclic aromatic carbon-based radical comprising of i to j carbon atoms. Non-limiting examples or aryl group include phenyl and naphthyl.

“Cᵢ-Cⱼ cycloalkyl” means a mono-, bi-, or tricyclic, non-aromatic carbon-based radical comprising of i to j carbon atoms that is fully saturated or partially unsaturated. Non-limiting examples of cycloalkyl groups include cyclopropyl, cyclobutyl, cyclopentyl and cyclohexyl.

“Cᵢ-Cⱼ heterocycle” means a saturated, partially saturated or unsaturated cycle consisting of one or several rings, comprising of i to j atoms of which one or more ring atoms are selected from nitrogen, oxygen or sulphur wherein a ring sulphur atom may be optionally oxidised to from the S-oxide, a ring nitrogen may be optionally oxidised to from the N-oxide, a ring nitrogen may be optionally quaternized and a —CH₂— group may be optionally replaced by a —C(═O)—. Non-limiting examples of heterocycles include pyrrolidine, pyrrole, furan, thiophene, piperidine, pyridine, pyridine-N-oxide, pyran, morpholine, piperazine, pyrimidine, pyrazole, imidazole, oxazole, isoxazole, oxodiazole, benzimidazole, benzoxazole, indole, isoindole, indoline, benzotriazole and quinoline.

“Cᵢ-Cⱼ heterocyclyl” means a saturated, partially saturated or unsaturated cyclic radical consisting of one or several rings, comprising of i to j atoms of which one or more ring atoms are selected from nitrogen, oxygen or sulphur wherein a ring sulphur atom may be optionally oxidised to from the S-oxide, a ring nitrogen may be optionally oxidised to from the N-oxide, a ring nitrogen may be optionally quaternized and a —CH₂— group may be optionally replaced by a —C(═O)—. Non-limiting examples of heterocyclyl groups include pyrrolidinyl, pyrrolyl, furanyl, thiophenyl, piperidinyl, pyridinyl, pyranyl, morpholinyl, piperazinyl, pyrimidiyl, pyrazolyl, imidazolyl, oxazolyl, isoxazolyl, oxodiazolyl, benzimidazolyl, benzoxazolyl, indolyl, isoindolyl, indolinyl, benzotriazolyl and quinolinyl.

“Halogen” or “halo” means fluorine, chlorine, bromine or iodine.

The term “carboxy” or “carboxyl” as used herein, refers to the —COOH group.

“Specific side chain of an amino acid” means the R group of an amino acid with a generic formula H₂NCHRCOOH. This includes but is not limited to the L and D isomers of natural amino acids arginine, histidine, lysine, aspartic acid, glutamic acid, serine, threonine, asparagine, glutamine, cysteine, glycine, proline, alanine, valine, isoleucine, leucine, methionine, phenylalanine, tyrosine or tryptophan as well as unnatural amino acids such as homoserine, ornithine, citrulline, phosphoserine, phosphothreonine, phosphotyrosine, isovaline, isoserine, allothreonine or hydroxyproline.

The term “peptide” as used herein, refers to an amino acid polymer wherein each amino acid is linked to its neighbor by an amide bond —C(═O)NH—, also called peptide bond.

The term “pharmaceutically acceptable” as used herein, refers to those compounds, materials, compositions and/or dosage forms with are, within the scope of sound medical judgement, suitable for use in contact with the tissues of human beings and animals without excessive toxicity, irritation, allergic response or other problem or complication commensurate with a reasonable benefit/risk ratio.

“Pharmaceutically acceptable salts” means a salt of a disclosed compound that does not abrogate the biological effectiveness and retains properties of free bases or free acids, which are not biologically or otherwise undesirable. Where a compound has one or more basic groups, acid addition salts can be obtained by contacting the neutral form of such compounds with a sufficient amount of the desired acid, either neat or in a suitable inert solvent. The salt can be formed with inorganic acids such as hydrochloric acid, hydrobromic acid, sulfuric acid, nitric acid, phosphoric acid, and the like or organic acid such as acetic acid, tartaric acid, propionic acid, glycolic acid, pyruvic acid, oxalic acid, benzoic acid, cinnamic acid, methanesulfonic acid, lactic acid, maleic acid, fumaric acid, succinic acid, and the like. Where a compound has one or more acidic groups, the salt can be formed with the addition of an organic or an inorganic base. Salts derived from inorganic bases include but are not limited to sodium, potassium, lithium, ammonium, calcium, magnesium and zinc salts. Salts derived from organic bases include but are not limited to salts of primary, secondary and tertiary amines, substituted amines, cyclic amines, naturally-occurring amines and the like, such as isopropylamine, trimethylamine, diethylamine, triethylamine, ethanolamine, lysine, arginine, piperidine, piperazine, choline, betaine, caffeine, choline, and the like. Certain specific compounds of the present invention contain both basic and acidic functionalities that allow the compounds to be converted into either base or acid addition salts. A preferred pharmaceutically acceptable salt is the sodium salt.

Other salts that are not deemed pharmaceutically acceptable may be useful in the preparation of compound of Formula I and are included within the scope of this invention, such as those formed with ammonia and trifluoroacetic acid.

“Pharmaceutically acceptable esters” means that compounds of Formula I may be derivatised at carboxylic or hydroxy groups to provide derivatives which are capable of conversion back to the parent compounds in vivo. Examples of such compounds include physiologically acceptable and metabolically labile ester derivatives, such as methoxymethyl esters, methylthiomethyl ester and pivaloyloxymethyl esters. Additionally, any physiologically acceptable equivalents of the compounds of Formula I, similar to the metabolically labile esters, which are capable of producing the parent compounds of Formula I in vivo, are within the scope of this invention.

The term “optionally substituted” as used herein, refers to a group that may or may not be further substituted with one or more groups (preferably 1, 2, 3 or 4 groups, more preferably 1 or 2 groups). Permissible substituents include but are not limited to OR, SR, NR₂, CN, NO₂, halogen, oxo, carboxyl, CF₃, CH₂CF₃, CHF₂, OCF₃, OCHF₂, ═NR, ═N, ═NOR, ═N—CN, —C(═O)NR₂, —NRC(═O)R, —C(═O)R, —OC(═O)R, —C(═O)OR, —NRC(═O)OR, —SO₂R, —SO₂NR₂, —NRSO₂R, C₁-C₆ alkyl, C₂-C₆ alkenyl, C₂-C₆ alkynyl, C₁-C₆ alkoxy, C₃-C₁₀ cycloalkyl, C₆-C₁₀ aryl and C₅-C₉ heterocyclyl wherein each R is H, C₁-C₆ alkyl, C₂-C₆ alkenyl, C₂-C₆ alkynyl, C₃-C₁₀ cycloalkyl, C₆-C₁₀ aryl or C₅-C₉ heterocyclyl and wherein each alkyl, alkenyl, alkynyl, cycloalkyl, aryl and heterocyclyl and groups containing them may be further optionally substituted, e.g., with one or more groups (preferably 1, 2, 3 or 4 groups, more preferably 1 or 2 groups) selected from OR, SR, NR₂, CN, NO₂, halogen, oxo, carboxyl, CF₃, CH₂CF₃, CHF₂, OCF₃, OCHF₂, ═NR, ═N, ═NOR, ═N—CN, —C(═O)NR₂, —NRC(═O)R, —C(═O)R, —OC(═O)R, —C(═O)OR, —NRC(═O)OR, —SO₂R, —SO₂NR², and —NRSO₂R.

The term “administering” as used herein, refers to parenteral, intravenous, intraperitoneal, intramuscular, intertumoral, intralesional, intranasal, subcutaneous or oral administration, administration as a suppository, topical contact, intrathecal administration, or the implantation of a slow-release device, such as a mini-osmotic pump, to the subject.

The term “subject” as used herein, refers to an animal or human body.

The term “agonist” as used herein, refers to the native ligand of a receptor, to analogues thereof or other ligand that similarly “activate” the receptor, and/or to a positive modulator of the receptor.

A NOD2 agonist is any compound that functions to activate NOD2 receptor

A TLR7 agonist is any compound that functions to activate TLR7 receptor.

The term “therapeutically effective amount” as used herein, refers to the quantity of a compound of Formula I or a pharmaceutically acceptable salt, racemate, diastereomer, enantiomer, ester or prodrug thereof, which will elicit the desired biological response in an animal or human body.

The term “treatment” or “treating” as used herein, includes (i) preventing a pathological condition from occurring; (ii) inhibiting the pathological condition or arresting its development; (iii) relieving the pathological condition and/or diminishing symptoms associated with the pathological condition.

“Pharmaceutical composition” means one or more active ingredients, and one or more inert ingredients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable excipient (pharmaceutically acceptable carrier).

“Excipient” refers to compounds administered together with the therapeutic agent, for example, buffering agents, isotonicity modifiers, preservative, stabilizers, anti-adsorption agents, or other auxiliary agents. However, in some cases, one excipient may have dual or triple functions.

It will be appreciated by those skilled in the art that certain compounds described herein contain one or more chiral centres. Accordingly, these compounds may exist in, and be isolated in, optically active and racemic forms. Some compounds may exhibit polymorphism. Polymorphs as referred herein includes both crystalline and amorphous forms. It is to be understood that this invention encompasses any racemic, optically active, polymorphic or stereoisomeric form, or mixtures thereof, of a compound of the invention, which possesses the useful properties described herein, it being known in the art how to prepare optically-active forms (for example, by resolution of the racemic form by recrystallization techniques, by synthesis from optically-active starting materials, by stereoselective synthesis, by enzymatic resolution, by biotransformation, or by chromatographic separation using a chiral stationary phase).

In addition to salt forms, the invention provides compounds which are in a prodrug form. Prodrugs of the compounds described herein are those compounds that readily undergo chemical changes under physiological conditions to provide the compounds of the present invention. Prodrugs include, for example, compounds of this invention wherein hydroxy, amine or carboxy groups are bonded to any group that, when administered to a subject, cleaves to form the hydroxy, amine or carboxy groups. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. A prodrug may improve the physical properties of the parent drug and/or it may also improve overall drug efficacy, for example through the reduction of toxicity and unwanted effects of a drug by controlling its absorption, blood levels, metabolic distribution and cellular uptake.

Within the present invention it is to be understood that a compound of Formula I or a salt thereof may exhibit the phenomenon of tautomerism and that the formulae drawings within this specification can represent only one of the possible tautomeric forms. It is to be understood that the invention encompasses any tautomeric form which possesses the useful properties described herein and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

In particular, a compound of Formula Ia is the tautomer of a compound of Formula Ib.

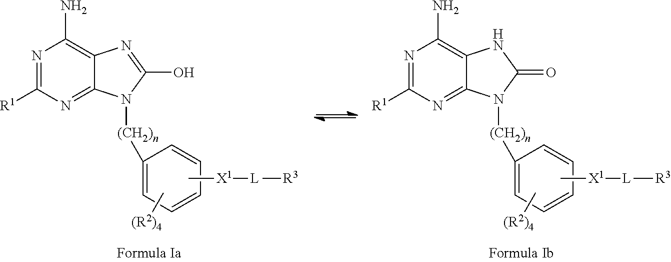

Generally, tautomeric structures have been represented herein in the enol form, as a matter of consistency and convenience.

It is also to be understood that certain compounds of Formula I and salts thereof can exist in solvated as well as unsolvated forms such as, for example, hydrated forms. It is to be understood that the invention encompasses all such solvated form which possess the useful properties described herein.

In the formulae drawings within this specification a bond traversing an aromatic ring between two carbon atoms means that the attached group may be located at any of the positions on the aromatic ring, made available by removal of the hydrogen atom that is implicitly there. By way of illustration, the formula

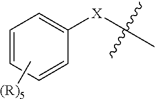

represents

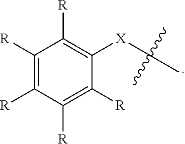

In another illustrations, the formula

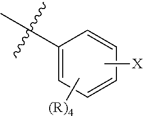

represents

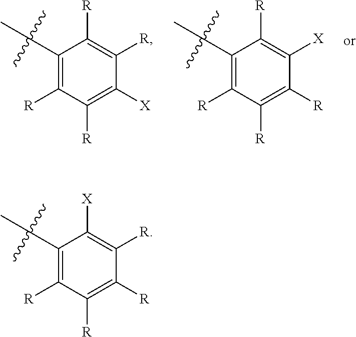

### Conjugates of NOD2 and TLR7 Agonists

The first aspect of the present invention are conjugated compounds of formula I:

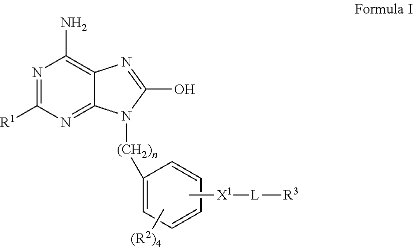

or a pharmaceutically acceptable salt, racemate, diastereomer, enantiomer, ester or prodrug thereof, wherein:
R¹ is H, halogen, OH, SH, CF₃, C₁-C₆ alkyl, C₃-C₁₀ cycloalkyl, C₆-C₁₀ aryl, C₅-C₉ heterocyclyl, C₁-C₆ alkoxy, C₁-C₆ alkoxy C₁-C₆ alkyl, C₁-C₆ alkoxy-C₁-C₆ alkoxy, C₁-C₆ alkoxy-(C₁-C₆ alkyl)S—, (C₁-C₆ alkyl)SO₂NH—, (C₁-C₆ alkyl)C(═O)O—, (C₁-C₆ alkyl)OC(═O)—, (C₁-C₆ alkyl)C(═O)—, (C₁-C₆ alkyl)C(═O)NH—, R^{a}R^{b}N— or R^{a}R^{b}N(C═O)—, wherein alkyl, alkoxy, cycloalkyl, aryl and heterocyclyl may be optionally substituted;
R² is independently for each occurrence selected from H, halogen, OH, CHF₂, CF₃, CH₂CF₂, carboxy, CN, NO₂, C₁-C₆ alkyl, C₃-C₁₀ cycloalkyl, C₁-C₆ alkoxy, (C₁-C₆ alkyl)C(═O)—, (C₆-C₁₀ aryl)C(═O)—, (C₁-C₆ alkyl)S—, (C₁-C₆ alkyl)C(═O)O—, (C₁-C₆ alkyl)OC(═O)—, (C₁-C₆ alkyl)C(═O)NH—, (C₁-C₆ alkyl)SO₂NH—, R^{a}R^{b}N— and R^{a}R^{b}N(C═O)—, wherein alkyl, alkoxy, aryl and cycloalkyl may be optionally substituted;
R^{a} and R^{b} are independently from each other selected from H, C₁-C₆ alkyl, C₃-C₁₀ cycloalkyl, C₆-C₁₀ aryl and C₅-C₉ heterocyclyl or R^{a} and R^{b} may together with the nitrogen atom form a C₅-C₆ heterocycle, wherein alkyl, cycloalkyl, aryl and heterocyclyl may be optionally substituted; preferably R^{a} and R^{b} are independently from each other selected from H, C₃-C₁₀ cycloalkyl, C₆-C₁₀ aryl and C₅-C₉ heterocyclyl or R^{a} and R^{b} may together with the nitrogen atom form a C₅-C₆ heterocycle, wherein alkyl, cycloalkyl, aryl and heterocyclyl may be optionally substituted;
X¹ is a single bond, —O—, —S—, —NR_{c}-, —C(═O)— or —SO₂—;
R^{c} is H, C₁-C₆ alkyl or C₃-C₁₀ cycloalkyl;
L is a linking group;
n is 0, 1, 2, 3 or 4; and
R³ is a NOD2 agonist selected from the group consisting of:

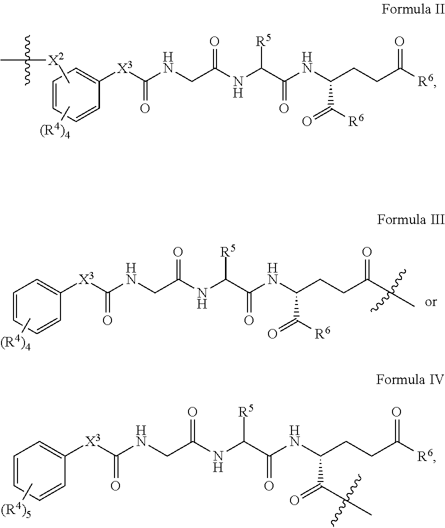

wherein:
X² is a single bond, —O—, —S—, —NR_{d}-, —C(═O)— or —SO₂—;
R^{d} is H, C₁-C₆ alkyl or C₃-C₁₀ cycloalkyl;
X³ is —(CH₂)₂—, —(CH═CH)— or cyclopropylene;
R⁴ is independently for each occurrence selected from H, halogen, OH, CHF₂, CF₃, CH₂CF₂, carboxy, CN, NO₂, C₁-C₆ alkyl, C₁-C₆ alkoxy, C₃-C₁₀ cycloalkyl, (C₁-C₆ alkyl)C(═O)—, (C₁-C₆ aryl)C(═O)—, (C₁-C₆ alkyl)S—, (C₁-C₆ alkyl)C(═O)O—, (C₁-C₆ alkyl)OC(═O)—, (C₁-C₆ alkyl)C(═O)NH—, (C₁-C₆ alkyl)SO₂NH—, R^{e}R^{f}N— and R^{e}R^{f}N(C═O)—, wherein alkyl, alkoxy, aryl and cycloalkyl may be optionally substituted;
R^{e} and R^{f} are independently from each other selected from H, C₁-C₆ alkyl, C₃-C₁₀ cycloalkyl, C₆-C₁₀ aryl and C₅-C₉ heterocyclyl or R^{e} and R^{f} may together with the nitrogen atom form a C₅-C₆ heterocycle;
R⁵ is C₁-C₆ alkyl or a specific side chain of an amino acid;
R⁶ is independently for each occurrence selected from OH, C₁-C₁₈ alkoxy, (C₂-C₁₈ alkenyl)O—, (C₃-C₁₀ cycloalkyl)O—, (C₆-C₁₀ aryl)O—, (C₅-C₉ heterocyclyl)O— and R^{g}R^{h}N—, wherein alkoxy, alkenyl, cycloalkyl, aryl or heterocyclyl may be optionally substituted; and
R^{g} and R^{h} are independently from each other selected from H, C₁-C₁₈ alkyl, C₂-C₁₈ alkenyl, C₃-C₁₀ cycloalkyl, C₆-C₁₀ aryl and C₅-C₉ heterocyclyl or R^{g} and R^{h} may together with the nitrogen atom form a C₅-C₆ heterocycle wherein alkyl, alkenyl, cycloalkyl, aryl and heterocyclyl may be optionally substituted.

In one embodiment, the present invention provides a subset of compounds of Formula I, of Formula Ic:

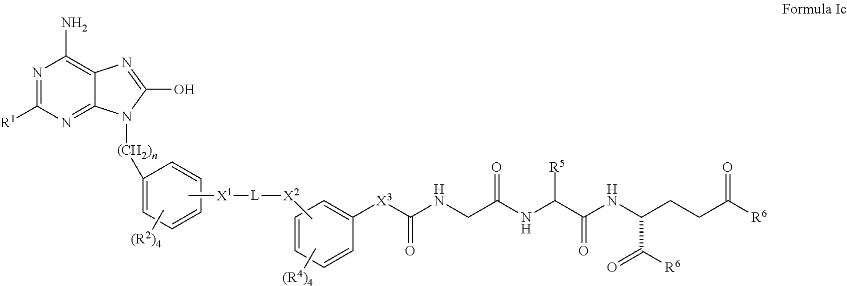

or a pharmaceutically acceptable salt, racemate, diastereomer, enantiomer, ester or prodrug thereof, wherein R¹, R², R⁴, R⁵, R⁶, X¹, X², X³, L and n are as defined previously.

In another embodiment, the present invention provides a subset of compound of Formula I, of Formula Id:

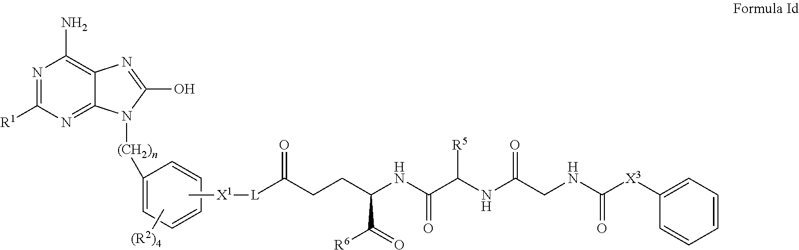

or a pharmaceutically acceptable salt, racemate, diastereomer, enantiomer, ester or prodrug thereof, wherein R¹, R², R⁴, R⁵, R⁶, X¹, X³, L and n are as defined previously.

In another embodiment, the present invention provides a subset of compound of Formula I, of Formula Ie:

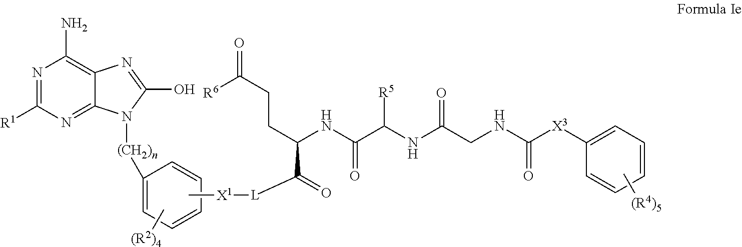

or a pharmaceutically acceptable salt, racemate, diastereomer, enantiomer, ester or prodrug thereof, wherein R¹, R², R³, R⁴, R⁵, R⁶, X¹, X³, L and n are as defined previously.

According to certain embodiments, n is 1 or 2.

According to certain embodiments, n is 1.

According to certain embodiments, R¹ is hydrogen, C₁-C₆ alkoxy, C₁-C₆ alkoxy-C₁-C₆ alkoxy, (C₁-C₆ alkyl)NH, C₁-C₆ alkoxy-(C₁-C₆ alkyl)NH, (C₁-C₆ alkyl)S— or CF₃.

According to certain embodiments, R¹ is hydrogen, C₁-C₆ alkoxy, C₁-C₆ alkoxy-C₁-C₆ alkoxy, (C₁-C₆ alkyl)S— or CF₃.

According to certain embodiments, R¹ is C₁-C₆ alkoxy or C₁-C₆ alkoxy-C₁-C₆ alkoxy.

According to certain embodiments, R¹ is C₁-C₆ alkoxy.

According to certain embodiments, R¹ is n-butoxy.

According to certain embodiments, R¹ is C₁-C₆ alkoxy-C₁-C₆ alkoxy.

According to certain embodiments, R¹ is CH₂O(CH₂)₂O—.

According to certain embodiments, R² is independently for each occurrence selected from H, halogen or C₁-C₆ alkyl.

According to certain embodiments, R² is H in each instance.

According to certain embodiments, X¹ is —O—, —NH— or —C(═O)—.

According to certain embodiments, X¹ is —C(═O)—.

According to certain embodiments, X¹ is in para position relative to the (CH₂)ₙ group.

According to certain embodiments, X² is —O—, —NH— or —C(═O)—.

According to certain embodiments, X² is —O—.

According to certain embodiments, X² is in para position relative to the X³ group.

According to certain embodiments, X³ is —CH═CH— or cyclopropylene.

According to certain embodiments, X³ is —CH═CH—.

According to certain embodiments, X³ is cyclopropylene.

According to certain embodiments, R⁴ is independently for each instance selected from hydrogen, halogen, OH, C₁-C₆ alkyl and C₁-C₆ alkoxy.

According to certain embodiments, R⁴ is independently for each instance selected from hydrogen, OH, and C₁-C₆ alkoxy.

According to certain embodiments, R⁴ is C₁-C₆ alkoxy in meta position relative to the X³ group, OH in para position relative to the X³ group and H in other instances.

According to certain embodiments, R⁴ is methoxy in meta position relative to the X³ group, OH in para position relative to the X³ group and H in other instances.

According to certain embodiments, R⁴ is isopropyl in para position relative to the X³ group and H in other instances.

According to certain embodiments, R⁴ is H in each instance.

According to certain embodiments, R⁴ is fluoro in meta position relative to the X³ group, fluoro in para position relative to the X³ group and H in other instances.

According to certain embodiments, R⁵ is C₁-C₆ alkyl or a specific side chain of a natural amino acid.

According to certain embodiments, R⁵ is C₁-C₆ alkyl or the specific side chain of valine, alanine, phenylalanine, leucine or isoleucine.

According to certain embodiments, R⁵ is C₁-C₆ alkyl or the specific side chain of valine, alanine, leucine or isoleucine.

According to certain embodiments, R⁵ is C₁-C₆ alkyl.

According to certain embodiments, R⁵ is a specific side chain of a natural amino acid.

According to certain embodiments, R⁵ is the specific side chain of valine, alanine, phenylalanine, leucine or isoleucine.

According to certain embodiments, R⁵ is the specific side chain of valine, alanine, leucine or isoleucine.

According to certain embodiments, R⁵ is the specific side chain of valine.

According to certain embodiments, R⁵ is the specific side chain of phenylalanine.

According to certain embodiments, R⁵ is the specific side chain of alanine.

According to certain embodiments, R⁶ is independently for each instance selected from OH, NH₂, (C₂-C₁₈ alkenyl)O—, (C₃-C₁₀ cycloalkyl)O— and C₁-C₁₈ alkoxy.

According to certain embodiments, R⁶ is independently for each instance selected from OH, NH₂, (C₂-C₁₀ alkenyl)O—, (C₃-C₁₀ cycloalkyl)O— and C₁-C₁₀ alkoxy.

According to certain embodiments, R⁶ is independently for each instance selected from OH, NH₂, (C₂-C₆ alkenyl)O—, (C₃-C₁₀ cycloalkyl)O— and C₁-C₆ alkoxy.

According to certain embodiments, R⁶ is independently for each instance selected from OH, (C₃-C₁₀ cycloalkyl)O— and C₁-C₁₈ alkoxy.

According to certain embodiments, R⁶ is independently for each instance selected from OH, (C₃-C₅ cycloalkyl)O— and C₁-C₁₀ alkoxy.

According to certain embodiments, R⁶ is independently for each instance selected from OH and C₁-C₆ alkoxy.

According to certain embodiments, R⁶ is independently for each instance selected from (C₃-C₅ cycloalkyl)O— and C₁-C₆ alkoxy.

According to certain embodiments, R⁶ is independently for each instance selected from OH and (C₃-C₅ cycloalkyl)O—.

According to certain embodiments, R⁶ is independently for each instance selected from R⁶ is OH and C₁-C₁₀ alkoxy.

According to certain embodiments, R⁶ is independently for each instance selected from R⁶ is OH and C₁-C₆ alkoxy.

According to certain embodiments, R⁶ is OH in each instance.

According to certain embodiments, R⁶ is C₁-C₆ alkoxy in each instance.

According to certain embodiments, R⁶ is ethoxy in each instance.

According to certain embodiments, R⁶ is (C₃-C₁₀ cycloalkyl)O— in each instance.

According to certain embodiments, R⁶ is (C₃-C₅ cycloalkyl)O— in each instance.

According to certain embodiments, R⁶ is (C₅ cycloalkyl)O— in each instance.

In some preferred embodiments of Formula I:
R¹ is C₁-C₆ alkoxy or C₁-C₆ alkoxy-C₁-C₆ alkoxy, preferably n-butoxy or CH₂O(CH₂)₂O—;
R² is H in each instance;
n is 1;
X¹ is —C(═O)—; preferably is —C(═O)— in para position relative to the (CH₂)ₙ group;
L is a linking group;
R³ is as defined herein.

In some preferred embodiments, R³ is a NOD2 agonist of Formula II, wherein:
X² is —O—, —NH— or —C(═O)— in para position relative to the X³ group; preferably is —O— in para position relative to the X³ group;
R⁴ is methoxy in meta position relative to the X³ group and H in other instances;
R⁵ is the side chain of L-valine, L-alanine or L-phenylalanine, preferably L-valine;
R⁶ is independently for each occurrence selected from OH, NH2, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH, C₁-C₁₈ alkoxy or (C₃-C₁₀ cycloalkyl)O—; more preferably R⁶ is OH, C₁-C₆ alkoxy or (C₃-C₅ cycloalkyl)O—; even more preferably R⁶ is C₁-C₆ alkoxy or (C₅ cycloalkyl)O—.

In other preferred embodiments, R³ is a NOD2 agonist of Formula III or wherein:
R⁴ is methoxy in meta position relative to the X³ group, OH in para position relative to the X³ group and H in other instances;
R⁵ is the side chain of L-valine, L-alanine or L-phenylalanine, preferably L-valine;
R⁶ is OH, NH₂, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH or C₁-C₁₈ alkoxy; more preferably R⁶ is OH or C₁-C₆ alkoxy; even more preferably R⁶ is C₁-C₆ alkoxy.

In other preferred embodiments, R³ is a NOD2 agonist of Formula III or wherein:
R⁴ is fluoro in meta position relative to the X³ group, fluoro in para position relative to the X³ group and H in other instances;
R⁵ is the side chain of L-valine, L-alanine or L-phenylalanine, preferably L-valine;
R⁶ is OH, NH₂, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH or C₁-C₁₈ alkoxy; more preferably R⁶ is OH or C₁-C₆ alkoxy; even more preferably R⁶ is C₁-C₆ alkoxy.

In other preferred embodiments, R³ is a NOD2 agonist of Formula III or wherein:
R⁴ is isopropyl in para position relative to the X³ group and H in other instances;
R⁵ is the side chain of L-valine, L-alanine or L-phenylalanine, preferably L-valine;
R⁶ is OH, NH₂, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH or C₁-C₁₈ alkoxy; more preferably R⁶ is OH or C₁-C₆ alkoxy; even more preferably R⁶ is C₁-C₆ alkoxy.

In other preferred embodiments, R³ is a NOD2 agonist of Formula III orwherein:
R⁴ is H in each instance;
R⁵ is the side chain of L-valine, L-alanine or L-phenylalanine, preferably L-valine;
R⁶ is OH, NH₂, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH or C₁-C₁₈ alkoxy; more preferably R⁶ is OH or C₁-C₆ alkoxy; even more preferably R⁶ is C₁-C₆ alkoxy.

In other preferred embodiments, R³ is a NOD2 agonist of Formula IV, wherein:
R⁴ is methoxy in meta position relative to the X³ group, OH in para position relative to the X³ group and H in other instances;
R⁵ is the side chain of L-valine, L-alanine or L-phenylalanine, preferably L-valine;
R⁶ is OH, NH₂, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH or C₁-C₁₈ alkoxy; more preferably R⁶ is OH or C₁-C₆ alkoxy; even more preferably R⁶ is C₁-C₆ alkoxy.

In other preferred embodiments, R³ is a NOD2 agonist of Formula IV, wherein:
R⁴ is fluoro in meta position relative to the X³ group, fluoro in para position relative to the X³ group and H in other instances;
R⁵ is the side chain of L-valine, L-alanine or L-phenylalanine, preferably L-valine;
R⁶ is OH, NH₂, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH or C₁-C₁₈ alkoxy; more preferably R⁶ is OH or C₁-C₆ alkoxy; even more preferably R⁶ is C₁-C₆ alkoxy.

In other preferred embodiments, R³ is a NOD2 agonist of Formula IV, wherein:
R⁴ is isopropyl in para position relative to the X³ group and H in other instances;
R⁵ is the side chain of L-valine, L-alanine or L-phenylalanine, preferably L-valine;
R⁶ is OH, NH₂, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH or C₁-C₁₈ alkoxy; more preferably R⁶ is OH or C₁-C₆ alkoxy; even more preferably R⁶ is C₁-C₆ alkoxy.

In other preferred embodiments, R³ is a NOD2 agonist of Formula IV, wherein:
R⁴ is H in each instance;
R⁵ is the side chain of L-valine, L-alanine or L-phenylalanine, preferably L-valine;
R⁶ is OH, NH₂, C₁-C₁₈ alkoxy, (C₂-C₁₈ alkenyl)O—, (C₃-C₁₀ cycloalkyl)O—, (C₆-C₁₀ aryl)O— and (C₅-C₉ heterocyclyl)O—; preferably R⁶ is OH or C₁-C₁₈ alkoxy; more preferably R⁶ is OH or C₁-C₆ alkoxy; even more preferably R⁶ is C₁-C₆ alkoxy.

Preferred compounds of Formula I are selected from the group of:

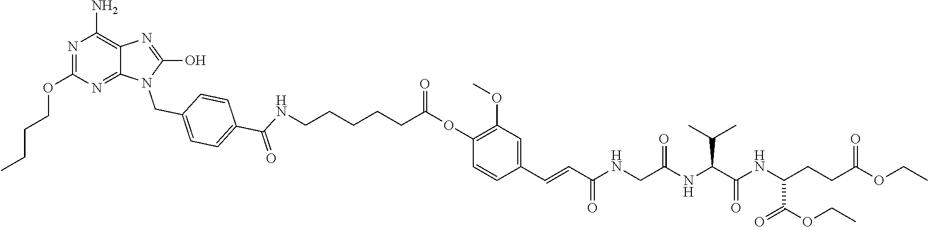 [SMILES: CCCCOc1nc(N)c2nc(O)n(Cc3ccc(C(=O)NCCCCCC(=O)Oc4ccc(/C=C/C(=O)NCC(=O)N[C@H](C(=O)N[C@H](CCC(=O)OCC)C(=O)OCC)C(C)C)cc4OC)cc3)c2n1]

Diethyl ((E)-3-(4-((6-(4-((6-amino-2-butoxy-8-hydroxy-9H-purin-9-yl)methyl)benzamido)hexanoyl)-oxy)-3-methoxyphenyl)acryloyl)glycyl-L-valyl-D-glutamate,

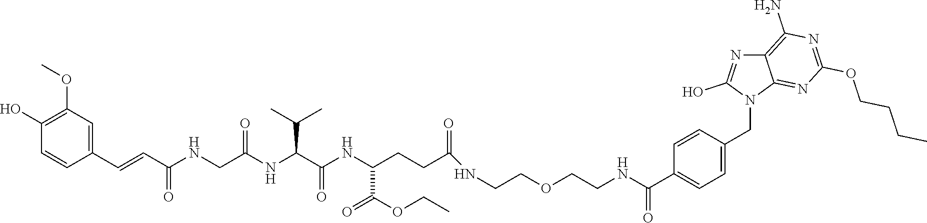 [SMILES: CCCCOc1nc(N)c2nc(O)n(Cc3ccc(C(=O)NCCOCCNC(=O)CC[C@@H](NC(=O)[C@@H](NC(=O)CNC(=O)/C=C/c4ccc(O)c(OC)c4)C(C)C)C(=O)OCC)cc3)c2n1]

Ethyl N5-(2-(2-(4-((6-amino-2-butoxy-8-hydroxy-9H-purin-9-yl)methyl)benzamido)ethoxy)ethyl)-N2-((E)-3-(4-hydroxy-3-methoxyphenyl)acryloyl)glycyl-L- valyl-D-glutaminate,

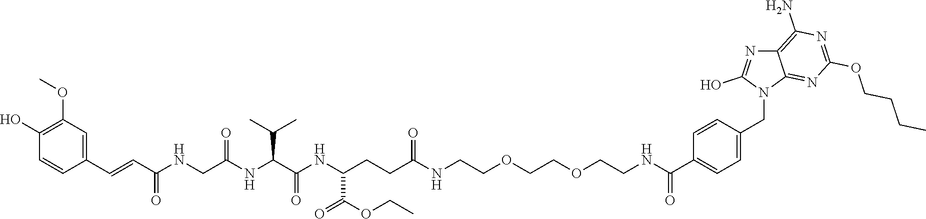 [SMILES: CCCCOc1nc(N)c2nc(O)n(Cc3ccc(C(=O)NCCOCCOCCNC(=O)CC[C@@H](NC(=O)[C@@H](NC(=O)CNC(=O)/C=C/c4ccc(O)c(OC)c4)C(C)C)C(=O)OCC)cc3)c2n1]

Ethyl (R)-1-(4-((6-amino-2-butoxy-8-hydroxy-9H-purin-9-yl)methyl)phenyl)-15-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate,

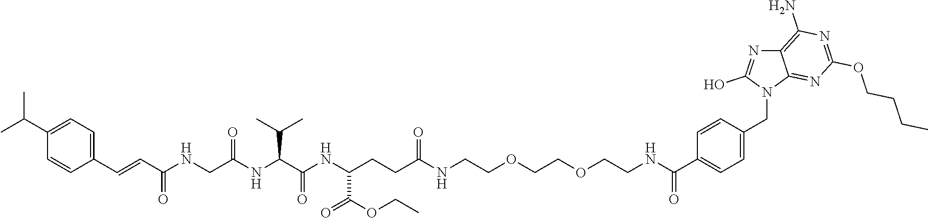 [SMILES: CCCCOc1nc(N)c2nc(O)n(Cc3ccc(C(=O)NCCOCCOCCNC(=O)CC[C@@H](NC(=O)[C@@H](NC(=O)CNC(=O)/C=C/c4ccc(C(C)C)cc4)C(C)C)C(=O)OCC)cc3)c2n1]

Ethyl (R)-1-(4-((6-amino-2-butoxy-8-hydroxy-9H-purin-9-yl)methyl)phenyl)-15-((S)-2-(2-((E)-3-(4-isopropylphenyl)acrylamido)acetamido)-3- methylbutanamido)-1,12-dioxo-5,8-dioxa-2,11-diazahexadecan-16-oate,

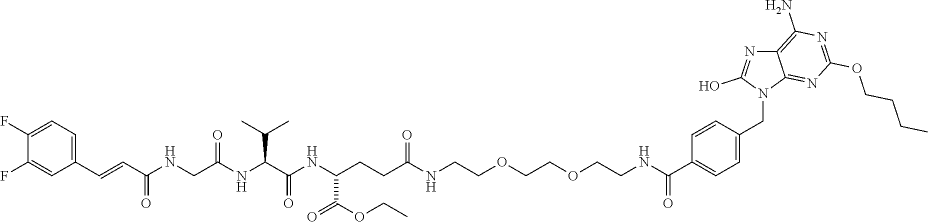 [SMILES: CCCCOc1nc(N)c2nc(O)n(Cc3ccc(C(=O)NCCOCCOCCNC(=O)CC[C@@H](NC(=O)[C@@H](NC(=O)CNC(=O)/C=C/c4ccc(F)c(F)c4)C(C)C)C(=O)OCC)cc3)c2n1]

Ethyl (R)-1-(4-((6-amino-2-butoxy-8-hydroxy-9H-purin-9-yl)methyl)phenyl)-15-((S)-2-(2-((E)-3-(3,4-difluorophenyl)acrylamido)acetamido)-3- methylbutanamido)-1,12-dioxo-5,8-dioxa-2,11-diazahexadecan-16-oate,

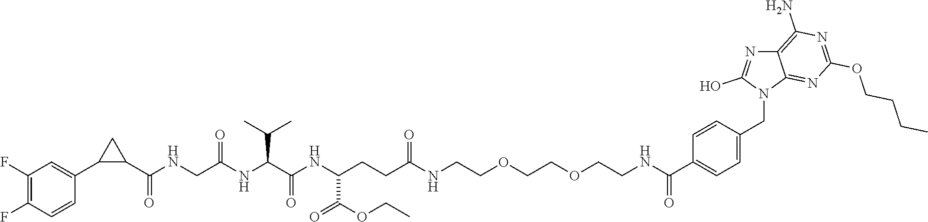 [SMILES: CCCCOc1nc(N)c2nc(O)n(Cc3ccc(C(=O)NCCOCCOCCNC(=O)CC[C@@H](NC(=O)[C@@H](NC(=O)CNC(=O)C4CC4c4ccc(F)c(F)c4)C(C)C)C(=O)OCC)cc3)c2n1]

Ethyl (15R)-1-(4-((6-amino-2-butoxy-8-hydroxy-9H-purin-9-yl)methyl)phenyl)-15-((2S)-2-(2-(2-(3,4-difluorophenyl)cyclopropane-1- carboxamido)acetamido)-3-methylbutanamido)-1,12-dioxo-5,8-dioxa-2,11-diazahexadecan-16-oate,

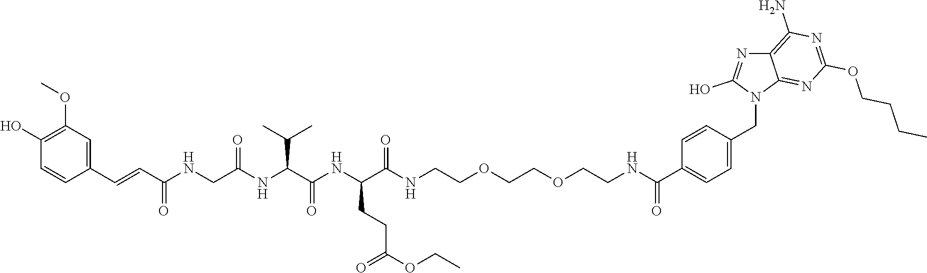 [SMILES: CCCCOc1nc(N)c2nc(O)n(Cc3ccc(C(=O)NCCOCCOCCNC(=O)[C@@H](CCC(=O)OCC)NC(=O)[C@@H](NC(=O)CNC(=O)/C=C/c4ccc(O)c(OC)c4)C(C)C)cc3)c2n1]

Ethyl (R)-1-(4-((6-amino-2-butoxy-8-hydroxy-9H-purin-9-yl)methyl)phenyl)-13-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate,

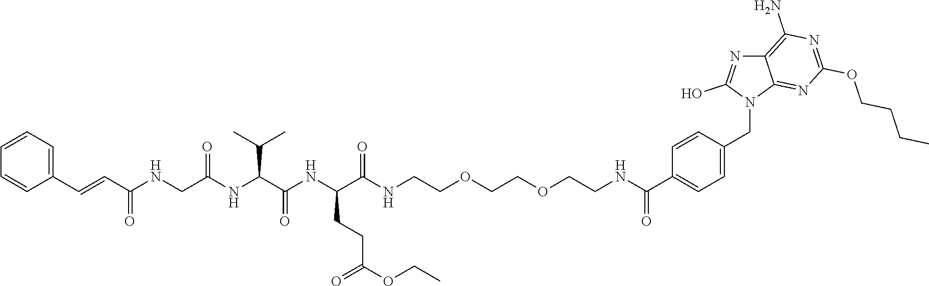 [SMILES: CCCCOc1nc(N)c2nc(O)n(Cc3ccc(C(=O)NCCOCCOCCNC(=O)[C@@H](CCC(=O)OCC)NC(=O)[C@@H](NC(=O)CNC(=O)/C=C/c4ccccc4)C(C)C)cc3)c2n1]

Ethyl (R)-1-(4-((6-amino-2-butoxy-8-hydroxy-9H-purin-9-yl)methyl)phenyl)-13-((S)-2-(2-cinnamamidoacetamido)-3-methylbutanamido)-1,12-dioxo-5,8- dioxa-2,11-diazahexadecan-16-oate,

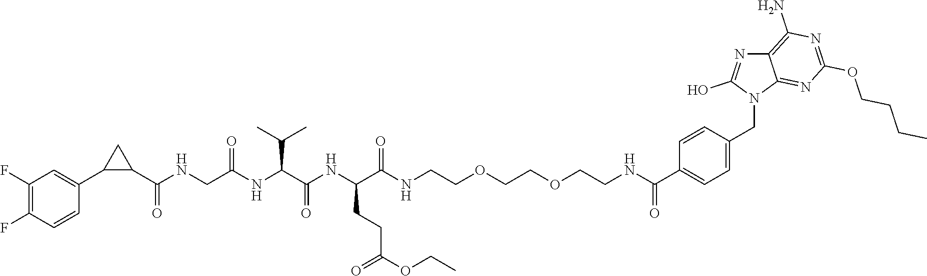 [SMILES: CCCCOc1nc(N)c2nc(O)n(Cc3ccc(C(=O)NCCOCCOCCNC(=O)[C@@H](CCC(=O)OCC)NC(=O)[C@@H](NC(=O)CNC(=O)C4CC4c4ccc(F)c(F)c4)C(C)C)cc3)c2n1]

Ethyl (13R)-1-(4-((6-amino-2-butoxy-8-hydroxy-9H-purin-9-yl)methyl)phenyl)-13-((2S)-2-(2-(2-(3,4-difluorophenyl)cyclopropane-1- carboxamido)acetamido)-3-methylbutanamido)-1,12-dioxo-5,8-dioxa-2,11-diazahexadecan-16-oate,

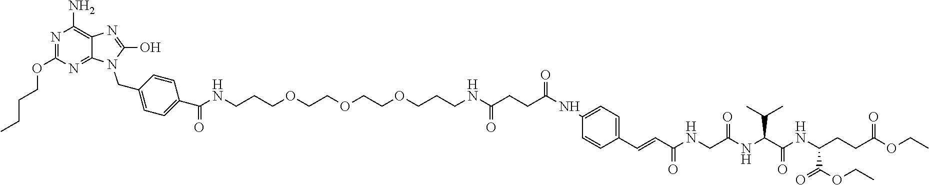

Diethyl ((E)-3-(4-(1-(4-((6-amino-2-butoxy-8-hydroxy-9H-purin-9-yl)methyl)phenyl)-1,17-dioxo-6,9,12-trioxa-2,16-diazaicosan-20- amido)phenyl)acryloyl)glycyl-L-valyl-D-glutamate,

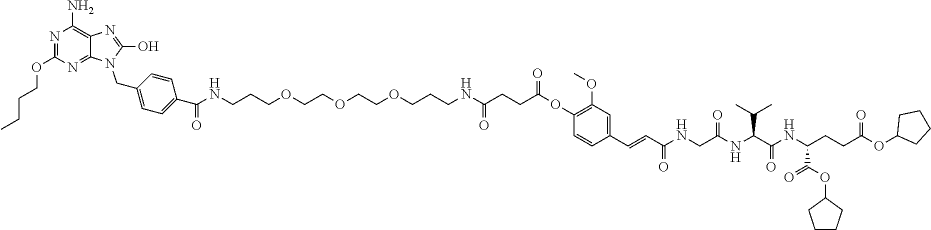

Dicyclopentyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3-methoxyphenyl)acryloyl)glycyl-L-valyl-D-glutamate,

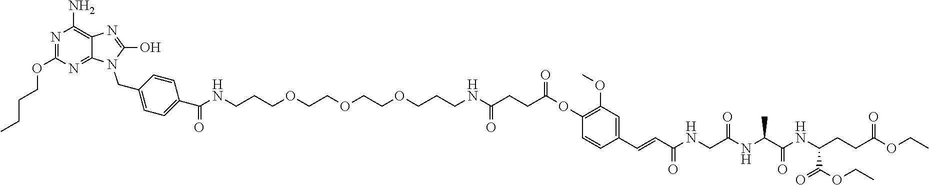

Diethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3- methoxyphenyl)acryloyl)glycyl-L-alanyl-D-glutamate,

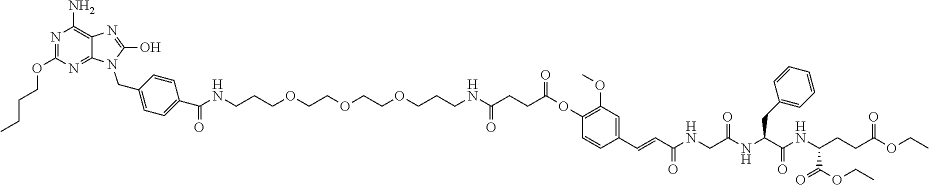

Diethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3- methoxyphenyl)acryloyl)glycyl-L-phenylalanyl-D-glutamate, and

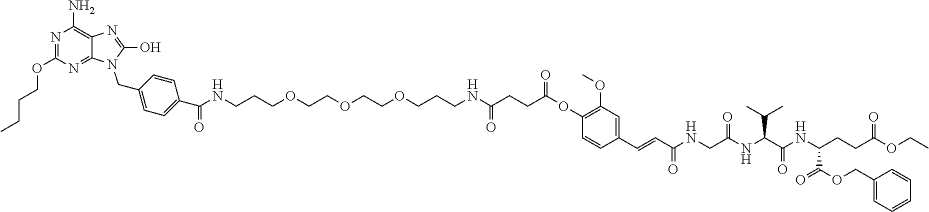

1-benzyl 5-ethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3-methoxyphenyl)acryloyl)glycyl-L-valyl-D-glutamate.

### Linking Group

In certain embodiments of the invention, L is a linking group that functions to covalently connect a TLR7 agonist of formula

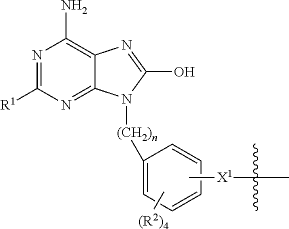

to a NOD2 agonist selected from the group of

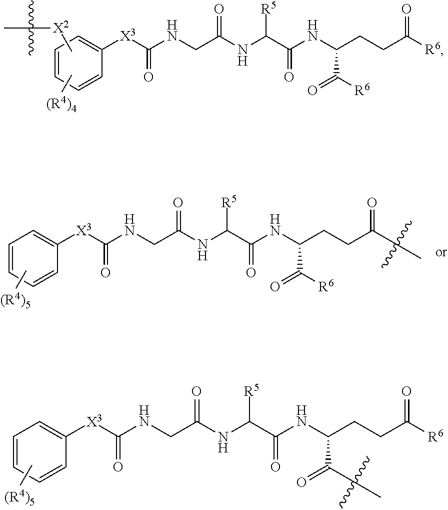

wherein R¹, R², R³, R⁴, R⁵, R⁶, X¹, X², X³ and n are as defined previously.

The specific composition of the linking group L is not critical provided the resulting conjugate retains the useful biological properties described herein. Linker type and length can be readily optimized in the context of the other substituents in the conjugated compound by using the assays provided in EXAMPLE 17. The selection of a linker component is based on its documented properties of biocompatibility and solubility in aqueous and organic media.

According to certain embodiments, the linker L is a non-peptidic polymeric linker. Non-limiting examples of suitable non-peptidic polymeric linkers include polyalkylene oxides (e.g. polyethylene glycol, polypropylene glycol, and the like), polyvinyl alcohol, polyvinylpyrrolidone, and the like, as well as derivative and copolymers thereof.

According to certain embodiments, the linker L comprises a polyethylene glycol (PEG) chain. According to certain embodiments, the polyethylene glycol chain comprises from 2 to 100, such as 2 to 50, repeating ethylene glycol units. According to certain embodiments, the polyethylene glycol chain comprises 2, 3, 4, 5, 6, 7, 8, 9 or 10 repeating ethylene glycol units. According to certain embodiments, one or both terminal hydroxy groups on the polyethylene glycol chain may be substituted with groups selected from amine, thiol, azide, carboxy, hydroxyl, N-hydroxysuccinimide and maleimide.

According to certain embodiments, the linker L is non-polymeric aliphatic linker, comprising of a divalent, linear or branched, straight or cyclic, saturated or unsaturated, hydrocarbon chain, having from 2 to 100 carbon atoms, wherein the carbon atoms are optionally replaced by a group selected from —O—, —S—, —NH—, —C(═O)—, —OC(═O)—, —N(C₁-C₆ alkyl)—, —NHC(═O)—, —N(C₁-C₆ alkyl)C(═O)—, —S(═O)— or —S(═O) 2- and wherein the chain is optionally substituted on carbon with one or more (e.g. 1, 2, 3 or 4) substituents. The non-polymeric aliphatic linkers are typically derived from an aliphatic compound having at least two functional groups, capable of reacting with functional groups on the TLR7 and NOD2 agonist moieties (e.g. carboxy, NH₂, OH, and the like).

According to certain embodiments, the linker L is a divalent radical formed from an amino acid. According to certain embodiments, the linker L is a divalent radical formed from a natural amino acid and stereoisomers thereof.

According to certain embodiments, the linker L is a divalent radical formed from a peptide. According to certain embodiments, the peptide includes naturally occurring amino acids, and stereoisomers thereof. According to certain embodiments, the peptide is formed only from naturally occurring amino acids, and stereoisomers thereof. According to certain embodiments, the linker L is a polyproline linker. According to certain embodiments, the linker L is a polyglycine linker.

According to certain embodiments, the linker L is —NH—(CH₂)₅—C(═O)—.

According to certain embodiments, the linker L is —NH—(CH₂)₂—O—(CH₂)₂—NH—.

According to certain embodiments, the linker L is —NH—(CH₂)₂—O—(CH₂)₂—O—(CH₂)₂—NH—.

According to certain embodiments, the linker L is —NH—(CH₂)₃—O—(CH₂)₂—O—(CH₂)₂—O—(CH₂)₃—NH—C(═O)—(CH₂)₂—C(═O)—.

### Therapeutic Applications

Conjugated compounds of the present invention are TLR7 and NOD2 agonists. Thus, compounds of Formula I or pharmaceutically acceptable salts, racemates, diastereomers, enantiomers, esters or prodrugs thereof are useful in the treatment of conditions for which agonism of TLR7 and NOD2 is beneficial.

Immune responses generated by simultaneous activation of both TLR7 and NOD2 lead to the production and activation of pro-inflammatory cytokines. Thus, in one aspect, conjugated compounds of the invention are useful for the treatment of viral, bacterial, fungal and protozoal infections, tumors or cancers and immunological diseases.

According to certain embodiments, a compound of the invention is useful for the treatment of a viral, bacterial, fungal or protozoal infection. According to certain embodiments, a compound of the invention is useful for the treatment of a viral infection. According to certain embodiments, a compound of the invention is useful for the treatment of a bacterial infection. According to certain embodiments, a compound of the invention is useful for the treatment of a fungal infection. According to certain embodiments, a compound of the invention is useful for the treatment of a protozoal infection.

According to certain embodiments, a compound of the invention is useful for the treatment of cancer.

According to certain embodiments, a compound of the invention is useful for the treatment of an immunological disease.

In another aspect, the conjugated compounds of the invention are useful as vaccine adjuvants. Accordingly, this specification discloses a compound of Formula I or a pharmaceutically acceptable salt, racemate, diastereomer, enantiomer, ester or prodrug thereof for use in medicine, for immune modulation for the treatment of a disease.

Compounds of the present invention can be formulated as pharmaceutical compositions and administered to a subject, such as a human patient in a variety of forms adapted to the chosen route of administration, i.e. orally or parenterally, by intravenous, intramuscular, topical or subcutaneous routes. Pharmaceutical compositions comprising a compound of Formula I, may be prepared using techniques and methods known to those skilled in the art. Some of the methods commonly used in the art are described in Remington's The Science and Practice of Pharmacy.

Accordingly, one aspect of the invention is directed to pharmaceutical compositions comprising a compound of Formula I and one or more pharmaceutically acceptable excipients. For example, the conjugated compounds of the invention can be formulated for parenteral administration, such as intravenous administration or administration into a body cavity or lumen of an organ. Formulations for injection will commonly comprise a solution of the conjugated compound of the invention dissolved in a pharmaceutically acceptable carrier. Among the acceptable vehicles and solvents that can be employed are water and Ringer's solution. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

These compositions can also be used as vaccine adjuvants. Thus, another aspect of the present invention is a vaccine comprising the conjugated compound of the invention.

Used as vaccine adjuvants, the compounds of Formula I and pharmaceutical compositions thereof can be administered at the same time and by the same method as the antigen (viral, bacterial, parasitic antigen and the like) against which it is desired to increase the cell immunity reactions (type IV hypersensitivity) or the production of circulating or local antibodies in the immunized subject.

Compounds of the present invention may also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in lipid form can contain, in addition to a compound of the present invention, stabilizers, preservatives, excipients, and the like. Typical lipids are the phospholipid and phosphatidyl choline, both natural and synthetic. Methods of forming liposomes are known in the art and are described in Prescott's Methods in Cell Biology, which is incorporated herein by reference.

### Process

Another aspect of the present invention is the process for the manufacture of compounds of Formula I or a pharmaceutically acceptable salt thereof.

If not commercially available, the necessary starting materials for the procedures such as those described below may be made by procedures which are selected from standard organic chemistry techniques, techniques which are analogous to the synthesis of knows structurally similar compounds, or techniques, which are analogous to the procedures described in the examples.

It will also be appreciated that in some of the reactions mentioned herein it may be necessary/desirable to protect any sensitive groups in compounds. The instances where protection is necessary or desirable are known to those skilled in the art, as are suitable methods for such protection.

Example of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanol or an aroyl group may be removed, for example, by hydrolysis with a suitable base such as an alkali metal hydroxide, for example lithium of sodium hydroxide.

Alternatively, a silyl group such as trimethylsilyl may be removed, for example, by fluoride or by aqueous acid; or an arylmethyl group such as a benzyl group may be removed, for example, by hydrogenation in the presence of catalyst such as palladium-on-carbon.

A suitable protecting group for an amino group is, for example, any acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid such as hydrochloric, sulphuric, phosphoric or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron tris (trifluoroacetate). A suitable alternative protecting group for a primary amino group is, for example, a phthalogenyl group which may be removed by treatment with an alkylamine, for example dimethylaminopropylamine or 2-hydroxyethylamine, or with hydrazine.

Example of a suitable protecting group for a carboxy group is, for example, an alkyl group, for example a methyl, ethyl or t-butyl group or an aryl group, for example a benzyl group. The deprotection conditions for the above protecting groups necessarily vary with the choice of protecting group. Thus, for example, an alky group such as a methyl or ethyl group may be removed for example, by hydrolysis with a suitable base such as an alkali metal hydroxide, for example lithium or sodium hydroxide. Alternatively, an alkyl group such as a t-butyl group may be removed, for example, by treatment with a suitable acid such as hydrochloric, sulphuric, phosphoric or trifluoroacetic acid. An aryl group such as a benzyl group may be removed, for example, by hydrogenation over a catalyst such as palladium-on-carbon.

Other suitable protecting groups well known to those skilled in the art can be found in “Protective Groups in Organic Synthesis”, 3^{rd} Ed. by Greene and Wuts (John Wiley and Sons, 1999), incorporated herein by reference.

The protecting groups may be removed at any convenient stage in the synthesis using conventional techniques well known in the chemical art, or they may be removed during a later reaction step or work-up.

Thus, the present invention also provides that the compounds of the Formula I and pharmaceutically acceptable salts thereof can be prepared by a process comprising reacting a compound of Formula V:

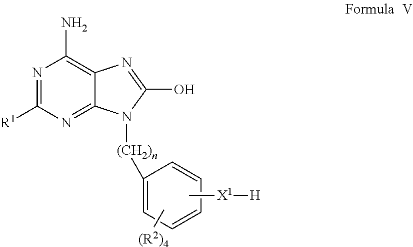

wherein R¹, R², X¹ and n are as defined previously,
with a compound of Formula VI:

H—L—R³ Formula VI,

wherein R³ and L are as defined previously;
or a compound of Formula VII:

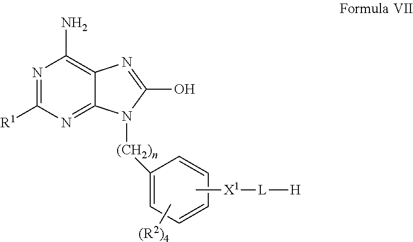

wherein R¹, R², X¹, L and n are as defined previously,
with a compound of Formula VIII:

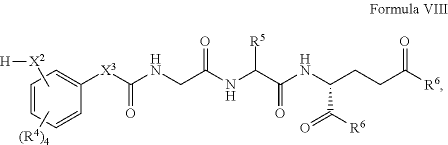

### Formula IX:

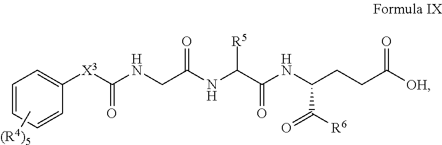

### or Formula X:

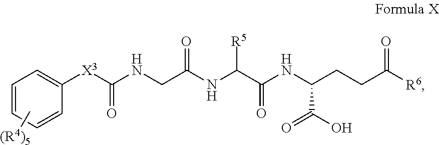

wherein R⁴, R⁵, R⁶, X² and X³ are as defined previously;
and optionally thereafter carrying out one or more of the following procedures:
removing any protecting groups,
forming a pharmaceutically acceptable salt,
converting a compound of Formula I into another compound of Formula I.

Provided by these processes are conjugated compounds of Formula XI and pharmaceutically acceptable salts thereof:

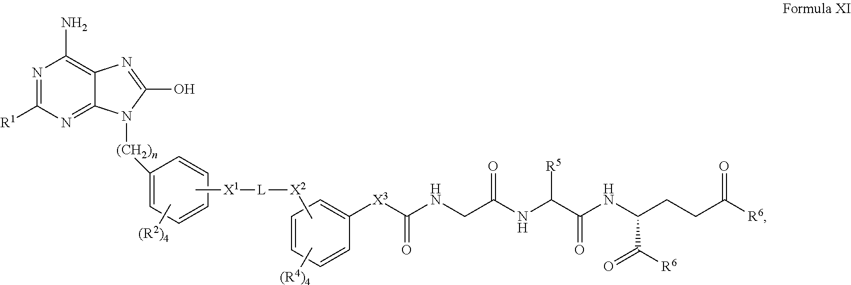

### Formula XII:

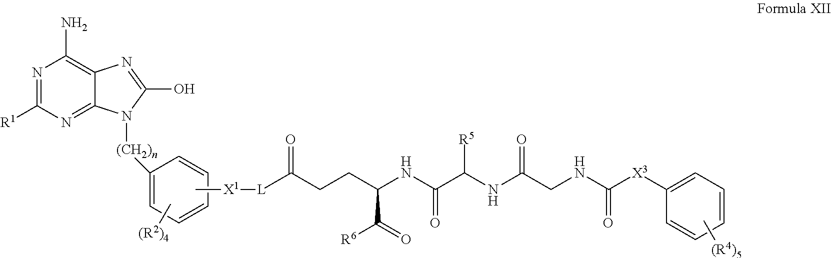

### or Formula XIII:

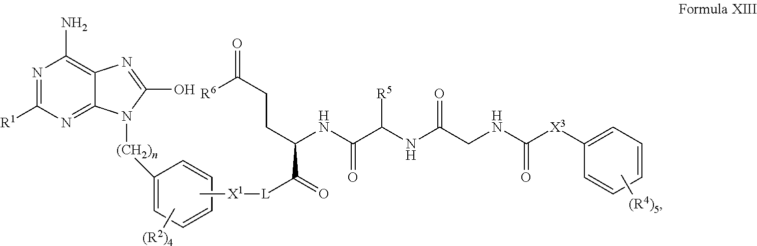

wherein R¹, R², R⁴, R⁵, R⁶, X¹, X², X³, L and n are as defined previously.

### EXAMPLES

### Methods of Synthesis

The present invention is further exemplified, but not limited by, the following examples that illustrate the preparation of compounds of Formula I. The use of these and other examples anywhere in the specification is illustrative only, and in no way limits the scope and meaning of the invention or of any exemplified form. Likewise, the invention is not limited to any particular preferred embodiments described herein. Indeed, many modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

Unless otherwise stated:
evaporation was carried out by rotary evaporation in vacuo and work-up procedures were carried out after removal of residual solids after filtration;
operations were generally carried out at ambient temperature, that is typically between 18 and 26° C. and without exclusion of air unless otherwise stated, or a person skilled in the art would otherwise work under an inert atmosphere;
flash column chromatography was used to purify compounds and was performed on Merck Silica Gel 60 unless otherwise stated;
yields are given for illustration only and are not necessarily the maximum attainable;
the structure of the end-products was generally confirmed by NMR and mass spectral techniques; proton NMR spectra is quoted and was determined using a Bruker Avance III 400 MHz spectrometer operating at field strength of 400 MHz. Chemical shifts are reported in part per million downfield from tetramethylsilane as an internal standard and peak multiplicities are shown thus: s, singlet; brs, broad singlet; d, doublet; t, triplet; q, quartet; m, multiplet
mass spectra were obtained using a Q TOF Premier mass spectrometer (Micromass, Waters, Manchester, UK) and Exactive Plus Orbitrap mass spectrometer (Thermo Fisher Scientific, Waltham, Massachusetts, USA)
each intermediate was generally purified to the standard required for the subsequent stage and was characterized in sufficient detail to confirm that the assigned structure was correct; purity was assessed by high pressure liquid chromatography, thin layer chromatography, or NMR and identity was determined by mass spectrometry and NMR spectroscopy as appropriate.

### Abbreviations

As used herein, the symbols and conventions are consistent with those used in the contemporary scientific literature. Specifically, the following abbreviations are used in the text:
Boc₂O for di-tert-butyl decarbonate; CDCl₃ for deuterated chloroform; COMU for (1-cyano-2-ethoxy-2-oxoethylidenaminooxy)dimethylamino-morpholino-carbenium hexafluorophosphate; DCC for N,N′-dicyclohexylcarbodiimide; DCM for dichloromethane; DIPEA for N,N-diisopropylethylamine; DMAP for 4-dimethylaminopyridine; DMF for dimethylformamide; DMSO for dimethyl sulfoxide; DMSO-d₆ for deuterated dimethyl sulfoxide; EDC for N-(3-dimethylaminopropyl)-N′-ethylcarbodiimide hydrochloride; eq for equivalent; g for gram; HATU for 1-[bis(dimethylamino)methylene]-1H-1,2,3-triazolo[4,5-b]pyridinium 3-oxid hexafluorophosphate; HCl for hydrochloric acid; HOBt for hydroxybenzotriazole; K₂CO₃ for potassium carbonate; LPS for lipopolysaccharides; mL for milliliter; mmol for millimole; NaHCO₃ for sodium hydrogen carbonate; NaOH for sodium hydroxide; Na₂SO₄ for sodium sulphate; Na₂S₂O₃ for sodium thiosulfate; NH₄ Cl for ammonium chloride; NMR for nuclear magnetic resonance; PBS for phosphate buffered saline; RPM for revolutions per minute; rt for room temperature; TEA for triethylamine; TFA for trifluoroacetic acid

### Example 1: Compound SG8

### Intermediate 1

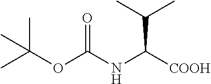

(tert-Butoxycarbonyl)-L-valine: L-valine (3.515 g, 30 mmol, 1 eq) was dissolved in water (10 mL) and 1M aqueous NaOH (36 mL). Boc₂ O (8.512 g, 39 mmol, 1.3 eq) was dissolved in dioxane (30 mL) and added dropwise to the ice-chilled stirring solution of L-valine. The resulting mixture was stirred at rt for 20 h. After evaporating dioxane in vacuo, the mixture was washed with diethyl ether. After acidifying the mixture with a 1M HCl solution it was extracted three times with ethyl acetate (50 mL). The combined organic phases were dried over Na₂SO₄ and concentrated in vacuo to afford the subject compound as a colourless oil (6.518 g, yield 100%). ¹H NMR (400 MHz, DMSO-d₆) δ12.46 (s, 1H), 6.94 (d, 1H), 3.82-3.75 (m, 1H), 2.06-1.94 (m, 1H), 1.39 (s, 9H), 0.87 (t, 6H).

### Intermediate 2

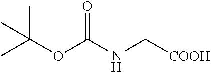

(tert-Butoxycarbonyl)glycine: Glycine (2.252 g, 30 mmol, 1 eq) was dissolved in water (10 mL) and 1M aqueous NaOH (36 mL). Boc₂O (8.512 g, 39 mmol, 1.3 eq) was dissolved in dioxane (30 mL) and added dropwise to the ice-chilled stirring solution of glycine. The resulting mixture was stirred at rt for 20 h. After evaporating dioxane in vacuo, the mixture was washed with diethyl ether. After acidifying the mixture with a 1M HCl solution it was extracted three times with ethyl acetate (50 mL). The combined organic phases were dried over Na₂SO₄ and concentrated in vacuo to afford the subject compound as a colourless oil (5.255 g, yield 100%). 1 H NMR (400 MHz, DMSO—d 6) δ12.46 (s, 1H), 7.08 (t, 1H), 3.59-3.56 (m, 2H), 1.38 (s, 9H).

Intermediate 3

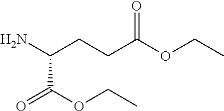

Diethyl D-glutamate: To an ice-chilled stirring suspension of D-glutamic acid (2.943 g, 20 mmol, 1 eq) in absolute ethanol (40 mL) was added thionyl chloride (3.20 mL, 44 mmol, 2.2 eq). The resulting mixture was refluxed with stirring for 20 h. After concentrating the mixture in vacuo, the residue was precipitated in diethyl ether. Diethyl ether was evaporated in vacuo to afford the subject compound as a white crystalline powder (4.794 g, yield 100%), which was used in the next step without any further purification.

Intermediate 4

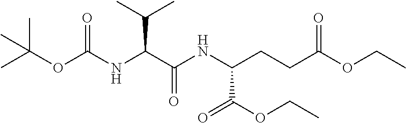 [SMILES: CCOC(=O)CC[C@@H](NC(=O)[C@@H](NC(=O)OC(C)(C)C)C(C)C)C(=O)OCC]

Diethyl (tert-butoxycarbonyl)-L-valyl-D-glutamate: To an ice-chilled stirring solution of Intermediate 1 (1.673 g, 7.7 mmol, 1.1 eq) in ethyl acetate (50 mL) were added HOBt (1.040 g, 7.7 mmol, 1.1 eq) and DCC (1.589 g, 7.7 mmol, 1.1 eq). After stirring for 30 minutes, Intermediate 3 (1.678 g, 7 mmol, 1 eq), TEA (2.44 mL, 17.5 mmol, 2.5 eq) and DMAP (catalytic amount) were added and the resulting mixture was stirred at rt for 22 h, after which hexane (20 mL) was added. The mixture was filtered and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as an off-white crystalline solid (2.457 g, yield: 87%), which was used in the next step without any further purification.

Intermediate 5

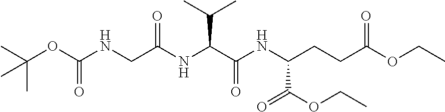 [SMILES: CCOC(=O)CC[C@@H](NC(=O)[C@@H](NC(=O)CNC(=O)OC(C)(C)C)C(C)C)C(=O)OCC]

Diethyl (tert-butoxycarbonyl)glycyl-L-valyl-D-glutamate: Intermediate 4 (2.424 g, 6.02 mmol) was added to an ice-chilled stirred mixture of TFA and DCM (1/5, 24 mL), and the mixture was allowed to warm to room temperature. After 3 h, the solvent was evaporated in vacuo. The residue was coevaporated three times with diethyl ether. Concurrently, HOBt (0.894 g, 6.62 mmol, 1.1 eq), DCC (1.366 g, 6.62 mmol, 1.1 eq) and DMAP (catalytic amount) were added to an ice-chilled stirring solution of Intermediate 2 (1.16 g, 6.62 mmol, 1.1 eq) in ethyl acetate (30 mL). After 30 minutes, a solution of the deprotected Intermediate 4 in ethyl acetate (20 mL) and TEA (4.3 mL, 30.1 mmol, 5 eq) was added and the resulting mixture was stirred at rt for 18 h. Hexane (20 mL) was added, after which the mixture was filtered and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as an off-white crystalline solid (2.06 g, yield: 75%). ¹H NMR (400 MHz, DMSO-d₆) δ8.41 (d, 1H), 7.58 (d, 1H), 7.03 (t, 1H), 4.28-4.23 (m, 2H), 4.11-4.01 (m, 4H), 3.58 (d, 2H), 2.36 (t, 2H), 2.04-1.92 (m, 2H), 1.87-1.78 (m, 1H), 1.39 (s, 9H), 1.20-1.16 (m, 6H), 0.86-0.81 (m, 6H).

### Compound 6

### SG8

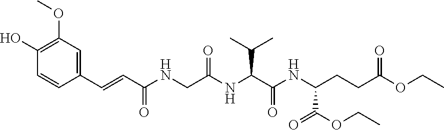 [SMILES: CCOC(=O)CC[C@@H](NC(=O)[C@@H](NC(=O)CNC(=O)/C=C/c1ccc(O)c(OC)c1)C(C)C)C(=O)OCC]

Diethyl ((E)-3-(4-hydroxy-3-methoxyphenyl)acryloyl)glycyl-L-yalyl-D-glutamate: Intermediate 5 (0.919 g, 2 mmol, 1 eq) was added to an ice-chilled stirred mixture of TFA and DCM (1/5, 12 mL), and the mixture was allowed to warm to room temperature. After 3 h, the solvent was evaporated in vacuo. The residue was coevaporated three times with diethyl ether and dissolved in DMF (6 mL). After cooling the solution in ice, TEA (1.4 mL, 10 mmol, 5 eq), trans-ferulic acid (0.427 g, 2.2 mmol, 1.1 eq), HOBt (0.297 g, 2.2 mmol, 1.1 eq), DMAP (catalytic amount) and EDC (0.422 g, 2.2 mmol, 1.1 eq) were added and the resulting mixture was stirred at rt for 18 h. The reaction mixture was diluted with ethyl acetate (60 mL) and washed with a 1 M HCl solution (2×30 mL), saturated NaHCO₃ solution (2×30 mL) and brine (30 mL). The organic layer was dried over anhydrous Na₂SO₄ and concentrated in vacuo to afford the subject compound as an orange solid (0.804 g, yield: 75%). ¹H NMR (400 MHz, DMSO-d₆) δ9.46 (s, 1H), 8.39 (d, 1H), 8.20 (t, 1H), 7.90 (d, 1H), 7.33 (d, 1H), 7.14 (s, 1H), 7.00 (d, 1H), 6.79 (d, 1H), 6.56 (d, 1H), 4.28-4.22 (m, 2H), 4.10-3.99 (m, 4H), 3.88 (d, 2H), 3.81 (s, 3H), 2.35 (t, 2H), 2.04-1.92 (m, 2H), 1.88-1.79 (m, 1H), 1.19-1.13 (m, 6H), 0.85 (t, 6H).

### Example 2: Compound SG43

### Intermediate 7

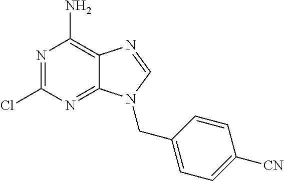 [SMILES: N#Cc1ccc(Cn2cnc3c(N)nc(Cl)nc32)cc1]

4-((6-amino-2-chloro-9H-purin-9-yl)methyl)benzonitrile: 2-chloroadenine (1.017 g, 6 mmol, 1.0 eq), potassium carbonate (2.579 g, 18.66 mmol 3.11 eq) and 4-(bromomethyl)benzonitrile (1.625 g, 8.29 mmol 1.38 eq) were suspended in DMSO (22 mL) and stirred at rt for 20 h. The reaction mixture was poured into ethyl acetate (130 mL) and water (90 mL). After mixing thoroughly, the mixture was concentrated in vacuo. The concentrated mixture was cooled in ice and the formed precipitate was filtered, washed with cold water and dried to afford the subject compound (1.737 g, yield: 89%). ¹H NMR (400 MHz, DMSO-d₆) δ8.28 (s, 1H), 7.85-7.83 (m, 4H), 7.42 (d, 2H), 5.45 (s, 2H).

### Intermediate 8

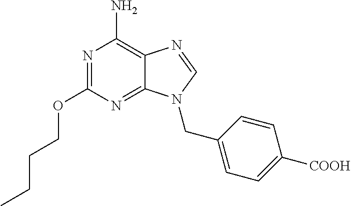 [SMILES: CCCCOc1nc(N)c2ncn(Cc3ccc(C(=O)O)cc3)c2n1]

4-((6-amino-2-butoxy-9H-purin-9-yl)methyl)benzoic acid: Intermediate 7 (1.737 g, 6.10 mmol, 1 eq) was suspended in dry n-butanol (60 mL). A 20% solution of sodium n-butoxide in n-butanol (33.6 mL, 61.01 mmol, 10 eq) was added and the resulting mixture was refluxed with stirring for 20 h. The reflux was paused to cool the mixture. Water (20 mL) was added and the reflux was continued for additional 20 h. The reaction mixture was extracted three times with 80 mL of water. The combined aqueous layers were acidified to pH 3 with concentrated HCl and cooled overnight. The white precipitate obtained was filtered and dried to afford the subject compound (1.605 g, yield: 77%). ¹H NMR (400 MHz, DMSO-d₆) δ12.69 (br s, 1H), 8.07 (s, 1H), 7.91 (d, 2H), 7.39 (d, 2H), 7.25 (s, 2H), 5.35 (s, 2H), 4.20-4.18 (m, 2H), 1.64-1.61 (m, 2H), 1.39-1.35 (m, 2H), 0.90 (t, 3H).

### Intermediate 9

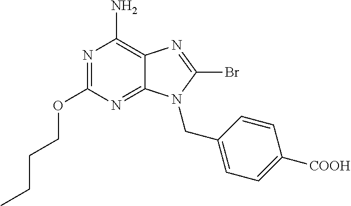 [SMILES: CCCCOc1nc(N)c2nc(Br)n(Cc3ccc(C(=O)O)cc3)c2n1]

4-((6-amino-8-bromo-2-butoxy-9H-purin-9-yl)methyl)benzoic acid: Intermediate 8 (1.605 g, 4.701 mmol, 1 eq) was suspended in acetic acid (60 mL). Sodium acetate (1.928 g, 23.51 mmol, 5 eq) and bromine (1.22 mL, 23.74 mmol, 5.05 eq) were added and the resulting mixture was stirred at rt for 2 h. Aqueous Na₂S₂O₃ was added to the reaction mixture. The precipitate obtained was filtered and washed with cold water and diethyl ether to afford the subject compounds as a yellow powder (1.976 g, yield 100%). ¹H NMR (400 MHz, DMSO-d₆) δ7.92 (d, 2H), 7.44 (brs, 2H), 7.30 (d, 2H), 5.33 (s, 2H), 4.19 (t, 2H), 1.66-1.59 (m, 2H), 1.40-1.35 (m, 2H), 0.91 (t, 3H).

### Compound 10

### SG43

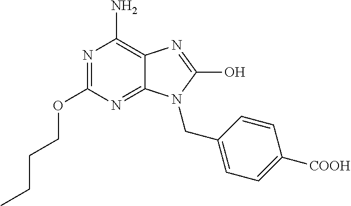 [SMILES: CCCCOc1nc(N)c2nc(O)n(Cc3ccc(C(=O)O)cc3)c2n1]

4-((6-amino-2-butoxy-8-hydroxy-9H-purin-9-yl)methyl)benzoic acid: To Intermediate 9 (1.976 g, 4.701 mmol) in methanol (35 mL) was added 10 M aqueous NaOH (35 mL). The mixture was refluxed with stirring for 24 h. The solution was cooled to room temperature and acidified with 6 M HCl solution. After concentrating the mixture in vacuo, the off-white precipitate obtained was filtered and washed with water and diethyl ether to afford the subject compound (1.523 g, yield 91%). ¹H NMR (400 MHz, DMSO-d₆) δ10.25 (s, 1H), 7.89 (d, 2H), 7.38 (d, 2H), 6.63 (brs, 2H), 4.93 (s, 2H), 4.13 (t, 2H), 1.62-1.58 (m, 2H), 1.38-1.33 (m, 2H), 0.89 (t, 3H).

### Example 3: Compound SG48

### Intermediate 11

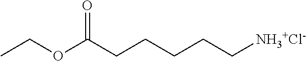

6-ethoxy-6-oxohexan-1-aminium chloride: To 6-aminohexanoic acid (656 mg, 5 mmol, 1 eq) in ethanol (5 mL) was added thionyl chloride (0.55 mL, 7.5 mmol, 1.5 eq) and refluxed with stirring for 3 h. The reaction mixture was concentrated in vacuo and coevaporated with diethyl ether to afford the subject compound as a white solid (0.976 g, yield 100%). ¹H NMR (400 MHz, DMSO-d₆) δ8.00 (s, 3H), 4.05 (q, 2H), 2.74 (t, 2H), 2.29 (t, 2H), 1.59-1.49 (m, 4H), 1.34-1.29 (m, 2H), 1.18 (t, 3H).

### Intermediate 12

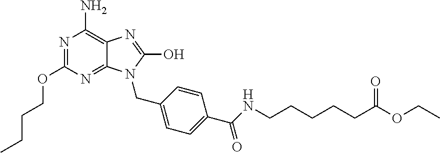 [SMILES: CCCCOc1nc(N)c2nc(O)n(Cc3ccc(C(=O)NCCCCCC(=O)OCC)cc3)c2n1]

Ethyl 6-(4-((6-amino-2-butoxy-8-hydroxy-9H-purin-9-yl)methyl)benzamido)hexanoate: Compound (185 mg, 0.518 mmol, 1 eq) was dissolved in DMSO (7 mL). Intermediate 11 (304 mg, 1.553 mmol, 3 eq) and TEA (0.576 mL, 4.141 mmol, 8 eq) were dissolved in DCM (2 mL) and added to the stirring solution of Compound 10 in DMSO. After cooling the reaction mixture in ice, COMU (554 mg, 1.295 mmol, 2.5 eq) was added and the mixture was stirred at rt for 20 h. Ethyl acetate (60 mL) and 1 M NaHCO₃ solution (40 mL) were added. After concentrating the mixture in vacuo, the mixture was cooled in ice for one hour. The precipitate was filtered and washed with water and diethyl ether to afford the subject compound (119 mg, yield: 46%). ¹H NMR (400 MHz, DMSO-d₆) δ9.99 (s, 1H), 8.40 (t, 1H), 7.76 (d, 2H), 7.34 (d, 2H), 6.48 (s, 2H), 4.90 (s, 2H), 4.21-4.09 (m, 2H), 4.02 (q, 2H), 3.22 (q, 2H), 2.27 (t, 2H), 1.66-1.56 (m, 2H), 1.55-1.45 (m, 4H), 1.41-1.21 (m, 4H), 1.15 (t, 3H), 0.90 (t, 3H).

### Intermediate 13

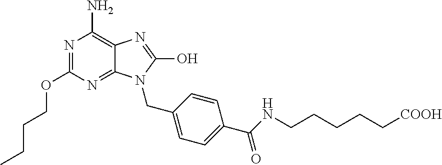 [SMILES: CCCCOc1nc(N)c2nc(O)n(Cc3ccc(C(=O)NCCCCCC(=O)O)cc3)c2n1]

6-(4-((6-amino-2-butoxy-8-hydroxy-9H-purin-9-yl)methyl)benzamido)hexanoic acid: To a stirring solution of Intermediate 12 (95 mg, 0.191 mmol) in methanol (15 mL) was added 1M aqueous NaOH (3 mL). The mixture was stirred at rt for 20 h. Water (12 mL) was added and the mixture was acidified using a 1M HCl solution. Methanol was evaporated in vacuo and the resulting suspension was cooled in ice for 1 h. The obtained precipitate was filtered and washed with water and diethyl ether to afford the subject compound as an off-white solid (59 mg, yield 66%). ¹H NMR (400 MHz, DMSO-d₆) δ12.02 (brs, 1H), 10.12 (s, 1H), 8.40 (t, 1H), 7.77 (d, 2H), 7.34 (d, 2H), 6.54 (s, 2H), 4.90 (s, 2H), 4.18-4.11 (m, 2H), 3.22 (q, 2H), 2.20 (t, 2H), 1.64-1.57 (m, 2H), 1.55-1.47 (m, 4H), 1.41-1.23 (m, 4H), 0.90 (t, 3H).

### Compound 14

### SG48

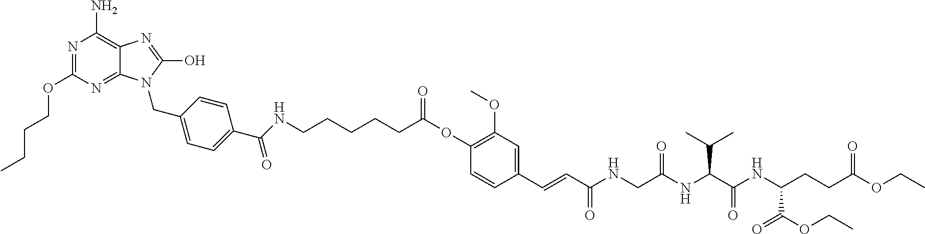 [SMILES: CCCCOc1nc(N)c2nc(O)n(Cc3ccc(C(=O)NCCCCCC(=O)Oc4ccc(/C=C/C(=O)NCC(=O)N[C@H](C(=O)N[C@H](CCC(=O)OCC)C(=O)OCC)C(C)C)cc4OC)cc3)c2n1]

Diethyl ((E)-3-(4-((6-(4-((6-amino-2-butoxy-8-hydroxy-9H-purin-9-yl)methyl)benzamido)hexanoyl)-oxy)-3-methoxyphenyl)acryloyl)glycyl-L-yalyl-D-glutamate: To an ice- chilled stirring solution of Intermediate 13 (55 mg, 0.117 mmol, 1 eq) in DMF (2 mL) were added DIPEA (61 μL, 0.351 mmol, 3 eq), Compound 6 (63 mg, 0.117 mmol, 1 eq) and COMU (55 mg, 0.129 mmol, 1.1 eq). The resulting mixture was stirred at rt for 4 h, after which it was diluted with ethyl acetate (30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo. The crude compound was purified using silica gel chromatography (7% MeOH/DCM) to afford the subject compound as an orange solid (14 mg, yield: 12%). ¹H NMR (400 MHz, DMSO-d₆) δ10.00 (s, 1H), 8.46-8.37 (m, 2H), 8.36-8.29 (m, 1H), 7.96 (d, 1H), 7.79 (d, 2H), 7.43 (d, 1H), 7.38-7.30 (m, 3H), 7.16 (d, 1H), 7.10 (d, 1H), 6.77 (d, 1H), 6.48 (s, 2H), 4.91 (s, 2H), 4.31-4.23 (m, 2H), 4.17-3.98 (m, 6H), 3.92 (d, 2H), 3.79 (s, 3H), 3.30-3.22 (m, 2H), 2.40-2.31 (m, 2H), 2.04-1.95 (m, 4H), 1.92-1.79 (m, 1H), 1.69-1.50 (m, 6H), 1.41-1.31 (m, 4H), 1.22-1.11 (m, 6H), 0.94-0.82 (m, 9H). HRMS calculated for C₄₉H₆₆O₁₃N₉ m/z: 988.4775 (M+H)⁺, found 988.4779.

### Example 4: Compound SG144

### Intermediate 15

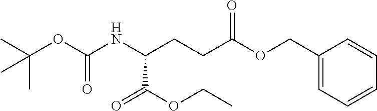 [SMILES: CCOC(=O)[C@@H](CCC(=O)OCc1ccccc1)NC(=O)OC(C)(C)C]

5-benzyl 1-ethyl (tert-butoxycarbonyl)-D-glutamate: To an ice-chilled solution of boc-D-glutamic acid 5-benzyl ester (1.687 g, 5 mmol, 1 eq) in DCM (20 mL) were added DMAP (182 mg, 1.5 mmol, 0.3 eq), absolute ethanol (5 mL) and DCC (1.547 g, 7.5 mmol, 1.5 eq). The resulting mixture was stirred at rt for 19 h. The solvent was evaporated in vacuo and washed twice with diethyl ether to afford the subject compound as a viscous colourless oil (1.827 g, yield: 100%), which was used in the next step without any further purification.

Intermediate 16

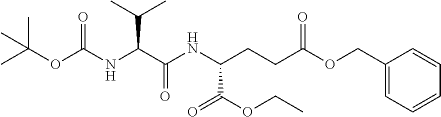 [SMILES: CCOC(=O)[C@@H](CCC(=O)OCc1ccccc1)NC(=O)[C@@H](NC(=O)OC(C)(C)C)C(C)C]

5-benzyl 1-ethyl (tert-butoxycarbonyl)-L-valyl-D-glutamate: Intermediate 15 (1.827 g, 5 mmol, 1 eq) was added to an ice-chilled stirred mixture of TFA and DCM (1/3, 25 mL), and the mixture was allowed to warm to room temperature. After 3 h, the solvent was evaporated in vacuo. The residue was coevaporated three times with diethyl ether. Concurrently, HOBt (0.743 g, 5.5 mmol, 1.1 eq), DCC (1.135 g, 5.5 mmol, 1.1 eq) and DMAP (catalytic amount) were added to an ice-chilled stirring solution of Intermediate 1 (1.194 g, 5.5 mmol, 1.1 eq) in ethyl acetate (25 mL). After 30 minutes, a solution of the deprotected Intermediate 15 in ethyl acetate (15 mL) and TEA (3.5 mL, 25 mmol, 5 eq) was added and the resulting mixture was stirred at rt for 17 h. Hexane (10 mL) was added, after which the mixture was filtered and washed with a 1 M HCl solution (2×40 mL), saturated NaHCO₃ solution (2×40 mL) and brine (40 mL). The organic layer was dried over anhydrous Na₂SO₄ and concentrated in vacuo to afford the subject compound as a viscous yellow oil (2.1 g, yield: 90%). ¹H NMR (400 MHz, DMSO-d₆) δ8.22 (d, 1H), 7.42-7.28 (m, 5H), 6.61 (d, 1H), 5.08 (s, 2H), 4.28-4.18 (m, 1H), 4.13-4.00 (m, 2H), 3.79 (t, 1H), 2.43 (t, 2H), 2.09-1.96 (m, 1H), 1.93-1.81 (m, 1H), 1.79-1.63 (m, 1H), 1.36 (s, 9H), 1.16 (t, 3H), 0.83 (t, 6H).

### Intermediate 17

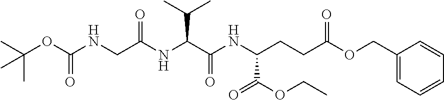 [SMILES: CCOC(=O)[C@@H](CCC(=O)OCc1ccccc1)NC(=O)[C@@H](NC(=O)CNC(=O)OC(C)(C)C)C(C)C]

5-benzyl 1-ethyl (tert-butoxycarbonyl)glycyl-L-valyl-D-glutamate: Intermediate 16 (2.05 g, 4.4 mmol, 1 eq) was added to an ice-chilled stirred mixture of TFA and DCM (1/3, 25 mL), and the mixture was allowed to warm to room temperature. After 3 h, the solvent was evaporated in vacuo. The residue was coevaporated three times with diethyl ether. Concurrently, HOBt (0.653 g, 4.84 mmol, 1.1 eq), DCC (0.999 g, 4.84 mmol, 1.1 eq) and DMAP (catalytic amount) were added to an ice-chilled stirring solution of Intermediate 2 (0.848 g, 4.84 mmol, 1.1 eq) in ethyl acetate (25 mL). After minutes, a solution of the deprotected Intermediate 16 in ethyl acetate (15 mL) and TEA (3.3 mL, 24 mmol, 5 eq) was added and the resulting mixture was stirred at rt for 17 h. Hexane (10 mL) was added, after which the mixture was filtered and washed with a 1 M HCl solution (2×40 mL), saturated NaHCO₃ solution (2×40 mL) and brine (40 mL). The organic layer was dried over anhydrous Na₂SO₄ and concentrated in vacuo to afford the subject compound as a white crystalline solid (1.95 g, yield: 85%). ¹H NMR (400 MHz, DMSO-d₆) 6 8.45 (d, 1H), 7.60 (d, 1H), 7.36 (s, 5H), 7.05 (t, 1H), 5.09 (s, 2H), 4.31— 4.22 (m, 2H), 4.11— 4.03 (m, 2H), 3.58 (d, 2H), 2.45 (t, 2H), 2.09-1.90 (m, 1H), 1.89-1.80 (m, 1H), 1.79-1.62 (m, 1H), 1.38 (s, 9H), 1.17 (t, 3H), 0.82 (t, 6H).

### Intermediate 18

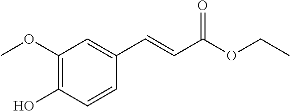 [SMILES: CCOC(=O)/C=C/c1ccc(O)c(OC)c1]

Ethyl (E)-3-(4-hydroxy-3-methoxyphenyl)acrylate: To an ice-chilled suspension of trans-ferulic acid (1.942 g, 10 mmol, 1 eq) in absolute ethanol (20 mL) was added thionyl chloride (0.87 mL, 12 mmol, 1.2 eq). The resulting mixture was refluxed with stirring for 20 h. The solution was concentrated in vacuo, dissolved in ethyl acetate (30 mL) and washed with a 1 M HCl solution (15 mL), saturated NaHCO₃ solution (15 mL) and brine (15 mL). The organic layer was dried over anhydrous Na₂SO₄ and concentrated in vacuo to afford the subject compound as an orange oil (2.185 g, yield: 98%). ¹H NMR (400 MHz, DMSO-d₆) δ9.61 (s, 1H), 7.54 (d, 1H), 7.33 (d, 1H), 7.12 (dd, 1H), 6.79 (d, 1H), 6.48 (d, 1H), 4.16 (q, 2H), 3.81 (s, 3H), 1.25 (t, 3H).

### Intermediate 19

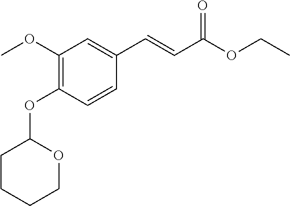 [SMILES: CCOC(=O)/C=C/c1ccc(OC2CCCCO2)c(OC)c1]

Ethyl (E)-3-(3-methoxy-4-((tetrahydro-2H-pyran-2-yl)oxy)phenyl)acrylate: To a solution of Intermediate 18 (2.185 g, 9.8 mmol, 1 eq) in 3,4-dihydropyran (20 mL) was added pyridinium p-toluenesulfonate (198 mg, 0.79 mmol, 0.08 eq). The resulting mixture was refluxed with stirring for 2 h. The solution was concentrated in vacuo, dissolved in ethyl acetate (35 mL) and washed with 1M aqueous NaOH (4×7 mL) and water (2×10 mL). The solvent was evaporated in vacuo to afford the subject compound as a yellow oil (2.8 g, yield: 93%), which was used in the next step without any further purification.

### Intermediate 20

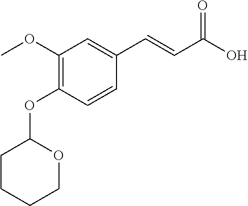 [SMILES: COc1cc(/C=C/C(=O)O)ccc1OC1CCCCO1]

(E)-3-(3-methoxy-4-((tetrahydro-2H-pyran-2-yl)oxy)phenyl)acrylic acid: To a solution of Intermediate 19 (2.8 g, 9.1 mmol) in methanol (20 mL) was added 1 M aqueous NaOH (10 mL). The resulting mixture was stirred at rt for 20 h. The solvent was evaporated in vacuo and the residue was dissolved in water (10 mL) and extracted with ethyl acetate (20 mL). After acidifying the mixture with a 1M HCl solution it was extracted with ethyl acetate (2×30 mL). The combined organic phases were washed with water (2×30 mL) and brine (30 mL), dried over anhydrous Na₂SO₄ and concentrated in vacuo to afford the subject compound as a yellow oil (1.75 g, yield: 69%). ¹H NMR (400 MHz, CDC_{1 3}) δ7.73 (d, 1H), 7.16-7.08 (m, 3H), 6.33 (d, 1H), 5.48 (t, 1H), 3.97-3.86 (m, 4H), 3.67-3.56 (m, 1H), 2.02-1.82 (m, 2H), 1.76-1.58 (m, 4H).

### Intermediate 21

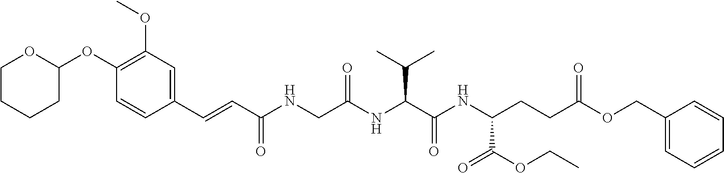 [SMILES: CCOC(=O)[C@@H](CCC(=O)OCc1ccccc1)NC(=O)[C@@H](NC(=O)CNC(=O)/C=C/c1ccc(OC2CCCCO2)c(OC)c1)C(C)C]

5-benzyl 1-ethyl ((E)-3-(3-methoxy-4-((tetrahydro-2H-pyran-2-yl)oxy)phenyl)acryloyl)glycyl-L-valyl-D-glutamate: Intermediate 17 (1.50 g, 2.88 mmol, 1 eq) was added to an ice-chilled stirred mixture of TFA and DCM (1/5, 6 mL), and the mixture was allowed to warm to room temperature. After 4 h, the solvent was evaporated in vacuo. The residue was coevaporated five times with diethyl ether and dissolved in DMF (6 mL). After cooling the solution in ice, DIPEA (2.5 mL, 14.4 mmol, 5 eq), Intermediate 20 (0.80 g, 2.88 mmol, 1 eq), HOBt (0.388 g, 2.88 mmol, 1 eq), DMAP (catalytic amount) and EDC (0.551 g, 2.88 mmol, 1 eq) were added and the resulting mixture was stirred at rt for 18 h. The reaction mixture was diluted with ethyl acetate (100 mL) and washed with a saturated NH₄Cl solution (2×50 mL), saturated NaHCO₃ solution (2×50 mL) and brine (50 mL). The organic layer was dried over anhydrous Na₂SO₄ and concentrated in vacuo to afford the subject compound as an off-white solid (0.753 g, yield: 38%). ¹H NMR (400 MHz, DMSO-d₆) δ8.39 (d, 1H), 8.24 (t, 1H), 7.90 (d, 1H), 7.42-7.27 (m, 6H), 7.21 (d, 1H), 7.09 (d, 2H), 6.65 (d, 1H), 5.45 (t, 1H), 5.06 (s, 2H), 4.33-4.21 (m, 2H), 4.13-4.02 (m, 2H), 3.89 (d, 2H), 3.83-3.75 (m, 4H), 3.58-3.49 (m, 1H), 2.44 (t, 2H), 2.07-1.76 (m, 6H), 1.64-1.52 (m, 3H), 1.17 (t, 3H), 0.84 (t, 6H).

### Intermediate 22

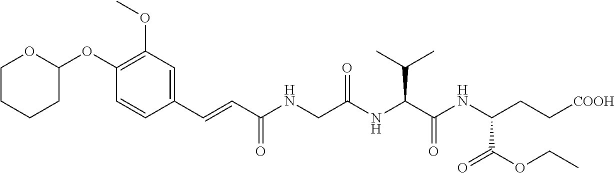 [SMILES: CCOC(=O)[C@@H](CCC(=O)O)NC(=O)[C@@H](NC(=O)CNC(=O)/C=C/c1ccc(OC2CCCCO2)c(OC)c1)C(C)C]

(4R)-5-ethoxy-4-((2S)-2-(2-((E)-3-(3-methoxy-4-((tetrahydro-2H-pyran-2-yl)oxy)phenyl)acrylamido)acetamido)-3-methylbutanamido)-5-oxopentanoic acid: To a solution of palladium (II) acetate (12 mg, 0.055 mmol, 0.05 eq) in dry DCM (7 mL) were added TEA (297 μL, 2.13 mmol, 1.95 eq) and triethylsilane (350 μL, 2.19 mmol, 2 eq). The resulting black solution was stirred for 15 minutes, after which a solution of Intermediate 21 (746 mg, 1.09 mmol, 1 eq) in dry DCM (13 mL) was added. The resulting mixture was stirred under argon atmosphere for 18 h, diluted with DCM (30 mL) and extracted with water (3×30 mL). Combined aqueous phases were acidified with a 1M HCl solution and extracted with DCM (2×50 mL). Combined organic phases were washed with water (2×50 mL) and brine (50 mL), dried over anhydrous Na₂SO₄ and concentrated in vacuo to afford the subject compound as a white solid (355 mg, yield: 55%). ¹H NMR (400 MHz, DMSO-d₆) δ12.17 (s, 1H), 8.49 (s, 1H), 8.36 (s, 1H), 7.88 (d, 1H), 7.37 (d, 1H), 7.22 (s, 1H), 7.10 (d, 2H), 6.68 (d, 1H), 5.45 (t, 1H), 4.30-4.19 (m, 2H), 4.16-4.00 (m, 2H), 3.90 (d, 2H), 3.84-3.74 (m, 4H), 3.58-3.49 (m, 1H), 2.27 (t, 2H), 2.02— 1.75 (m, 6H), 1.64— 1.52 (m, 3H), 1.18 (t, 3H), 0.85 (t, 6H).

### Intermediate 23

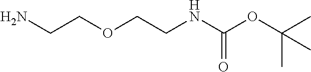

tert-Butyl (2-(2-aminoethoxy)ethyl)carbamate: A solution of Boc₂O (654 mg, 3 mmol, 1 eq) in DCM (15 mL) was added dropwise to a stirring solution of 2-aminoethyl ether dihydrochloride (2.66 g, 15 mmol, 5 eq) in DCM (15 mL) at 0° C. The resulting mixture was stirred at rt for 18 h. Subsequently, the solution was washed with water (3×20 mL) and brine (20 mL), dried over anhydrous Na₂SO₄ and concentrated in vacuo to afford the subject compound as a colourless oil (490 mg, yield: 80%). ¹H NMR (400 MHz, CDCl₃) δ4.95 (brs, 1H), 3.50 (t, 2H), 3.46 (t, 2H), 3.31 (q, 2H), 2.85 (t, 2H), 1.43 (s, 9H), 1.42 (brs, 2H).

### Intermediate 24

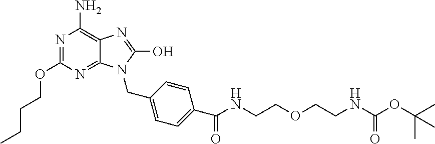 [SMILES: CCCCOc1nc(N)c2nc(O)n(Cc3ccc(C(=O)NCCOCCNC(=O)OC(C)(C)C)cc3)c2n1]

tert-Butyl (2-(2-(4-((6-amino-2-butoxy-8-hydroxy-9H-purin-9-yl)methyl)benzamido)ethoxy)ethyl)carbamate: Compound 10 (200 mg, 0.560 mmol, 1 eq) was dissolved in DMSO (7 mL). Intermediate 23 (380 mg, 1.86 mmol, 3.3 eq) and DIPEA (0.523 mL, 2.99 mmol, 5.3 eq) were dissolved in DCM (1 mL) and added to the stirring solution of Compound 10 in DMSO. After cooling the reaction mixture in ice, COM U (640 mg, 1.49 mmol, 2.7 eq) was added and the mixture was stirred at rt for 3 h. Ethyl acetate (40 mL) and 1 M NaHCO₃ solution (30 mL) were added. After concentrating the mixture in vacuo, the mixture was cooled in ice for one hour. The precipitate was filtered and washed with water and diethyl ether to afford the subject compounds, which was used for the next step without any further purification.

### Intermediate 25

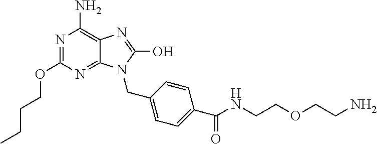 [SMILES: CCCCOc1nc(N)c2nc(O)n(Cc3ccc(C(=O)NCCOCCN)cc3)c2n1]

4-((6-amino-2-butoxy-8-hydroxy-9H-purin-9-yl)methyl)-N-(2-(2-aminoethoxy)ethyl)benzamide: Intermediate 24 (160 mg, 0.294 mmol) was added to an ice-chilled stirred mixture of TFA and DCM (1/5, 6 mL), and the mixture was allowed to warm to room temperature. After 3 h, the solvent was evaporated in vacuo. The residue was precipitated in diethyl ether, filtered and washed with diethyl ether to afford the subject compound as a brown solid (156 mg, yield: 97%). ¹H NMR (400 MHz, DMSO-d₆) δ10.03 (s, 1H), 8.41 (t, 1H), 7.78 (d, 2H), 7.74 (s, 2H), 7.36 (d, 2H), 6.50 (s, 2H), 4.91 (s, 2H), 4.14 (t, 2H), 3.63-3.52 (m, 4H), 3.50-3.41 (m, 2H), 3.03-2.94 (m, 2H), 1.67-1.56 (m, 2H), 1.42-1.30 (m, 2H), 0.90 (t, 3H).

### Compound 26

### SG144

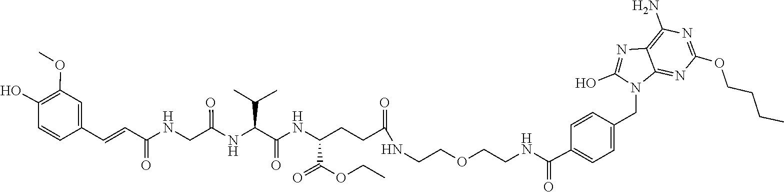 [SMILES: CCCCOc1nc(N)c2nc(O)n(Cc3ccc(C(=O)NCCOCCNC(=O)CC[C@@H](NC(=O)[C@@H](NC(=O)CNC(=O)/C=C/c4ccc(O)c(OC)c4)C(C)C)C(=O)OCC)cc3)c2n1]

Ethyl N5-(2-(2-(4-((6-amino-2-butoxy-8-hydroxy-9H-purin-9-yl)methyl)benzamido)ethoxy)ethyl)-N2-((E)-3-(4-hydroxy-3-methoxyphenyl)acryloyl)glycyl-L- yalyl-D-glutaminate: To an ice-chilled stirring solution of Intermediate 22 (71 mg, 0.120 mmol, 1 eq) in DMF (3 mL) were added DIPEA (84 μL, 0.48 mmol, 4 eq), Intermediate 25 (115 mg, 0.206 mmol, 1.7 eq) and HATU (64 mg, mmol, 1.4 eq). The resulting mixture was stirred at rt for 20 h. Subsequently, 1 M HCl solution (20 mL) was added and the mixture was stirred at rt for 15 minutes, after which it was extracted with a mixture of DCM and isopropanol (3/1, 3×20 mL). The combined organic phases were washed with a saturated NaHCO₃ solution (2×50 mL) and brine (50 mL), dried over anhydrous Na₂SO₄ and concentrated in vacuo to afford the subject compound as an orange solid (48 mg, yield: 39%). ¹H NMR (400 MHz, DMSO-d₆) δ9.99 (s, 1H), 9.43 (s, 1H), 8.44 (t, 1H), 8.38 (d, 1H), 8.19 (t, 1H), 7.89-7.82 (m, 2H), 7.78 (d, 2H), 7.39-7.29 (m, 3H), 7.15 (s, 1H), 7.00 (d, 1H), 6.79 (d, 1H), 6.56 (d, 1H), 6.46 (s, 2H), 4.89 (s, 2H), 4.30-4.22 (m, 2H), 4.22-4.00 (m, 4H), 3.89 (d, 2H), 3.80 (s, 3H), 3.49 (t, 2H), 3.44-3.36 (m, 4H), 3.21-3.15 (m, 2H), 2.14 (t, 2H), 2.02-1.90 (m, 2H), 1.84-1.76 (m, 1H), 1.65-1.55 (m, 2H), 1.41-1.31 (m, 2H), 1.16 (t, 3H), 0.89 (t, 3H), 0.84 (t, 6H). HRMS calculated for C₄₅H₆₁O₁₂N₁₀ m/z: 933.4465 (M+H)⁺, found 933.4458.

### Example 5: Compound RH32

### Intermediate 27

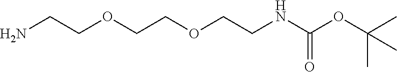 [SMILES: CC(C)(C)OC(=O)NCCOCCOCCN]

tert-Butyl (2-(2-(2-aminoethoxy)ethoxy)ethyl)carbamate: A solution of Boc₂O (4.365 g, 20 mmol, 1 eq) in DCM (40 mL) was added dropwise to a stirring solution of 1,2-Bis(2-aminoethoxy)ethane (14.821 g, 100 mmol, 5 eq) in DCM (100 mL) at 0° C. The resulting mixture was stirred at rt for 20 h. Subsequently, the solution was washed with water (3×100 mL) and brine (100 mL), dried over anhydrous Na₂SO₄ and concentrated in vacuo to afford the subject compound as a white solid (4.370 g, yield: 88%). ¹H NMR (400 MHz, DMSO-d₆) δ6.72 (brs, 1H), 3.53-3.44 (m, 4H), 3.41-3.30 (m, 4H), 3.05 (q, 2H), 2.64 (t, 2H), 1.36 (s, 9H).

### Intermediate 28

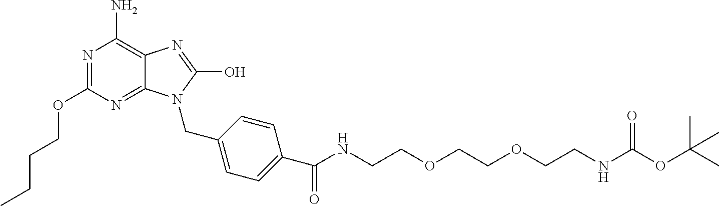 [SMILES: CCCCOc1nc(N)c2nc(O)n(Cc3ccc(C(=O)NCCOCCOCCNC(=O)OC(C)(C)C)cc3)c2n1]

tert-Butyl (2-(2-(2-(4-((6-amino-2-butoxy-8-hydroxy-9H-purin-9-yl)methyl)benzamido)ethoxy)ethoxy)ethyl)carbamate: Compound 10 (500 mg, 1.40 mmol, 1 eq) was dissolved in DMSO (20 mL). Intermediate 27 (1.157 g, 4.66 mmol, 3.3 eq) and DIPEA (1.299 mL, 7.46 mmol, 5.3 eq) were dissolved in DCM (1 mL) and added to the stirring solution of cCompound 10 in DMSO. After cooling the reaction mixture in ice, COMU (1.593 g, 3.72 mmol, 2.7 eq) was added and the mixture was stirred at rt for 19 h. Ethyl acetate (100 mL) and 1 M NaHCO₃ solution (75 mL) were added. After concentrating the mixture in vacuo, the mixture was cooled in ice for one hour. The precipitate was filtered and washed with water and diethyl ether to afford the subject compounds, which was used for the next step without any further purification.

### Intermediate 29

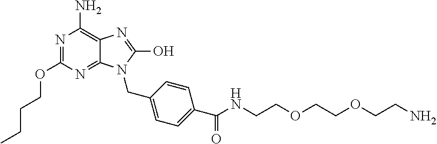 [SMILES: CCCCOc1nc(N)c2nc(O)n(Cc3ccc(C(=O)NCCOCCOCCN)cc3)c2n1]

4-((6-amino-2-butoxy-8-hydroxy-9H-purin-9-yl)methyl)-N-(2-(2-(2-aminoethoxy)ethoxy)ethyl)benzamide: Intermediate 28 (491 mg, 0.84 mmol) was added to an ice-chilled stirred mixture of TFA and DCM (1/5, 12 mL), and the mixture was allowed to warm to room temperature. After 3 h, the solvent was evaporated in vacuo. The residue was precipitated in diethyl ether, filtered and washed with diethyl ether to afford the subject compound as a brown solid (492 mg, yield: 98%). ¹H NMR (400 MHz, DMSO-d₆) δ10.07 (s, 1H), 8.48 (t, 1H), 7.82-7.75 (m, 4H), 7.35 (d, 2H), 6.52 (brs, 2H), 4.91 (s, 2H), 4.13 (t, 2H), 3.63-3.49 (m, 8H), 3.46-3.33 (m, 2H), 3.03-2.88 (m, 2H), 1.67-1.56 (m, 2H), 1.43-1.30 (m, 2H), 0.90 (t, 3H).

### Compound 30

### RH32

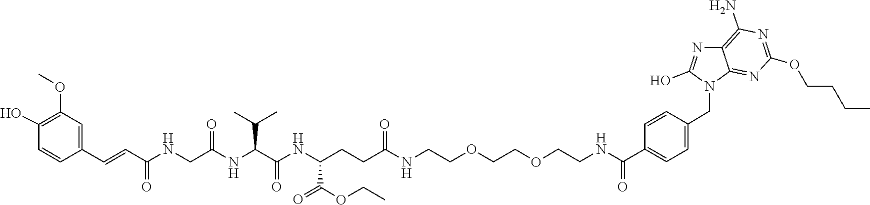 [SMILES: CCCCOc1nc(N)c2nc(O)n(Cc3ccc(C(=O)NCCOCCOCCNC(=O)CC[C@@H](NC(=O)[C@@H](NC(=O)CNC(=O)/C=C/c4ccc(O)c(OC)c4)C(C)C)C(=O)OCC)cc3)c2n1]

Ethyl (R)-1-(44(6-amino-2-butoxy-8-hydroxy-9H-purin-9-yl)methyl)phenyl)-15-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate: To an ice-chilled stirring solution of Intermediate 22 (72 mg, 0.122 mmol, 1 eq) in DMF (4 mL) were added DIPEA (85 μL, 0.49 mmol, 4 eq), Intermediate 29 (132 mg, mmol, 1.8 eq) and COMU (73 mg, 0.17 mmol, 1.4 eq). The resulting mixture was stirred at rt for 20 h. Subsequently, 1 M HCl solution (20 mL) was added and the mixture was stirred at rt for 15 minutes, after which it was extracted with a mixture of DCM and isopropanol (3/1, 3×20 mL). The combined organic phases were washed with a saturated NaHCO₃ solution (2×50 mL) and brine (50 mL), dried over anhydrous Na₂SO₄ and concentrated in vacuo. The crude compound was purified using preparative thin layer silica gel chromatography (10% MeOH/DCM) to afford the subject compound as an orange solid (29 mg, yield: 24%). ¹H NMR (400 MHz, DMSO-d₆) δ9.98 (s, 1H), 9.43 (s, 1H), 8.47 (t, 1H), 8.38 (d, 1H), 8.18 (t, 1H), 7.89-7.82 (m, 2H), 7.78 (d, 2H), 7.38-7.29 (m, 3H), 7.15 (s, 1H), 7.00 (d, 1H), 6.79 (d, 1H), 6.57 (d, 1H), 6.46 (s, 2H), 4.90 (s, 2H), 4.30-4.01 (m, 6H), 3.89 (d, 2H), 3.80 (s, 3H), 3.56-3.44 (m, 10H), 3.19-3.13 (m, 2H), 2.13 (t, 2H), 2.02-1.88 (m, 2H), 1.86-1.75 (m, 1H), 1.65-1.57 (m, 2H), 1.41-1.31 (m, 2H), 1.17 (t, 3H), 0.93-0.80 (m, 9H). HRMS calculated for C₄₇H63013N10 m/z: 975.4582 (M-H)-, found 975.4589.

### Example 6: Compound SG176

### Intermediate 31

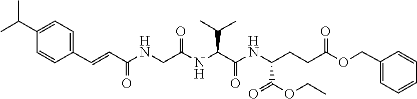 [SMILES: CCOC(=O)[C@@H](CCC(=O)OCc1ccccc1)NC(=O)[C@@H](NC(=O)CNC(=O)/C=C/c1ccc(C(C)C)cc1)C(C)C]

5-benzyl 1-ethyl ((E)-3-(4-isopropyl phenyl)acryloyl)glycyl-L-valyl-D-glutamate: Intermediate 17 (0.500 g, 0.96 mmol, 1 eq) was added to an ice-chilled stirred mixture of TFA and DCM (1/4, 5 mL), and the mixture was allowed to warm to room temperature. After 3 h, the solvent was evaporated in vacuo. The residue was coevaporated four times with diethyl ether and dissolved in DMF (5 mL). After cooling the solution in ice, DIPEA (0.84 mL, 4.79 mmol, 5 eq), (E)-3-(4-isopropylphenyl)acrylic acid (0.22 g, 1.15 mmol, 1.2 eq), HOBt (0.155 g, 1.15 mmol, 1.2 eq), DMAP (catalytic amount) and EDC (0.220 g, 1.15 mmol, 1.2 eq) were added and the resulting mixture was stirred at rt for 18 h. The reaction mixture was diluted with ethyl acetate (60 mL) and washed with a 1 M HCl solution (2×30 mL), saturated NaHCO₃ solution (2×30 mL) and brine (30 mL). The organic layer was dried over anhydrous Na₂SO₄ and concentrated in vacuo to afford the subject compound as an off-white solid (0.392 g, yield: 69%). ¹H NMR (400 MHz, DMSO-d₆) δ8.39 (d, 1H), 8.35 (t, 1H), 7.94 (d, 1H), 7.49 (d, 2H), 7.44-7.25 (m, 8H), 6.69 (d, 1H), 5.06 (s, 2H), 4.32-4.19 (m, 2H), 4.13-4.00 (m, 2H), 3.90 (d, 2H), 2.90 (p, 1H), 2.43 (t, 2H), 2.08-1.93 (m, 2H), 1.93-1.80 (m, 1H), 1.23-1.14 (m, 9H), 0.84 (t, 6H).

### Intermediate 32

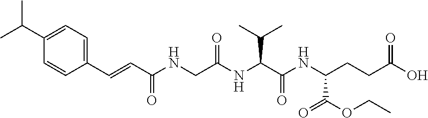 [SMILES: CCOC(=O)[C@@H](CCC(=O)O)NC(=O)[C@@H](NC(=O)CNC(=O)/C=C/c1ccc(C(C)C)cc1)C(C)C]

(R)-5-ethoxy-4-((S)-2-(2-((E)-3-(4-isopropylphenyl)acrylamido)acetamido)-3-methylbutanamido)-5-oxopentanoic acid: To a solution of palladium (II) acetate (7 mg, 0.031 mmol, 0.05 eq) in dry DCM (5 mL) were added TEA (167 μL, 1.20 mmol, 1.95 eq) and triethylsilane (197 μL, 1.23 mmol, 2 eq). The resulting black solution was stirred for 15 minutes, after which a solution of Intermediate 31 (366 mg, 0.616 mmol, 1 eq) in dry DCM (10 mL) was added. The resulting mixture was stirred under argon atmosphere for 18 h, diluted with DCM (25 mL) and extracted with water (4×30 mL). Combined aqueous phases were acidified with a 1M HCl solution and extracted with DCM (2×30 mL). Combined organic phases were washed with water (30 mL) and brine (30 mL), dried over anhydrous Na₂SO₄ and concentrated in vacuo to afford the subject compound as a white solid (133 mg, yield: 43%). ¹H NMR (400 MHz, DMSO-d₆) δ12.18 (s, 1H), 8.43-8.31 (m, 2H), 7.93 (d, 1H), 7.49 (d, 2H), 7.40 (d, 1H), 7.29 (d, 2H), 6.69 (d, 1H), 4.31-4.21 (m, 2H), 4.14-4.01 (m, 2H), 3.90 (d, 2H), 2.95-2.85 (m, 1H), 2.28 (t, 2H), 2.03-1.91 (m, 2H), 1.88-1.74 (m, 1H), 1.23-1.13 (m, 9H), 0.87-0.81 (m, 6H).

### Compound 33

### SG176

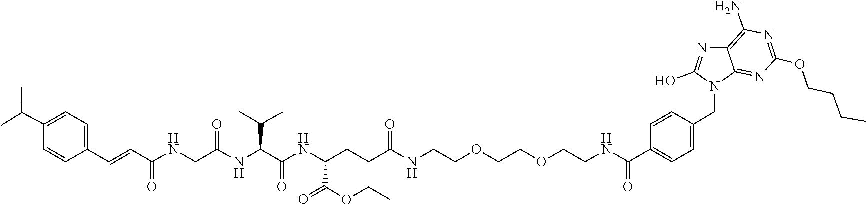 [SMILES: CCCCOc1nc(N)c2nc(O)n(Cc3ccc(C(=O)NCCOCCOCCNC(=O)CC[C@@H](NC(=O)[C@@H](NC(=O)CNC(=O)/C=C/c4ccc(C(C)C)cc4)C(C)C)C(=O)OCC)cc3)c2n1]

Ethyl (R)-1-(4-((6-amino-2-butoxy-8-hydroxy-9H-purin-9-yl)methyl)phenyl)-15-((S)-2-(2-((E)-3-(4-isopropylphenyl)acrylamido)acetamido)-3- methylbutanamido)-1,12-dioxo-5,8-dioxa-2,11-diazahexadecan-16-oate: To an ice-chilled stirring solution of Intermediate 32 (130 mg, 0.26 mmol, 1 eq) in DMF (3 mL) were added DIPEA (135 μL, 0.774 mmol, 3 eq), Intermediate 29 (171 mg, 0.28 mmol, 1.1 eq) and COMU (133 mg, 0.31 mmol, 1.2 eq). The resulting mixture was stirred at rt for 20 h. Subsequently, 1 M HCl solution (40 mL) was added, after which the mixture was extracted with a mixture of DCM and isopropanol (3/1, 3×30 mL). The combined organic phases were washed with a saturated NaHCO₃ solution (2×40 mL) and brine (40 mL), dried over anhydrous Na₂SO₄ and concentrated in vacuo. The solid residue was washed with diethyl ether to afford the subject compound as an off-white solid (73 mg, yield: 29%). ¹H NMR (400 MHz, DMSO-d₆) δ10.03 (s, 1H), 8.48 (t, 1H), 8.41-8.30 (m, 2H), 7.91 (d, 1H), 7.90-7.85 (m, 1H), 7.79 (d, 2H), 7.49 (d, 2H), 7.40 (d, 1H), 7.34 (d, 2H), 7.31-7.25 (m, 2H), 6.70 (d, 1H), 6.49 (s, 2H), 4.90 (s, 2H), 4.32-4.02 (m, 6H), 3.92 (d, 2H), 3.56-3.45 (m, 6H), 3.41-3.35 (m, 4H), 3.21-3.12 (m, 2H), 2.93-2.85 (m, 1H), 2.14 (t, 2H), 2.05-1.89 (m, 2H), 1.87-1.73 (m, 1H), 1.66-1.56 (m, 2H), 1.41-1.31 (m, 2H), 1.22-1.13 (m, 9H), 0.92-0.79 (m, 9H). HRMS calculated for C₄₉H₆₉O₁₁N₁₀ m/z: 973.5142 (M+H)⁺, found 973.5112.

### Example 7: Compound SG177

### Intermediate 34

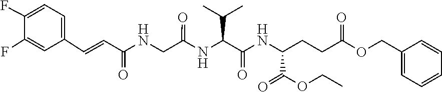 [SMILES: CCOC(=O)[C@@H](CCC(=O)OCc1ccccc1)NC(=O)[C@@H](NC(=O)CNC(=O)/C=C/c1ccc(F)c(F)c1)C(C)C]

5-benzyl 1-ethyl ((E)-3-(3,4-difluorophenyl)acryloyl)glycyl-L-valyl-D-glutamate: Intermediate 17 (0.500 g, 0.96 mmol, 1 eq) was added to an ice-chilled stirred mixture of TFA and DCM (1/4, 5 mL), and the mixture was allowed to warm to room temperature. After 3 h, the solvent was evaporated in vacuo. The residue was coevaporated four times with diethyl ether and dissolved in DMF (5 mL). After cooling the solution in ice, DIPEA (0.84 mL, 4.79 mmol, 5 eq), (E)-3-(3,4-difluorophenyl)acrylic acid (0.21 g, 1.15 mmol, 1.2 eq), HOBt (0.155 g, 1.15 mmol, 1.2 eq), DMAP (catalytic amount) and EDC (0.220 g, 1.15 mmol, 1.2 eq) were added and the resulting mixture was stirred at rt for 18 h. The reaction mixture was diluted with ethyl acetate (60 mL) and washed with a 1 M HCl solution (2×30 mL), saturated NaHCO₃ solution (2×30 mL) and brine (30 mL). The organic layer was dried over anhydrous Na₂SO₄ and concentrated in vacuo to afford the subject compound as an off-white solid (0.364 g, yield: 65%). ¹H NMR (400 MHz, DMSO-d₆) δ8.41 (d, 1H), 8.35 (t, 1H), 7.96 (d, 1H), 7.74-7.63 (m, 1H), 7.53-7.27 (m, 8H), 6.74 (d, 1H), 5.07 (s, 2H), 4.33-4.20 (m, 2H), 4.13-4.03 (m, 2H), 3.91 (d, 2H), 2.44 (t, 2H), 2.10-1.92 (m, 2H), 1.91— 1.79 (m, 1H), 1.17 (t, 3H), 0.84 (t, 6H).

### Intermediate 35

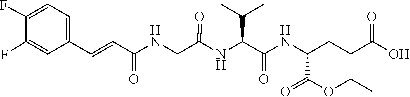 [SMILES: CCOC(=O)[C@@H](CCC(=O)O)NC(=O)[C@@H](NC(=O)CNC(=O)/C=C/c1ccc(F)c(F)c1)C(C)C]

(R)-4-((S)-2-(2-((E)-3-(3,4-difluorophenyl)acrylamido)acetamido)-3-methylbutanamido)-5-ethoxy-5-oxopentanoic acid: To a solution of palladium (II) acetate (6 mg, 0.027 mmol, 0.05 eq) in dry DCM (5 mL) were added TEA (145 μL, 1.04 mmol, 1.95 eq) and triethylsilane (171 μL, 1.07 mmol, 2 eq). The resulting black solution was stirred for 15 minutes, after which a solution of Intermediate 34 (314 mg, 0.534 mmol, 1 eq) in dry DCM (10 mL) was added. The resulting mixture was stirred under argon atmosphere for 18 h, diluted with DCM (25 mL) and extracted with water (4×30 mL). Combined aqueous phases were acidified with a 1M HCl solution and extracted with DCM (2×30 mL). Combined organic phases were washed with water (30 mL) and brine (30 mL), dried over anhydrous Na₂SO₄ and concentrated in vacuo to afford the subject compound as a white solid (64 mg, yield: 24%). ¹H NMR (400 MHz, DMSO-d₆) δ12.18 (s, 1H), 8.43-8.32 (m, 2H), 7.95 (d, 1H), 7.75-7.64 (m, 1H), 7.52-7.38 (m, 2H), 7.36-7.24 (m, 1H), 6.74 (d, 1H), 4.30-4.21 (m, 2H), 4.13-4.02 (m, 2H), 3.91 (d, 2H), 2.28 (t, 2H), 2.02-1.89 (m, 2H), 1.85-1.73 (m, 1H), 1.17 (t, 3H), 0.87-0.79 (m, 6H).

### Compound 36

### SG177

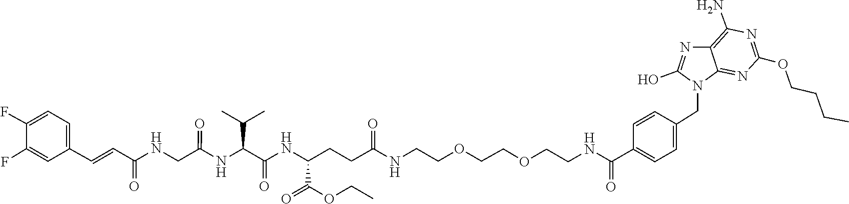 [SMILES: CCCCOc1nc(N)c2nc(O)n(Cc3ccc(C(=O)NCCOCCOCCNC(=O)CC[C@@H](NC(=O)[C@@H](NC(=O)CNC(=O)/C=C/c4ccc(F)c(F)c4)C(C)C)C(=O)OCC)cc3)c2n1]

Ethyl (R)-1-(4-((6-amino-2-butoxy-8-hydroxy-9H-purin-9-yl)methyl)phenyl)-15-((S)-2-(2-((E)-3-(3,4-difluorophenyl)acrylamido)acetamido)-3- methylbutanamido)-1,12-dioxo-5,8-dioxa-2,11-diazahexadecan-16-oate: To an ice-chilled stirring solution of Intermediate 35 (64 mg, 0.13 mmol, 1 eq) in DMF (3 mL) were added DIPEA (67 μL, 0.39 mmol, 3 eq), Intermediate 29 (93 mg, 0.15 mmol, 1.2 eq) and COMU (66 mg, 0.15 mmol, 1.2 eq). The resulting mixture was stirred at rt for 20 h. Subsequently, 1 M HCl solution (40 mL) was added, after which the mixture was extracted with a mixture of DCM and isopropanol (3/1, 3×15 mL). The combined organic phases were washed with a saturated NaHCO₃ solution (3×40 mL) and brine (40 mL), dried over anhydrous Na₂SO₄ and concentrated in vacuo. The solid residue was washed with diethyl ether to afford the subject compound as an off-white solid (42 mg, yield: 34%). ¹H NMR (400 MHz, DMSO-d₆) δ9.98 (s, 1H), 8.48 (t, 1H), 8.42-8.31 (m, 2H), 7.94 (d, 1H), 7.87 (t, 1H), 7.78 (d, 2H), 7.73-7.62 (m, 1H), 7.53-7.22 (m, 6.74 (d, 1H), 6.47 (s, 2H), 4.90 (s, 2H), 4.34-4.02 (m, 6H), 3.92 (d, 2H), 3.58-3.45 (m, 6H), 3.42-3.35 (m, 4H), 3.22-3.09 (m, 2H), 2.14 (t, 2H), 2.03-1.89 (m, 2H), 1.87-1.73 (m, 1H), 1.67-1.55 (m, 2H), 1.41-1.31 (m, 2H), 1.21-1.13 (m, 3H), 0.94-0.76 (m, 9H). HRMS calculated for C₄₆H₆₁O₁₁N₁₀F₂ m/z: 967.4484 (M+H)⁺, found 967.4455.

### Example 8: Compound SG187

### Intermediate 37

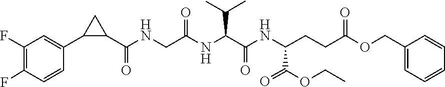 [SMILES: CCOC(=O)[C@@H](CCC(=O)OCc1ccccc1)NC(=O)[C@@H](NC(=O)CNC(=O)C1CC1c1ccc(F)c(F)c1)C(C)C]

1-ethyl (2-(3,4-difluorophenyl)cyclopropane-1-carbonyl)glycyl-L-yalyl-D-glutamate: Intermediate 17 (0.500 g, 0.96 mmol, 1 eq) was added to an ice-chilled stirred mixture of TFA and DCM (1/4, 4 mL), and the mixture was allowed to warm to room temperature. After 3 h, the solvent was evaporated in vacuo. The residue was coevaporated four times with diethyl ether and dissolved in DMF (5 mL). After cooling the solution in ice, DIPEA (0.84 mL, 4.79 mmol, 5 eq), 2-(3,4-difluorophenyl)cyclopropane-1-carboxylic acid (0.209 g, 1.05 mmol, 1.1 eq), HOBt (0.142 g, 1.05 mmol, 1.1 eq), DMAP (catalytic amount) and EDC (0.202 g, 1.05 mmol, 1.1 eq) were added and the resulting mixture was stirred at rt for 18 h. The reaction mixture was diluted with ethyl acetate (60 mL) and washed with a 1 M HCl solution (2×30 mL), saturated NaHCO₃ solution (2×30 mL) and brine (30 mL). The organic layer was dried over anhydrous Na₂SO₄ and concentrated in vacuo to afford the subject compound as a white solid (0.478 g, yield: 81%). ¹H NMR (400 MHz, DMSO-d₆) δ8.45-8.36 (m, 2H), 7.84 (dd, 1H), 7.42-7.27 (m, 6H), 7.27-7.17 (m, 1H), 7.05-6.96 (m, 1H), 5.08 (d, 2H), 4.31-4.20 (m, 2H), 4.14-3.99 (m, 2H), 3.81 (d, 2H), 2.44 (t, 2H), 2.31-2.22 (m, 1H), 2.07-1.91 (m, 3H), 1.90-1.78 (m, 1H), 1.37-1.30 (m, 1H), 1.29-1.20 (m, 1H), 1.16 (t, 3H), 0.82 (t, 6H).

### Intermediate 38

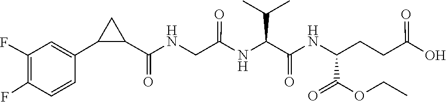 [SMILES: CCOC(=O)[C@@H](CCC(=O)O)NC(=O)[C@@H](NC(=O)CNC(=O)C1CC1c1ccc(F)c(F)c1)C(C)C]

(4R)-4-((2S)-2-(2-(2-(3,4-difluorophenyl)cyclopropane-1-carboxamido)acetamido)-3-methylbutanamido)-5-ethoxy-5-oxopentanoic acid: To a solution of palladium (II) acetate (34 mg, 0.15 mmol, 0.2 eq) in dry DCM (5 mL) were added TEA (208 μL, 1.50 mmol, 2 eq) and a solution of Intermediate 37 (461 mg, 0.75 mmol, 1 eq) in dry DCM (10 mL). After stirring for 5 minutes, triethylsilane (359 μL, 2.25 mmol, 3 eq) was added dropwise and the resulting mixture was stirred under argon atmosphere for 18 h. Following quenching by the addition of water (30 mL), the mixture was filtered, acidified with a 1M HCL solution and extracted with DCM (3×30 mL). Combined organic phases were washed with water (50 mL) and brine (50 mL), dried over anhydrous Na₂SO₄ and concentrated in vacuo to afford the subject compound as a red solid (214 mg, yield: 56%). ¹H NMR (400 MHz, DMSO-d₆) δ12.19 (s, 1H), 8.53-8.34 (m, 2H), 7.83 (dd, 1H), 7.37-7.28 (m, 1H), 7.28-7.19 (m, 1H), 7.07-6.95 (m, 1H), 4.29-4.19 (m, 2H), 4.14-4.01 (m, 2H), 3.82 (d, 2H), 2.31-2.24 (m, 3H), 2.04-1.90 (m, 3H), 1.85-1.73 (m, 1H), 1.37-1.30 (m, 1H), 1.29-1.23 (m, 1H), 1.18 (t, 3H), 0.84 (t, 6H).

### Compound 39

### SG187

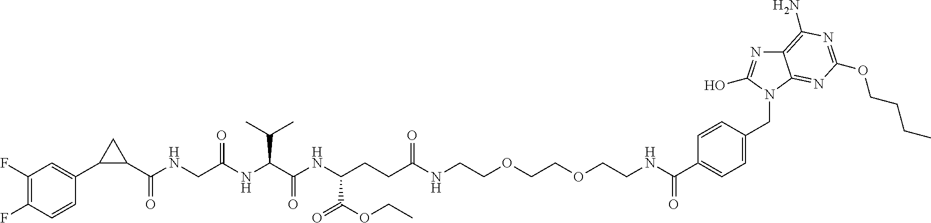 [SMILES: CCCCOc1nc(N)c2nc(O)n(Cc3ccc(C(=O)NCCOCCOCCNC(=O)CC[C@@H](NC(=O)[C@@H](NC(=O)CNC(=O)C4CC4c4ccc(F)c(F)c4)C(C)C)C(=O)OCC)cc3)c2n1]

Ethyl (15R)-1-(4-((6-amino-2-butoxy-8-hydroxy-9H-purin-9-yl)methyl)phenyl)-15-((2S)-2-(2-(2-(3,4-difluorophenyl)cyclopropane-1- carboxamido)acetamido)-3-methylbutanamido)-1,12-dioxo-5,8-dioxa-2,11-diazahexadecan-16-oate: To an ice-chilled stirring solution of Intermediate 38 (70 mg, 0.14 mmol, 1 eq) in DMF (3 mL) were added DIPEA (71 μL, 0.41 mmol, 3 eq), Intermediate 29 (82 mg, mmol, 1 eq) and COM U (64 mg, 0.15 mmol, 1.1 eq). The resulting mixture was stirred at rt for 20 h. Subsequently, 1 M HCl solution (40 mL) was added, after which the mixture was extracted with a mixture of DCM and isopropanol (3/1, 2×40 mL). The combined organic phases were washed with a saturated NaHCO₃ solution (3×40 mL) and brine (40 mL), dried over anhydrous Na₂SO₄ and concentrated in vacuo. The solid residue was washed with diethyl ether to afford the subject compound as an off-white solid (62 mg, yield: 47%). ¹H NMR (400 MHz, DMSO-d₆) δ10.00 (s, 1H), 8.49 (q, 1H), 8.40 (t, 2H), 7.92-7.74 (m, 4H), 7.43-7.27 (m, 3H), 7.27-7.17 (m, 1H), 7.05-6.95 (m, 1H), 6.48 (s, 2H), 4.90 (s, 2H), 4.30-4.00 (m, 6H), 3.81 (d, 2H), 3.55-3.45 (m, 6H), 3.44-3.35 (m, 4H), 3.21-3.12 (m, 2H), 2.31-2.22 (m, 1H), 2.13 (t, 2H), 2.04-1.87 (m, 3H), 1.86-1.72 (m, 1H), 1.66-1.55 (m, 2H), 1.41-1.29 (m, 3H), 1.29-1.21 (m, 1H), 1.16 (t, 3H), 0.89 (t, 3H), 0.83 (t, 6H). HRMS calculated for C₄₇H63011N1oF2 m/z: 981.4640 (M+H)+, found 981.4605.

### Example 9: Compound SG188

### Intermediate 40

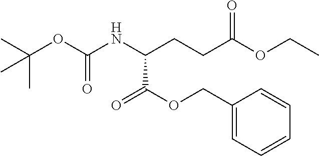 [SMILES: CCOC(=O)CC[C@@H](NC(=O)OC(C)(C)C)C(=O)OCc1ccccc1]

1-benzyl 5-ethyl (tert-butoxycarbonyl)-D-glutamate: To a solution of boc-D-glutamic acid 1-benzyl ester (3.374 g, 10 mmol, 1 eq) in DCM (50 mL) were added DMAP (0.122 g, 1 mmol, 0.1 eq), absolute ethanol (5 mL), HOBt (2.027 g, 15 mmol, 1.5 eq) and EDC (2.876 g, 15 mmol, 1.5 eq). The resulting mixture was stirred at rt for 18 h. The reaction mixture was diluted with DCM (50 mL) and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as a yellow oil (3.605 g, yield: 99%). ¹H NMR (400 MHz, DMSO-d₆) δ7.42-7.29 (m, 6H), (q, 2H), 4.10-3.99 (m, 3H), 2.40-2.32 (m, 2H), 2.01-1.91 (m, 1H), 1.88-1.74 (m, 1H), 1.37 (s, 9H), 1.16 (t, 3H).

### Intermediate 41

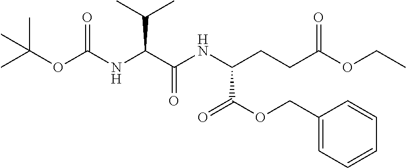 [SMILES: CCOC(=O)CC[C@@H](NC(=O)[C@@H](NC(=O)OC(C)(C)C)C(C)C)C(=O)OCc1ccccc1]

1-benzyl 5-ethyl (tert-butoxycarbonyl)-L-valyl-D-glutamate: Intermediate 40 (3.515 g, 9.62 mmol, 1 eq) was added to an ice-chilled stirred mixture of TFA and DCM (1/4, 25 mL), and the mixture was allowed to warm to room temperature. After 3 h, the solvent was evaporated in vacuo. The residue was coevaporated four times with diethyl ether and dissolved in DCM (30 mL). After cooling the solution in ice, DIPEA (8.38 mL, 48.1 mmol, 5 eq), Intermediate 1 (2.507 g, 11.54 mmol, 1.2 eq), HOBt (1.559 g, 11.54 mmol, 1.2 eq), DMAP (catalytic amount) and EDC (2.212 g, 11.54 mmol, 1.2 eq) were added and the resulting mixture was stirred at rt for 18 h. The reaction mixture was diluted with DCM (30 mL) and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as a yellow oil (4.370 g, yield: 98%). ¹H NMR (400 MHz, DMSO-d₆) δ8.30 (d, 1H), 7.42-7.31 (m, 5H), 6.64 (d, 1H), 5.12 (d, 2H), 4.37-4.26 (m, 1H), 4.03 (q, 2H), 3.86-3.77 (m, 1H), 2.36 (t, 2H), 2.09-1.97 (m, 1H), 1.94-1.79 (m, 2H), 1.38 (s, 9H), 1.16 (t, 3H), 0.86-0.78 (m, 6H).

### Intermediate 42

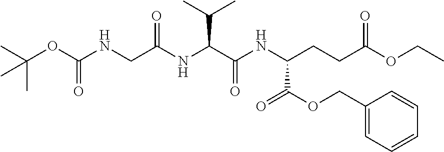 [SMILES: CCOC(=O)CC[C@@H](NC(=O)[C@@H](NC(=O)CNC(=O)OC(C)(C)C)C(C)C)C(=O)OCc1ccccc1]

1-benzyl 5-ethyl (tert-butoxycarbonyl)glycyl-L-valyl-D-glutamate: Intermediate 41 (4.238 g, 9.12 mmol, 1 eq) was added to an ice-chilled stirred mixture of TFA and DCM (1/4, 25 mL), and the mixture was allowed to warm to room temperature. After 3 h, the solvent was evaporated in vacuo. The residue was coevaporated four times with diethyl ether and dissolved in DCM (30 mL). After cooling the solution in ice, DIPEA (7.94 mL, 45.6 mmol, 5 eq), Intermediate 2 (1.918 g, 10.95 mmol, 1.2 eq), HOBt (1.479 g, 10.95 mmol, 1.2 eq), DMAP (catalytic amount) and EDC (2.099 g, 10.95 mmol, 1.2 eq) were added and the resulting mixture was stirred at rt for 18 h. The reaction mixture was diluted with DCM (30 mL) and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as an off-white solid (3.672 g, yield: 77%). ¹H NMR (400 MHz, DMSO-d₆) δ8.49 (d, 1H), 7.62 (d, 1H), 7.43-7.27 (m, 5H), 7.04 (t, 1H), 5.12 (d, 2H), 4.40-4.21 (m, 2H), 4.03 (q, 2H), 3.58 (d, 2H), 2.35 (t, 2H), 2.08-1.97 (m, 1H), 1.97-1.88 (m, 1H), 1.88-1.76 (m, 1H), 1.38 (s, 9H), 1.15 (t, 3H), 0.89-0.72 (m, 6H).

### Intermediate 43

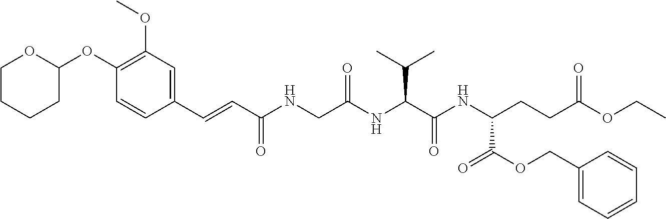 [SMILES: CCOC(=O)CC[C@@H](NC(=O)[C@@H](NC(=O)CNC(=O)/C=C/c1ccc(OC2CCCCO2)c(OC)c1)C(C)C)C(=O)OCc1ccccc1]

1-benzyl 5-ethyl ((E)-3-(3-methoxy-4-((tetrahydro-2H-pyran-2-yl)oxy)phenyl)acryloyl)glycyl-L-valyl-D-glutamate: Intermediate 42 (500 mg, 0.96 mmol, 1 eq) was added to an ice-chilled stirred mixture of TFA and DCM (1/5, 5 mL), and the mixture was allowed to warm to room temperature. After 2 h, the solvent was evaporated in vacuo. The residue was coevaporated four times with diethyl ether and dissolved in DMF (5 mL). After cooling the solution in ice, DIPEA (0.835 mL, 4.80 mmol, 5 eq), Intermediate 20 (294 mg, 1.06 mmol, 1.1 eq), HOBt (143 mg, 1.06 mmol, 1.1 eq), DMAP (catalytic amount) and EDC (202 mg, 1.06 mmol, 1.1 eq) were added and the resulting mixture was stirred at rt for 18 h. The reaction mixture was diluted with ethyl acetate (60 mL) and washed with a saturated NH₄Cl solution (2×30 mL), saturated NaHCO₃ solution (2×30 mL) and brine (30 mL). The organic layer was dried over anhydrous Na₂SO₄ and concentrated in vacuo. The crude compound was purified using silica gel chromatography (4% MeOH/DCM) to afford the subject compound as a white solid (251 mg, yield: 38%). ¹H NMR (400 MHz, DMSO-d₆) δ8.45 (d, 1H), 8.25 (t, 1H), 7.94 (d, 1H), 7.41-7.29 (m, 6H), 7.21 (s, 1H), 7.10 (s, 2H), 6.66 (d, 1H), 5.46 (t, 1H), 5.13 (d, 2H), 4.39-4.31 (m, 1H), 4.30-4.23 (m, 1H), 4.01 (q, 2H), 3.90 (d, 2H), 3.86-3.76 (m, 4H), 3.59-3.49 (m, 1H), 2.36 (t, 2H), 2.09-1.93 (m, 2H), 1.92-1.73 (m, 4H), 1.67-1.48 (m, 3H), 1.14 (t, 3H), 0.83 (t, 6H).

### Intermediate 44

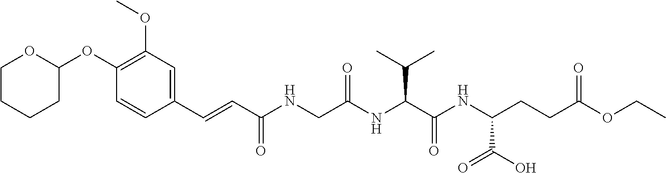 [SMILES: CCOC(=O)CC[C@@H](NC(=O)[C@@H](NC(=O)CNC(=O)/C=C/c1ccc(OC2CCCCO2)c(OC)c1)C(C)C)C(=O)O]

(2R)-5-ethoxy-2-((2S)-2-(2-((E)-3-(3-methoxy-4-((tetrahydro-2H-pyran-2-yl)oxy)phenyl)acrylamido)acetamido)-3-methylbutanamido)-5-oxopentanoic acid: To a solution of palladium (II) acetate (4 mg, 0.016 mmol, 0.05 eq) in dry DCM (5 mL) were added TEA (87 μL, mmol, 1.95 eq) and triethylsilane (102 μL, 0.64 mmol, 2 eq). The resulting black solution was stirred for 15 minutes, after which a solution of Intermediate 43 (218 mg, 0.32 mmol, 1 eq) in dry DCM (7 mL) was added. The resulting mixture was stirred under argon atmosphere for 18 h, diluted with DCM (20 mL) and extracted with water (3×30 mL). Combined aqueous phases were acidified with a 1M HCl solution and extracted with DCM (3×30 mL). Combined organic phases were washed with water (50 mL) and brine (50 mL), dried over anhydrous Na₂SO₄ and concentrated in vacuo to afford the subject compound as a white solid (141 mg, yield: 75%). ¹H NMR (400 MHz, DMSO-d₆) δ12.68 (s, 1H), 8.29 (d, 1H), 8.24 (t, 1H), 7.90 (d, 1H), 7.37 (d, 1H), 7.21 (s, 1H), 7.10 (s, 2H), 6.65 (d, 1H), 5.46 (t, 1H), 4.30-4.19 (m, 2H), 4.02 (q, 2H), 3.89 (d, 2H), 3.82 (s, 3H), 3.80-3.74 (m, 1H), 3.57-3.49 (m, 1H), 2.33 (t, 2H), 2.06-1.93 (m, 2H), 1.92-1.71 (m, 4H), 1.69-1.48 (m, 3H), 1.15 (t, 3H), (t, 6H).

### Compound 45

### SG188

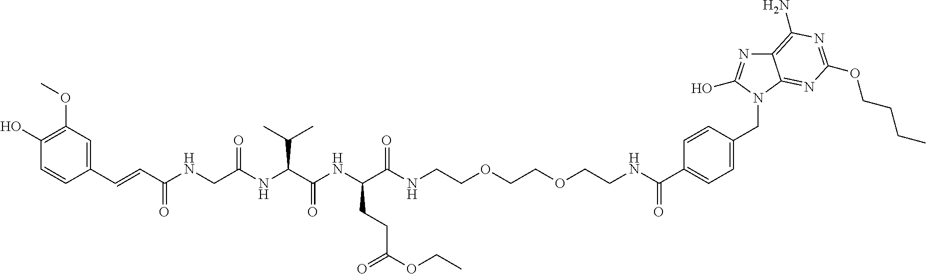 [SMILES: CCCCOc1nc(N)c2nc(O)n(Cc3ccc(C(=O)NCCOCCOCCNC(=O)[C@@H](CCC(=O)OCC)NC(=O)[C@@H](NC(=O)CNC(=O)/C=C/c4ccc(O)c(OC)c4)C(C)C)cc3)c2n1]

Ethyl (R)-1-(4-((6-amino-2-butoxy-8-hydroxy-9H-purin-9-yl)methyl)phenyl)-13-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate: To an ice-chilled stirring solution of Intermediate 44 (80 mg, 0.14 mmol, 1 eq) in DMF (3 mL) were added DIPEA (71 μL, 0.41 mmol, 3 eq), Intermediate 29 (82 mg, 0.14 mmol, 1 eq) and COMU (64 mg, 0.15 mmol, 1.1 eq). The resulting mixture was stirred at rt for 20 h. Subsequently, 1 M HCl solution (40 mL) was added and the mixture was stirred at rt for 15 minutes, after which it was extracted with a mixture of DCM and isopropanol (3/1, 3×30 mL). The combined organic phases were washed with a saturated NaHCO₃ solution (3×40 mL) and brine (40 mL), dried over anhydrous Na₂SO₄ and concentrated in vacuo. The crude compound was purified using silica gel chromatography (10% MeOH/DCM) to afford the subject compound as a white solid (72 mg, yield: 54%). ¹H NMR (400 MHz, DMSO-d₆) δ9.98 (s, 1H), 9.44 (s, 1H), 8.48 (t, 1H), 8.28 (d, 1H), 8.20-8.12 (m, 1H), 7.97 (d, 1H), 7.91 (t, 1H), 7.78 (d, 2H), 7.37-7.28 (m, 3H), 7.14 (d, 1H), 7.00 (dd, 1H), 6.79 (d, 1H), 6.55 (d, 1H), 6.47 (s, 2H), 4.90 (s, 2H), 4.27-4.10 (m, 4H), 4.01 (q, 2H), 3.88 (d, 2H), 3.80 (s, 3H), 3.53-3.44 (m, 6H), 3.42-3.35 (m, 4H), 3.27-3.08 (m, 2H), 2.31-2.23 (m, 2H), 2.00-1.87 (m, 2H), 1.81— 1.68 (m, 1H), 1.65-1.55 (m, 2H), 1.42-1.30 (m, 2H), 1.14 (t, 3H), 0.93-0.81 (m, 9H). HRMS calculated for C₄₇H₆₅O₁₃N₁₀ m/z: 977.4727 (M+H)⁺, found 977.4691.

### Example 10: Compound SG189

### Intermediate 46

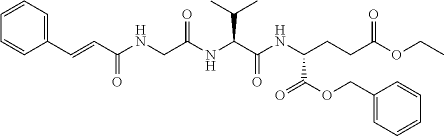 [SMILES: CCOC(=O)CC[C@@H](NC(=O)[C@@H](NC(=O)CNC(=O)/C=C/c1ccccc1)C(C)C)C(=O)OCc1ccccc1]

1-benzyl 5-ethyl cinnamoylglycyl-L-valyl-D-glutamate: Intermediate 42 (500 mg, 0.96 mmol, 1 eq) was added to an ice-chilled stirred mixture of TFA and DCM (1/5, 5 mL), and the mixture was allowed to warm to room temperature. After 2 h, the solvent was evaporated in vacuo. The residue was coevaporated four times with diethyl ether and dissolved in DMF (5 mL). After cooling the solution in ice, DIPEA (0.835 mL, 4.80 mmol, 5 eq), trans-cinnamic acid (170 mg, 1.15 mmol, 1.2 eq), HOBt (155 mg, 1.15 mmol, 1.2 eq), DMAP (catalytic amount) and EDC (220 mg, 1.15 mmol, 1.2 eq) were added and the resulting mixture was stirred at rt for 18 h. The reaction mixture was diluted with ethyl acetate (60 mL) and washed with a 1M HCl solution (2×30 mL), saturated NaHCO₃ solution (2×30 mL) and brine (30 mL). The organic layer was dried over anhydrous Na₂SO₄ and concentrated in vacuo to afford the subject compound as a white solid (313 mg, yield: 59%). ¹H NMR (400 MHz, DMSO-d₆) δ8.45 (d, 1H), 8.38 (t, 1H), 7.97 (d, 1H), 7.60-7.54 (m, 2H), 7.48-7.28 (m, 9H), 6.76 (d, 1H), 5.14 (d, 2H), 4.39-4.31 (m, 1H), 4.31-4.24 (m, 1H), 4.01 (q, 2H), 3.91 (d, 2H), 2.35 (t, 2H), 2.08-1.93 (m, 2H), 1.92-1.78 (m, 1H), 1.13 (t, 3H), 0.84 (t, 6H).

### Intermediate 47

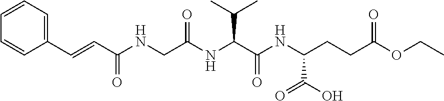 [SMILES: CCOC(=O)CC[C@@H](NC(=O)[C@@H](NC(=O)CNC(=O)/C=C/c1ccccc1)C(C)C)C(=O)O]

(R)-2-((S)-2-(2-cinnamamidoacetamido)-3-methylbutanamido)-5-ethoxy-5-oxopentanoic acid: To a solution of palladium (II) acetate (6 mg, 0.028 mmol, 0.05 eq) in dry DCM (5 mL) were added TEA (150 μL, 1.08 mmol, 1.95 eq) and triethylsilane (177 μL, 1.11 mmol, 2 eq). The resulting black solution was stirred for 15 minutes, after which a solution of Intermediate 46 (306 mg, 0.555 mmol, 1 eq) in dry DCM (7 mL) was added. The resulting mixture was stirred under argon atmosphere for 18 h, diluted with DCM (20 mL) and extracted with water (3×30 mL). Combined aqueous phases were acidified with a 1M HCl solution and extracted with DCM (2×30 mL). Combined organic phases were washed with water (30 mL) and brine (30 mL), dried over anhydrous Na₂SO₄ and concentrated in vacuo to afford the subject compound as a white solid (99 mg, yield: 39%). ¹H NMR (400 MHz, DMSO-d₆) δ12.67 (s, 1H), 8.37 (d, 1H), 8.28 (t, 1H), 7.93 (d, 1H), 7.57 (d, 2H), 7.47-7.33 (m, 4H), 6.75 (d, 1H), 4.32-4.17 (m, 2H), 4.08-3.97 (m, 2H), 3.91 (d, 2H), 2.33 (t, 2H), 2.06-1.90 (m, 2H), 1.88-1.74 (m, 1H), 1.15 (t, 3H), 0.88-0.79 (m, 6H).

### Compound 48

### SG189

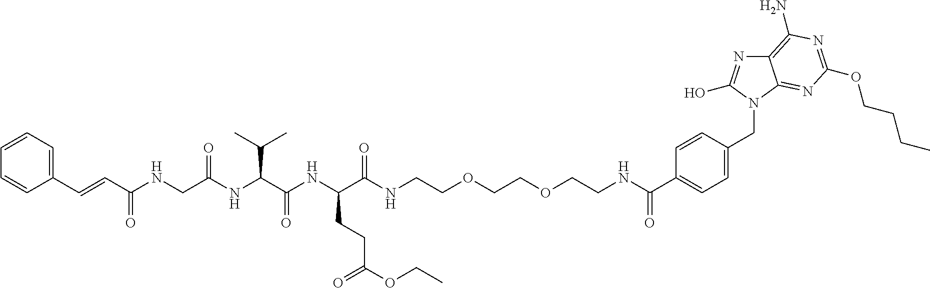 [SMILES: CCCCOc1nc(N)c2nc(O)n(Cc3ccc(C(=O)NCCOCCOCCNC(=O)[C@@H](CCC(=O)OCC)NC(=O)[C@@H](NC(=O)CNC(=O)/C=C/c4ccccc4)C(C)C)cc3)c2n1]

Ethyl (R)-1-(4-((6-amino-2-butoxy-8-hydroxy-9H-purin-9-yl)methyl)phenyl)-13-((S)-2-(2-cinnamamidoacetamido)-3-methylbutanamido)-1,12-dioxo-5,8- dioxa-2,11-diazahexadecan-16-oate: To an ice-chilled stirring solution of Intermediate 47 (63 mg, 0.14 mmol, 1 eq) in DMF (3 mL) were added DIPEA (71 μL, 0.41 mmol, 3 eq), Intermediate 29 (82 mg, 0.14 mmol, 1 eq) and COMU (64 mg, 0.15 mmol, 1.1 eq). The resulting mixture was stirred at rt for 20 h. Subsequently, 1 M HCl solution (40 mL) was added, after which the mixture was extracted with a mixture of DCM and isopropanol (3/1, 2×20 mL). The combined organic phases were washed with a saturated NaHCO₃ solution (2×40 mL) and brine (40 mL), dried over anhydrous Na₂SO₄ and concentrated in vacuo. The solid residue was washed with diethyl ether to afford the subject compound as an off-white solid (44 mg, yield: 32%). ¹H NMR (400 MHz, DMSO-d₆) δ10.00 (s, 1H), 8.53-8.42 (m, 1H), 8.38-8.24 (m, 2H), 7.96-7.86 (m, 2H), 7.78 (d, 2H), 7.60-7.53 (m, 2H), 7.46-7.36 (m, 4H), 7.34 (d, 2H), 6.73 (d, 1H), 6.48 (s, 2H), 4.90 (s, 2H), 4.28-4.19 (m, 1H), 4.17-4.09 (m, 3H), 4.07-3.97 (m, 2H), 3.91 (d, 2H), 3.54-3.44 (m, 6H), 3.42-3.35 (m, 4H), 3.28-3.10 (m, 2H), 2.32-2.24 (m, 2H), 2.01-1.87 (m, 2H), 1.81-1.67 (m, 1H), 1.66-1.54 (m, 2H), 1.42-1.29 (m, 2H), 1.15 (t, 3H), 0.94-0.79 (m, 9H). HRMS calculated for C₄₆H₆₃O₁₁N₁₀ m/z: 931.4683 (M+H)⁺, found 931.4700.

### Example 11: Compound SG190

### Intermediate 49

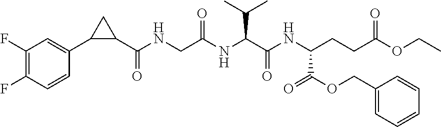 [SMILES: CCOC(=O)CC[C@@H](NC(=O)[C@@H](NC(=O)CNC(=O)C1CC1c1ccc(F)c(F)c1)C(C)C)C(=O)OCc1ccccc1]

1-benzyl 5-ethyl (2-(3,4-difluorophenyl)cyclopropane-1-carbonyl)glycyl-L-yalyl-D-glutamate: Intermediate 42 (500 mg, 0.96 mmol, 1 eq) was added to an ice-chilled stirred mixture of TFA and DCM (1/4, 4 mL), and the mixture was allowed to warm to room temperature. After 2 h, the solvent was evaporated in vacuo. The residue was coevaporated four times with diethyl ether and dissolved in DMF (5 mL). After cooling the solution in ice, DIPEA (0.84 mL, 4.79 mmol, 5 eq), 2-(3,4-difluorophenyl)cyclopropane-1-carboxylic acid (209 mg, 1.05 mmol, 1.1 eq), HOBt (142 mg, 1.05 mmol, 1.1 eq), DMAP (catalytic amount) and EDC (202 mg, 1.05 mmol, 1.1 eq) were added and the resulting mixture was stirred at rt for 18 h. The reaction mixture was diluted with ethyl acetate (60 mL) and washed with a 1 M HCl solution (2×30 mL), saturated NaHCO₃ solution (2×30 mL) and brine (30 mL). The organic layer was dried over anhydrous Na₂SO₄ and concentrated in vacuo to afford the subject compound as a white solid (432 mg, yield: 75%). ¹H NMR (400 MHz, DMSO-d₆) δ8.48-8.43 (m, 1H), 8.40 (t, 1H), 7.91-7.81 (m, 1H), 7.42-7.29 (m, 6H), 7.27-7.18 (m, 1H), 7.04-6.96 (m, 1H), 5.11 (d, 2H), 4.39-4.31 (m, 1H), 4.30-4.22 (m, 1H), 4.08-3.99 (m, 2H), 3.82 (d, 2H), 2.36 (t, 2H), 2.31-2.25 (m, 1H), 2.07-1.92 (m, 3H), 1.90-1.78 (m, 1H), 1.39-1.31 (m, 1H), 1.30-1.22 (m, 1H), 1.19-1.12 (m, 3H), 0.88-0.76 (m, 6H).

### Intermediate 50

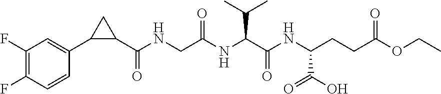 [SMILES: CCOC(=O)CC[C@@H](NC(=O)[C@@H](NC(=O)CNC(=O)C1CC1c1ccc(F)c(F)c1)C(C)C)C(=O)O]

(2R)-2-((2S)-2-(2-(2-(3,4-difluorophenyl)cyclopropane-1-carboxamido)acetamido)-3-methylbutanamido)-5-ethoxy-5-oxopentanoic acid: To a solution of palladium (II) acetate (30 mg, 0.13 mmol, 0.2 eq) in dry DCM (5 mL) were added TEA (186 μL, 1.34 mmol, 2 eq) and a solution of Intermediate 49 (411 mg, 0.67 mmol, 1 eq) in dry DCM (10 mL). After stirring for 5 minutes, triethylsilane (320 μL, 2.00 mmol, 3 eq) was added dropwise and the resulting mixture was stirred under argon atmosphere for 18 h. Following quenching by the addition of water (30 mL), the mixture was filtered, acidified with a 1M HCL solution and extracted with DCM (3×30 mL). Combined organic phases were washed with water (50 mL) and brine (50 mL), dried over anhydrous Na₂SO₄ and concentrated in vacuo to afford the subject compound as a brown solid (209 mg, yield: 61%). ¹H NMR (400 MHz, DMSO-d₆) δ8.49-8.38 (m, 1H), 8.20 (t, 1H), 7.90-7.81 (m, 1H), 7.38-7.28 (m, 1H), 7.28-7.18 (m, 1H), 7.05-6.97 (m, 1H), 4.31-4.22 (m, 1H), 4.22-4.13 (m, 1H), 4.09-3.97 (m, 2H), 3.81 (d, 2H), 2.38-2.24 (m, 3H), 2.06-1.91 (m, 3H), 1.88-1.72 (m, 1H), 1.38-1.31 (m, 1H), 1.29-1.21 (m, 1H), 1.20-1.13 (m, 3H), 0.83 (t, 6H).

### Compound 51

### SG190

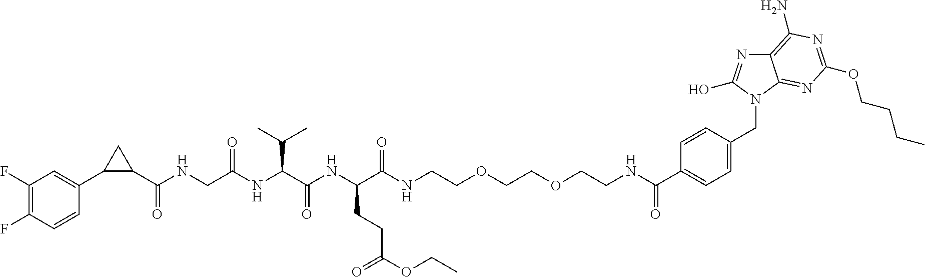 [SMILES: CCCCOc1nc(N)c2nc(O)n(Cc3ccc(C(=O)NCCOCCOCCNC(=O)[C@@H](CCC(=O)OCC)NC(=O)[C@@H](NC(=O)CNC(=O)C4CC4c4ccc(F)c(F)c4)C(C)C)cc3)c2n1]

Ethyl (13R)-1-(4-((6-amino-2-butoxy-8-hydroxy-9H-purin-9-yl)methyl)phenyl)-13-((2S)-2-(2-(2-(3,4-difluorophenyl)cyclopropane-1- carboxamido)acetamido)-3-methylbutanamido)-1,12-dioxo-5,8-dioxa-2,11-diazahexadecan-16-oate: To an ice-chilled stirring solution of Intermediate 50 (70 mg, mmol, 1 eq) in DMF (3 mL) were added DIPEA (71 μL, 0.41 mmol, 3 eq), Intermediate 29 (82 mg, mmol, 1 eq) and COM U (64 mg, 0.15 mmol, 1.1 eq). The resulting mixture was stirred at rt for 20 h. Subsequently, 1 M HCl solution (40 mL) was added, after which the mixture was extracted with a mixture of DCM and isopropanol (3/1, 2×30 mL). The combined organic phases were washed with a saturated NaHCO₃ solution (3×30 mL) and brine (30 mL), dried over anhydrous Na₂SO₄ and concentrated in vacuo. The solid residue was washed with diethyl ether to afford the subject compound as an off-white solid (22 mg, yield: 16%). ¹H NMR (400 MHz, DMSO-d₆) δ10.07 (s, 1H), 8.49 (t, 1H), 8.44-8.34 (m, 2H), 7.99-7.87 (m, 2H), 7.78 (d, 2H), 7.38-7.28 (m, 3H), 7.28-7.17 (m, 1H), 7.07-6.93 (m, 1H), 6.51 (s, 2H), 4.90 (s, 2H), 4.31-4.20 (m, 2H), 4.20-4.08 (m, 2H), 4.08-3.95 (m, 2H), 3.81 (d, 2H), 3.55-3.44 (m, 6H), 3.43-3.36 (m, 4H), 3.25-3.10 (m, 2H), 2.32-2.21 (m, 3H), 2.06-1.86 (m, 3H), 1.85-1.70 (m, 1H), 1.65-1.55 (m, 2H), 1.41-1.30 (m, 3H), 1.30-1.21 (m, 1H), 1.21-1.09 (m, 3H), 0.93-0.74 (m, 9H). HRMS calculated for C₄₇ H 63 0 11 N 10 F 2 m/z: 981.4640 (M +H)⁺, found 981.4608.

### Example 12: Compound SG196

### Intermediate 52

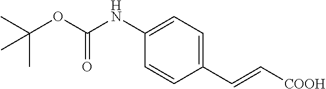 [SMILES: CC(C)(C)OC(=O)Nc1ccc(/C=C/C(=O)O)cc1]

(E)-3-(4-((tert-butoxycarbonyl)amino)phenyl)acrylic acid: To an ice-chilled stirring solution of 4-aminocinnamic acid (998 mg, 5.0 mmol) in water (4 mL) and 1 M NaOH (6.5 mL), a solution of di-tert-butyl dicarbonate (1.419 g, 6.5 mmol) in dioxane (20 mL) was added. The mixture was allowed to warm to room temperature and the stirring was continued overnight. Dioxane was evaporated in vacuo, after which the mixture was washed with diethyl ether (10 mL). The aqueous phase was acidified to pH=1 with a 1 M HCl solution and extracted with EtOAc (3×20 mL). The combined organic phases were dried over anhydrous Na₂SO₄ and concentrated in vacuo to afford the subject compound as an orange solid (1.062 g, 81%). ¹H NMR (400 MHz, DMSO-d₆) δ12.17 (s, 1H), 9.59 (s, 1H), 7.62-7.55 (m, 2H), 7.53-7.46 (m, 3H), 6.38 (d, 1H), 1.48 (s, 9H).

### Intermediate 53

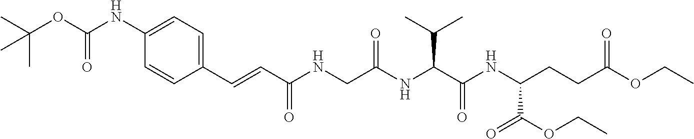 [SMILES: CCOC(=O)CC[C@@H](NC(=O)[C@@H](NC(=O)CNC(=O)/C=C/c1ccc(NC(=O)OC(C)(C)C)cc1)C(C)C)C(=O)OCC]

Diethyl ((E)-3-(4-((tert-butoxycarbonyl)amino)phenyl)acryloyl)glycyl-L-valyl-D-glutamate: Intermediate 5 (276 mg, 0.60 mmol, 1 eq) was added to an ice-chilled stirred mixture of TFA and DCM (1/5, 4 mL), and the mixture was allowed to warm to room temperature. After 3 h, the solvent was evaporated in vacuo. The residue was coevaporated three times with diethyl ether and dissolved in DMF (3 mL). After cooling the solution in ice, DIPEA (0.52 mL, 3.0 mmol, 5 eq), Intermediate 52 (174 mg, 0.66 mmol, 1.1 eq), HOBt (89 mg, 0.66 mmol, 1.1 eq), DMAP (catalytic amount) and EDC (127 mg, 0.66 mmol, 1.1 eq) were added and the resulting mixture was stirred at rt for 18 h. The reaction mixture was diluted with ethyl acetate (40 mL) and washed with a 1 M HCl solution (2×20 mL), saturated NaHCO₃ solution (2×20 mL) and brine (20 mL). The organic layer was dried over anhydrous Na₂SO₄ and concentrated in vacuo to afford the subject compound as an orange solid (179 mg, yield: 49%), which was used in the next step without any further purification.

### Intermediate 54

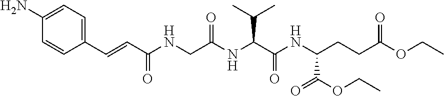 [SMILES: CCOC(=O)CC[C@@H](NC(=O)[C@@H](NC(=O)CNC(=O)/C=C/c1ccc(N)cc1)C(C)C)C(=O)OCC]

Diethyl ((E)-3-(4-aminophenyl)acryloyl)glycyl-L-valyl-D-glutamate: Intermediate 53 (179 mg, 0.30 mmol) was added to an ice-chilled stirred mixture of TFA and DCM (1/5, 4 mL), and the mixture was allowed to warm to room temperature. After 3 h, the solvent was evaporated in vacuo. The residue was dissolved in EtOAc (50 mL) and washed with a saturated NaHCO₃ solution (3×30 mL) and brine (30 mL). The organic layer was dried over anhydrous Na₂SO₄ and concentrated in vacuo to afford the subject compound as an orange solid (135 mg, 91%). ¹H NMR (400 MHz, MeOD) 6 7.46 (d, 1H), 7.34 (d, 2H), 6.70 (d, 2H), 6.42 (d, 1H), 4.48-4.42 (m, 1H), 4.31 (d, 1H), 4.23-4.14 (m, 2H), 4.14-3.92 (m, 4H), 2.43 (t, 2H), 2.27-2.13 (m, 2H), 2.08-1.95 (m, 1H), 1.28 (t, 3H), 1.22 (t, 3H), 0.99 (t, 6H).

Intermediate 55

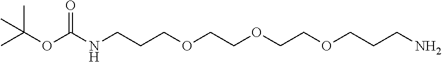 [SMILES: CC(C)(C)OC(=O)NCCCOCCOCCOCCCN]

Tert-butyl (3-(2-(2-(3-aminopropoxy)ethoxy)ethoxy)propyl)carbamate: A solution of Boc₂O (4.365 g, 20 mmol, 1 eq) in DCM (40 mL) was added dropwise to a stirring solution of diethylene glycol bis(3-aminopropyl) ether (22.021 g, 100 mmol, 5 eq) in DCM (100 mL) at 0° C. The resulting mixture was stirred at rt for 20 h, after which the solvent was evaporated in vacuo. The residue was dissolved in a saturated NaHCO₃ solution (50 mL) and extracted with DCM (2×50 mL). The combined organic phases were washed with a saturated NaHCO₃ solution (2×50 mL) and brine (100 mL), dried over anhydrous Na₂SO₄ and concentrated in vacuo to afford the subject compound as a colourless oil (3.870 g, yield: 60%). ¹H NMR (400 MHz, DMSO-d₆) δ6.82-6.72 (m, 1H), 3.53-3.34 (m, 12H), 2.96 (q, 2H), 2.57 (t, 2H), 1.63-1.51 (m, 4H), 1.37 (s, 9H).

### Intermediate 56

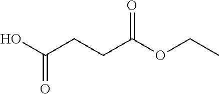

4-ethoxy-4-oxobutanoic acid: Succinic anhydride (5 g, 50 mmol) was suspended in anhydrous ethanol (15 mL) and refluxed for 2 h, after which the mixture was concentrated in vacuo to afford the subject compound as a colourless oil (7.31 g, yield: 100%). ¹H NMR (400 MHz, CDCl₃) 6 10.98 (s, 1H), 4.16 (q, 2H), 2.71-2.66 (m, 2H), 2.64-2.60 (m, 2H), 1.26 (t, 3H).

### Intermediate 57

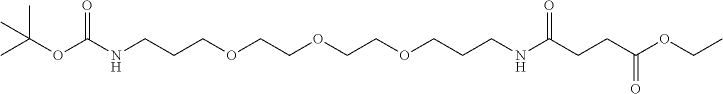 [SMILES: CCOC(=O)CCC(=O)NCCCOCCOCCOCCCNC(=O)OC(C)(C)C]

Ethyl 2,2-dimethyl-4,20-dioxo-3,9,12,15-tetraoxa-5,19-diazatricosan-23-oate: To an ice-chilled stirring solution of Intermediate 55 (3.870 g, 12.1 mmol, 1 eq) in DCM (40 mL) were added Intermediate 56 (1.942 g, 13.3 mmol, 1.1 eq), DIPEA (6.31 mL, 36.2 mmol, 3 eq), HOBt (1.795 g, 13.3 mmol, 1.1 eq), DMAP (catalytic amount) and EDC (2.548 g, 13.3 mmol, 1.1 eq) and the resulting mixture was stirred at rt for 18 h. The reaction mixture was diluted with DCM (30 mL) and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as yellow oil (4.471 g, yield: 83%). ¹H NMR (400 MHz, DMSO-d₆) δ7.83 (t, 1H), 6.76 (t, 1H), 4.03 (q, 2H), 3.54-3.49 (m, 4H), 3.48-3.44 (m, 4H), 3.41-3.35 (m, 4H), 3.07 (q, 2H), 2.96 (q, 2H), 2.50-2.45 (m, 2H), 2.33 (t, 2H), 1.65-1.54 (m, 4H), 1.37 (s, 9H), 1.17 (t, 3H).

### Intermediate 58

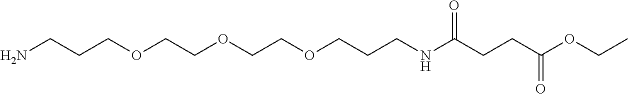 [SMILES: CCOC(=O)CCC(=O)NCCCOCCOCCOCCCN]

Ethyl 1-amino-15-oxo-4,7,10-trioxa-14-azaoctadecan-18-oate: Intermediate 57 (4.41 g, 9.83 mmol) was added to an ice-chilled stirred mixture of TFA and DCM (1/5, 25 mL), and the mixture was allowed to warm to room temperature. After 4 h, the solvent was evaporated in vacuo. The residue was coevaporated three times with diethyl ether to afford the subject compound as a yellow oil (4.55 g, yield: 100%). ¹H NMR (400 MHz, DMSO-d₆) δ7.86 (t, 1H), 7.70 (s, 2H), 4.03 (q, 2H), 3.55-3.43 (m, 10H), 3.38 (t, 2H), 3.07 (q, 2H), 2.91-2.80 (m, 2H), 2.55-2.45 (m, 2H), 2.37-2.28 (m, 2H), 1.77 (p, 2H), 1.60 (p, 2H), 1.17 (t, 3H).

### Intermediate 59

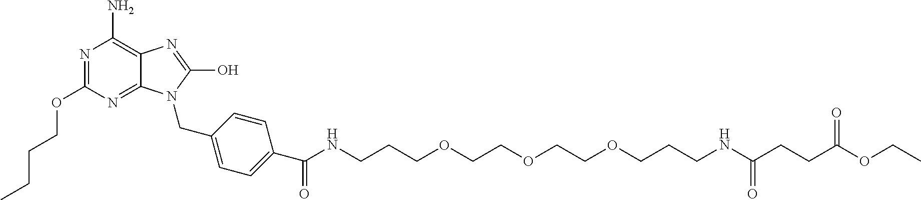 [SMILES: CCCCOc1nc(N)c2nc(O)n(Cc3ccc(C(=O)NCCCOCCOCCOCCCNC(=O)CCC(=O)OCC)cc3)c2n1]

Ethyl 1-(4-((6-amino-2-butoxy-8-hydroxy-9H-purin-9-yl)methyl)phenyl)-1,17-dioxo-6,9,12-trioxa-2,16-diazaicosan-20-oate: Compound 10 (0.427 g, 1.20 mmol, 1 eq) was dissolved in DMSO (20 mL). Intermediate 58 (1.658 g, 3.59 mmol, 3 eq) and DIPEA (2.08 mL, 11.95 mmol, 10 eq) were dissolved in DCM (1 mL) and added to the stirring solution of Compound 10 in DMSO. After cooling the reaction mixture in ice, COMU (1.279 g, 2.99 mmol, 2.5 eq) was added and the mixture was stirred at rt for 20 h. Ethyl acetate (100 mL) and a 1 M NaHCO₃ solution (75 mL) were added. After concentrating the mixture in vacuo, it was cooled on ice for one hour. The precipitate was filtered and washed with water and diethyl ether to afford the subject compound (271 mg, yield: 33%), which was used in the next step without any further purification.

### Intermediate 60

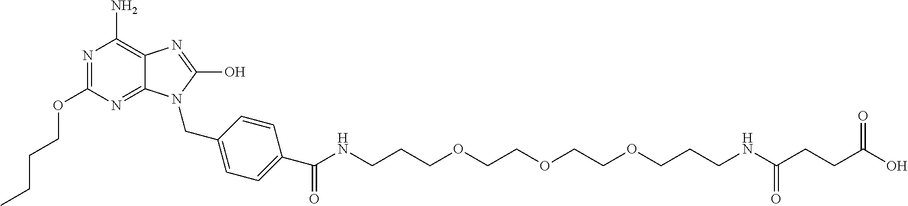 [SMILES: CCCCOc1nc(N)c2nc(O)n(Cc3ccc(C(=O)NCCCOCCOCCOCCCNC(=O)CCC(=O)O)cc3)c2n1]

1-(4-((6-amino-2-butoxy-8-hydroxy-9H-purin-9-yl)methyl)phenyl)-1,17-dioxo-6,9,12-trioxa-2,16-diazaicosan-20-oic acid: To a stirring solution of Intermediate 59 (271 mg, 0.39 mmol) in methanol (20 mL) was added 1M aqueous NaOH (4 mL). The mixture was stirred at rt for 3 h. Water (6 mL) was added and the mixture was acidified using a 1M HCl solution. Methanol was evaporated in vacuo and the resulting suspension was cooled on ice for 1 h. The obtained precipitate was filtered and washed with water and diethyl ether to afford the subject compound as an off-white solid (207 mg, yield 80%). ¹H NMR (400 MHz, DMSO-d₆) δ12.04 (s, 1H), 9.99 (s, 1H), 8.39 (t, 1H), 7.84-7.71 (m, 3H), 7.34 (d, 2H), 6.47 (s, 2H), 4.90 (s, 2H), 4.13 (t, 2H), 3.55-3.40 (m, 10H), 3.39-3.25 (m, 4H), 3.06 (q, 2H), 2.40 (t, 2H), 2.28 (t, 2H), 1.80-1.68 (m, 2H), 1.66-1.53 (m, 4H), 1.43-1.28 (m, 2H), 0.89 (t, 3H).

### Compound 61

### SG196

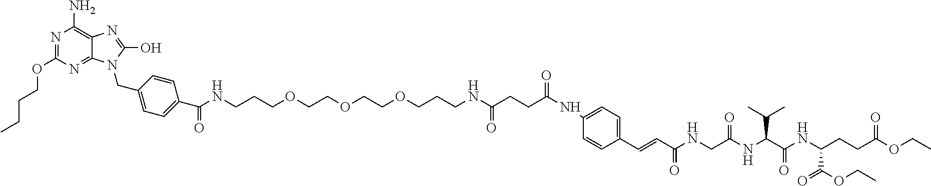 [SMILES: CCCCOc1nc(N)c2nc(O)n(Cc3ccc(C(=O)NCCCOCCOCCOCCCNC(=O)CCC(=O)Nc4ccc(/C=C/C(=O)NCC(=O)N[C@H](C(=O)N[C@H](CCC(=O)OCC)C(=O)OCC)C(C)C)cc4)cc3)c2n1]

Diethyl ((E)-3-(4-(1-(4-((6-amino-2-butoxy-8-hydroxy-9H-purin-9-yl)methyl)phenyl)-1,17-dioxo-6,9,12-trioxa-2,16-diazaicosan-20- amido)phenyl)acryloyl)glycyl-L-yalyl-D-glutamate: To an ice-chilled stirring solution of Intermediate 60 (33 mg, 0.05 mmol, 1 eq) in DMF (2 mL) were added DIPEA (26 μL, 0.15 mmol, 3 eq), Intermediate 54 (28 mg, 0.055 mmol, 1.1 eq) and COMU (24 mg, 0.055 mmol, 1.1 eq). The resulting mixture was stirred at rt for 40 h, after which it was diluted with a mixture of DCM and isopropanol (3/1, 30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo. The solid residue was washed twice with diethyl ether to afford the subject compound as an off-white solid (25 mg, yield: 44%). ¹H NMR (400 MHz, DMSO-d₆) δ9.99 (s, 1H), 8.45-8.37 (m, 2H), 8.33 (t, 1H), 7.92 (d, 1H), 7.87 (t, 1H), 7.82-7.73 (m, 3H), 7.63 (d, 2H), 7.50 (d, 2H), 7.39-7.31 (m, 3H), 6.62 (d, 1H), 6.48 (s, 2H), 4.90 (s, 2H), 4.30-4.21 (m, 2H), 4.13 (t, 2H), 4.10-3.98 (m, 4H), 3.89 (d, 2H), 3.54-3.25 (m, 14H), 3.07 (t, 2H), 2.43-2.29 (m, 6H), 2.04-1.93 (m, 2H), 1.88-1.79 (m, 1H), 1.79-1.68 (m, 2H), 1.66-1.55 (m, 4H), 1.43-1.29 (m, 2H), 1.21-1.12 (m, 6H), 0.93-0.80 (m, 9H).

### Example 13: Compound SG197

### Intermediate 62

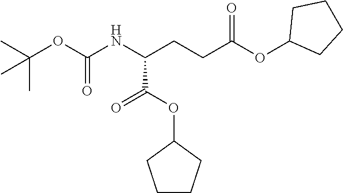 [SMILES: CC(C)(C)OC(=O)N[C@H](CCC(=O)OC1CCCC1)C(=O)OC1CCCC1]

Dicyclopentyl (tert-butoxycarbonyl)-D-glutamate: To an ice-chilled stirring solution of N-Boc-D-glutamic acid (495 mg, 2.0 mmol, 1 eq) in DCM (15 ml), cyclopentanol (0.727 mL, 8 mmol, 8 eq), EDC (844 mg, 4.4 mmol, 4.4 eq), and DMAP (65 mg, 0.53 mmol, 0.53 eq) were added. The mixture was allowed to warm to room temperature, and the stirring was continued overnight. The solvent was evaporated in vacuo. The residue was dissolved in EtOAc (25 mL) and washed with a 1 M HCl solution (2×25 mL), saturated NaHCO₃ solution (2×25 mL) and brine (25 mL). The organic layer was dried over anhydrous Na₂SO₄ and concentrated in vacuo to afford the subject compound as a colourless oil (625 mg, 82%). ¹H NMR (400 MHz, CDCI 3) δ5.25-5.13 (m, 2H), 5.09 (d, 1H), 4.29-4.20 (m, 1H), 2.44-2.25 (m, 2H), 2.20-2.06 (m, 1H), 1.95-1.82 (m, 5H), 1.79-1.64 (m, 8H), 1.64-1.53 (m, 4H), 1.44 (s, 9H).

### Intermediate 63

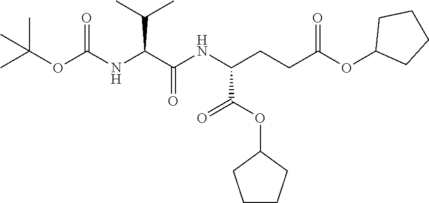 [SMILES: CC(C)[C@H](NC(=O)OC(C)(C)C)C(=O)N[C@H](CCC(=O)OC1CCCC1)C(=O)OC1CCCC1]

Dicyclopentyl (tert-butoxycarbonyl)-L-valyl-D-glutamate: Intermediate 62 (591 mg, 1.54 mmol, 1 eq) was added to an ice-chilled stirred mixture of TFA and DCM (1/3, 5 mL), and the mixture was allowed to warm to room temperature. After 3 h, the solvent was evaporated in vacuo. The residue was coevaporated four times with diethyl ether and dissolved in DMF (4 mL). After cooling the solution in ice, DIPEA (1.34 mL, 7.7 mmol, 5 eq), Intermediate 1 (368 mg, 1.70 mmol, 1.1 eq), HOBt (230 mg, 1.70 mmol, 1.1 eq), DMAP (catalytic amount) and EDC (326 mg, 1.70 mmol, 1.1 eq) were added and the resulting mixture was stirred at rt for 18 h. The reaction mixture was diluted with ethyl acetate (60 mL) and washed with a 1 M HCl solution (2×30 mL), saturated NaHCO₃ solution (2×30 mL) and brine (30 mL). The organic layer was dried over anhydrous Na₂SO₄ and concentrated in vacuo to afford the subject compound as a yellow oil (494 mg, yield: 67%). ¹H NMR (400 MHz, CDCl₃) δ6.69 (d, 1H), 5.25-5.11 (m, 2H), 5.00 (s, 1H), 4.58-4.49 (m, 1H), 3.99 (s, 1H), 2.43-2.25 (m, 2H), 2.23-2.10 (m, 2H), 2.02-1.89 (m, 1H), 1.88-1.78 (m, 4H), 1.75-1.64 (m, 8H), 1.63-1.54 (m, 4H), 1.45 (s, 9H), 0.97 (d, 3H), 0.91 (d, 3H).

### Intermediate 64

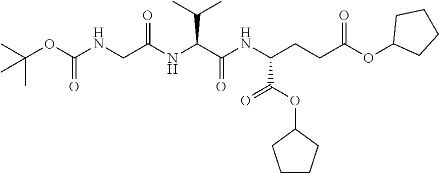 [SMILES: CC(C)[C@H](NC(=O)CNC(=O)OC(C)(C)C)C(=O)N[C@H](CCC(=O)OC1CCCC1)C(=O)OC1CCCC1]

Dicyclopentyl (tert-butoxycarbonyl)glycyl-L-valyl-D-glutamate: Intermediate 63 (462 mg, 0.96 mmol, 1 eq) was added to an ice-chilled stirred mixture of TFA and DCM (1/3, 5 mL), and the mixture was allowed to warm to room temperature. After 3 h, the solvent was evaporated in vacuo. The residue was coevaporated four times with diethyl ether and dissolved in DMF (4 mL). After cooling the solution in ice, DIPEA (0.84 mL, 4.8 mmol, 5 eq), Intermediate 2 (184 mg, 1.05 mmol, 1.1 eq), HOBt (142 mg, 1.05 mmol, 1.1 eq), DMAP (catalytic amount) and EDC (201 mg, 1.05 mmol, 1.1 eq) were added and the resulting mixture was stirred at rt for 18 h. The reaction mixture was diluted with ethyl acetate (50 mL) and washed with a 1 M HCl solution (2×25 mL), saturated NaHCO₃ solution (2×25 mL) and brine (25 mL). The organic layer was dried over anhydrous Na₂SO₄ and concentrated in vacuo to afford the subject compound as white solid (330 mg, yield: 64%). ¹H NMR (400 MHz, CDCl₃) δ6.99 (d, 1H), 6.61 (d, 1H), 5.27-5.09 (m, 3H), 4.46 (td, 1H), 4.36 (dd, 1H), 3.90 (dd, 1H), 3.79 (dd, 1H), 2.35 (q 2H), 2.31-2.22 (m, 1H), 2.20-2.07 (m, 1H), 2.06-1.92 (m, 1H), 1.90-1.79 (m, 4H), 1.77-1.54 (m, 12H), 1.46 (s, 9H), 0.97 (d, 3H), 0.93 (d, 3H).

### Intermediate 65

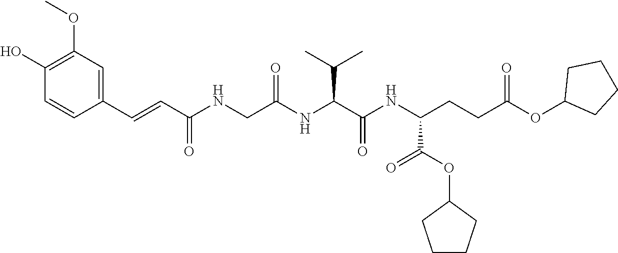 [SMILES: COc1cc(/C=C/C(=O)NCC(=O)N[C@H](C(=O)N[C@H](CCC(=O)OC2CCCC2)C(=O)OC2CCCC2)C(C)C)ccc1O]

Dicyclopentyl ((E)-3-(4-hydroxy-3-methoxyphenyl)acryloyl)glycyl-L-yalyl-D-glutamate: Intermediate 64 (108 mg, 0.20 mmol, 1 eq) was added to an ice-chilled stirred mixture of TFA and DCM (1/3, 4 mL), and the mixture was allowed to warm to room temperature. After 3 h, the solvent was evaporated in vacuo. The residue was coevaporated four times with diethyl ether and dissolved in DMF (3 mL). After cooling the solution in ice, DIPEA (0.174 mL, 1.0 mmol, 5 eq), trans-ferulic acid (43 mg, 0.22 mmol, 1.1 eq), HOBt (30 mg, 0.22 mmol, 1.1 eq), DMAP (catalytic amount) and EDC (42 mg, 0.22 mmol, 1.1 eq) were added and the resulting mixture was stirred at rt for 18 h. The reaction mixture was diluted with ethyl acetate (30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo to afford the subject compound as white solid (93 mg, yield: 76%). ¹H NMR (400 MHz, CDCl₃) δ7.56 (d, 1H), 7.24 (d, 1H), 7.09-6.97 (m, 3H), 6.92-6.81 (m, 2H), 6.37 (d, 1H), 6.00 (s, 1H), 5.21-5.07 (m, 2H), 4.55-4.47 (m, 1H), 4.46-4.40 (m, 1H), 4.23-4.06 (m, 2H), 3.91 (s, 3H), 2.41-2.29 (m, 2H), 2.26-2.08 (m, 2H), 2.06-1.93 (m, 1H), 1.87-1.76 (m, 4H), 1.74-1.51 (m, 12H), 1.00-0.88 (m, 6H).

### Compound 66

### SG197

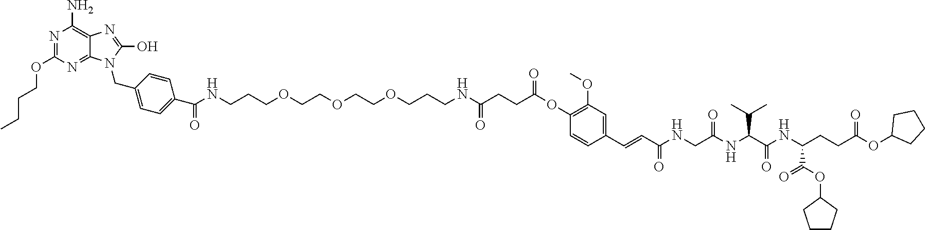 [SMILES: CCCCOc1nc(N)c2nc(O)n(Cc3ccc(C(=O)NCCCOCCOCCOCCCNC(=O)CCC(=O)Oc4ccc(/C=C/C(=O)NCC(=O)N[C@H](C(=O)N[C@H](CCC(=O)OC5CCCC5)C(=O)OC5CCCC5)C(C)C)cc4OC)cc3)c2n1]

Dicyclopentyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3-methoxyphenyl)acryloyl)glycyl-L-yalyl-D-glutamate: To an ice-chilled stirring solution of Intermediate 60 (19 mg, 0.029 mmol, 1.2 eq) in DMF (2 mL) were added DIPEA (13 μL, 0.073 mmol, 3 eq), Intermediate 65 (15 mg, 0.024 mmol, 1 eq) and COMU (13 mg, 0.029 mmol, 1.2 eq). The resulting mixture was stirred at rt for 18 h, after which it was diluted with a mixture of DCM and isopropanol (3/1, 30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo. The solid residue was washed twice with diethyl ether to afford the subject compound as an off-white solid (9 mg, yield: 29%). ¹H NMR (400 MHz, DMSO-d₆) δ9.98 (s, 1H), 8.42-8.36 (m, 1H), 8.31 (t, 1H), 7.96 (d, 1H), 7.86 (t, 1H), 7.81-7.73 (m, 3H), 7.51 (d, 1H), 7.48-7.38 (m, 3H), 7.34 (d, 2H), 6.84 (d, 1H), 6.47 (s, 2H), 5.10-5.00 (m, 2H), 4.90 (s, 2H), 4.30-4.16 (m, 2H), 4.13 (t, 2H), 3.96 (s, 3H), 3.92 (d, 2H), 3.52-3.37 (m, 10H), 3.32-3.22 (m, 4H), 3.15-3.03 (m, 2H), 2.48-2.39 (m, 2H), 2.39-2.25 (m, 4H), 2.01-1.88 (m, 2H), 1.86-1.68 (m, 7H), 1.66-1.48 (m, 16H), 1.45-1.30 (m, 2H), 0.93-0.82 (m, 9H).

### Example 14: Compound SG198

### Intermediate 67

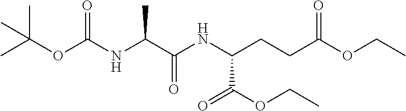 [SMILES: CCOC(=O)CC[C@@H](NC(=O)[C@H](C)NC(=O)OC(C)(C)C)C(=O)OCC]

Diethyl (tert-butoxycarbonyl)-L-alanyl-D-glutamate: To an ice-chilled stirring solution of Boc-L-alanine (1.457 g, 7.7 mmol, 1.1 eq) in ethyl acetate (50 mL) were added HOBt (1.040 g, 7.7 mmol, 1.1 eq) and DCC (1.589 g, 7.7 mmol, 1.1 eq). After stirring for 30 minutes, Intermediate 3 (1.678 g, 7 mmol, 1 eq), DIPEA (3.05 mL, 17.5 mmol, 2.5 eq) and DMAP (catalytic amount) were added and the resulting mixture was stirred at rt for 18 h, after which hexane (20 mL) was added. The mixture was filtered and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as an off-white crystalline solid (2.149 g, yield: 82%), which was used in the next step without any further purification.

### Intermediate 68

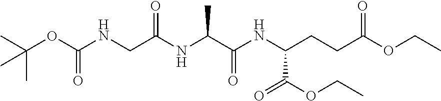 [SMILES: CCOC(=O)CC[C@@H](NC(=O)[C@H](C)NC(=O)CNC(=O)OC(C)(C)C)C(=O)OCC]

Diethyl (tert-butoxycarbonyl)glycyl-L-alanyl-D-glutamate: Intermediate 67 (1.872 g, 5.0 mmol) was added to an ice-chilled stirred mixture of TFA and DCM (1/5, 20 mL), and the mixture was allowed to warm to room temperature. After 3 h, the solvent was evaporated in vacuo. The residue was coevaporated three times with diethyl ether. Concurrently, HOBt (0.743 g, 5.5 mmol, 1.1 eq), DCC (1.135 g, 5.5 mmol, 1.1 eq) and DMAP (catalytic amount) were added to an ice-chilled stirring solution of Intermediate 2 (0.963 g, 5.5 mmol, 1.1 eq) in ethyl acetate (30 mL). After 30 minutes, a solution of the deprotected Intermediate 67 in ethyl acetate (20 mL) and DIPEA (4.35 mL, 25.0 mmol, 5 eq) was added and the resulting mixture was stirred at rt for 18 h. Hexane (20 mL) was added, after which the mixture was filtered and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as an off-white crystalline solid (1.93 g, yield: 90%). ¹H NMR (400 MHz, CDCl₃) δ7.16 (d, 1H), 6.73 (d, 1H), 5.29 (s, 1H), 4.60-4.49 (m, 2H), 4.21-4.10 (m, 4H), 3.90-3.74 (m, 2H), 2.46-2.33 (m, 2H), 2.23-2.15 (m, 1H), 2.06-1.96 (m, 1H), 1.45 (s, 9H), 1.40 (d, 3H), 1.28-1.23 (m, 6H).

### Intermediate 69

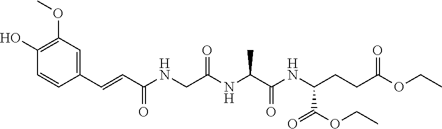 [SMILES: CCOC(=O)CC[C@@H](NC(=O)[C@H](C)NC(=O)CNC(=O)/C=C/c1ccc(O)c(OC)c1)C(=O)OCC]

Diethyl ((E)-3-(4-hydroxy-3-methoxyphenyl)acryloyI)-glycyl-L-alanyl-D-glutamate: Intermediate 68 (129 mg, 0.3 mmol, 1 eq) was added to an ice-chilled stirred mixture of TFA and DCM (1/5, 4 mL), and the mixture was allowed to warm to room temperature. After 3 h, the solvent was evaporated in vacuo. The residue was coevaporated three times with diethyl ether and dissolved in DMF (3 mL). After cooling the solution in ice, DIPEA (0.261 mL, 1.5 mmol, 5 eq), trans-ferulic acid (64 mg, 0.33 mmol, 1.1 eq), HOBt (45 mg, 0.33 mmol, 1.1 eq), DMAP (catalytic amount) and EDC (63 mg, 0.33 mmol, 1.1 eq) were added and the resulting mixture was stirred at rt for 18 h. The reaction mixture was diluted with ethyl acetate (30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo to afford the subject compound a white solid (66 mg, yield: 43%). ¹H NMR (400 MHz, CDCl₃) δ7.54 (d, 1H), 7.18 (d, 1H), 7.06 (dd, 1H), 7.01 (d, 1H), 6.92-6.88 (m, 2H), 6.61 (t, 1H), 6.33 (d, 1H), 5.91 (s, 1H), 4.62-4.51 (m, 1H), 4.22-4.00 (m, 6H), 3.91 (s, 3H), 2.45-2.33 (m, 2H), 2.25-2.17 (m, 1H), 2.08-1.99 (m, 1H), 1.42 (d, 3H), 1.28-1.21 (m, 6H).

### Compound 70

### SG198

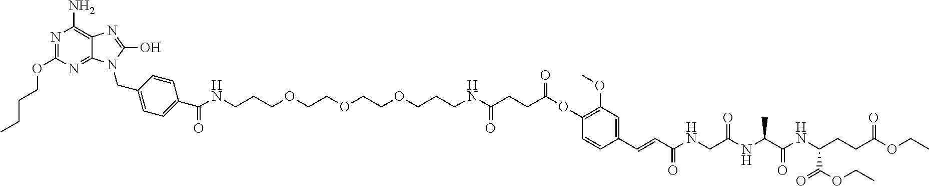 [SMILES: CCCCOc1nc(N)c2nc(O)n(Cc3ccc(C(=O)NCCCOCCOCCOCCCNC(=O)CCC(=O)Oc4ccc(/C=C/C(=O)NCC(=O)N[C@@H](C)C(=O)N[C@H](CCC(=O)OCC)C(=O)OCC)cc4OC)cc3)c2n1]

Diethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3- methoxyphenyl)acryloyl)glycyl-L-alanyl-D-glutamate: To an ice-chilled stirring solution of Intermediate 60 (42 mg, 0.064 mmol, 1.2 eq) in DMF (2 mL) were added DIPEA (28 μL, 0.16 mmol, 3 eq), Intermediate 69 (27 mg, 0.053 mmol, 1 eq) and COMU (27 mg, 0.064 mmol, 1.2 eq). The resulting mixture was stirred at rt for 18 h, after which it was diluted with a mixture of DCM and isopropanol (3/1, 30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo. The solid residue was washed twice with diethyl ether to afford the subject compound as an off-white solid (32 mg, yield: 52%). ¹H NMR (400 MHz, DMSO-d₆) δ10.01 (s, 1H), 8.46-8.31 (m, 2H), 8.28-8.19 (m, 1H), 7.96-7.87 (m, 1H), 7.80-7.73 (m, 3H), 7.47-7.39 (m, 2H), 7.34 (d, 2H), 7.17 (dd, 1H), 7.09 (d, 1H), 6.75 (d, 1H), 6.48 (s, 2H), 4.90 (s, 2H), 4.39-4.29 (m, 1H), 4.29-4.22 (m, 1H), 4.16-3.97 (m, 6H), 3.85 (d, 2H), 3.81 (s, 3H), 3.55-3.40 (m, 3.32-3.23 (m, 4H), 3.15-3.03 (m, 2H), 2.48-2.41 (m, 2H), 2.37-2.30 (m, 4H), 2.07-1.97 (m, 1H), 1.94-1.81 (m, 1H), 1.77-1.69 (m, 2H), 1.66-1.57 (m, 4H), 1.42-1.30 (m, 2H), 1.24 (d, 3H), 1.21-1.11 (m, 6H), 0.95-0.84 (m, 3H).

### Example 15: Compound SG199

### Intermediate 71

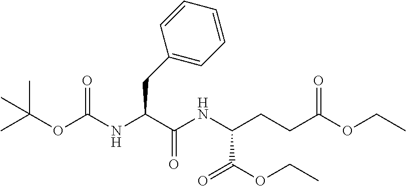 [SMILES: CCOC(=O)CC[C@@H](NC(=O)[C@H](Cc1ccccc1)NC(=O)OC(C)(C)C)C(=O)OCC]

Diethyl (tert-butoxycarbonyl)-L-phenylalanyl-D-glutamate: To an ice-chilled stirring solution of Boc-L-phenylalanine (2.043 g, 7.7 mmol, 1.1 eq) in ethyl acetate (50 mL) were added HOBt (1.040 g, 7.7 mmol, 1.1 eq) and DCC (1.589 g, 7.7 mmol, 1.1 eq). After stirring for 30 minutes, Intermediate 3 (1.678 g, 7 mmol, 1 eq), DIPEA (3.05 mL, 17.5 mmol, 2.5 eq) and DMAP (catalytic amount) were added and the resulting mixture was stirred at rt for 18 h, after which hexane (20 mL) was added. The mixture was filtered and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as an off-white crystalline solid (2.397 g, yield: 76%), which was used in the next step without any further purification.

### Intermediate 72

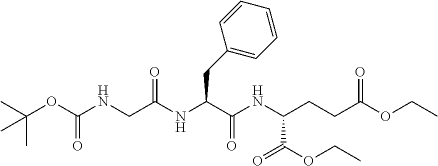 [SMILES: CCOC(=O)CC[C@@H](NC(=O)[C@H](Cc1ccccc1)NC(=O)CNC(=O)OC(C)(C)C)C(=O)OCC]

Diethyl (tert-butoxycarbonyl)glycyl-L-phenylalanyl-D-glutamate: Intermediate 71 (2.253 g, 5.0 mmol) was added to an ice-chilled stirred mixture of TFA and DCM (1/5, 20 mL), and the mixture was allowed to warm to room temperature. After 3 h, the solvent was evaporated in vacuo. The residue was coevaporated three times with diethyl ether. Concurrently, HOBt (0.743 g, 5.5 mmol, 1.1 eq), DCC (1.135 g, 5.5 mmol, 1.1 eq) and DMAP (catalytic amount) were added to an ice-chilled stirring solution of Intermediate 2 (0.963 g, 5.5 mmol, 1.1 eq) in ethyl acetate (30 mL). After 30 minutes, a solution of the deprotected Intermediate 71 in ethyl acetate (20 mL) and DIPEA (4.35 mL, 25.0 mmol, 5 eq) was added and the resulting mixture was stirred at rt for 18 h. Hexane (20 mL) was added, after which the mixture was filtered and washed with a 1 M HCl solution (2×50 mL), saturated NaHCO₃ solution (2×50 mL) and brine (50 mL). The organic layer was dried over anhydrous Na₂SO₄ and concentrated in vacuo to afford the subject compound as a white solid (1.93 g, yield: 76%). ¹H NMR (400 MHz, CDCl₃) δ7.31-7.20 (m, 5H), 6.76 (d, 1H), 6.61 (d, 1H), 5.14 (s, 1H), 4.76-4.70 (m, 1H), 4.48-4.43 (m, 1H), 4.19-4.08 (m, 4H), 3.85-3.73 (m, 2H), 3.16-3.02 (m, 2H), 2.20-2.16 (m, 2H), 2.07-1.99 (m, 1H), 1.91-1.82 (m, 1H), 1.43 (s, 9H), 1.28-1.23 (m, 6H).

### Intermediate 73

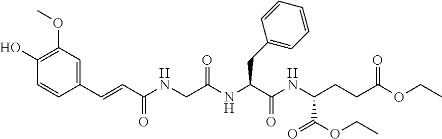 [SMILES: CCOC(=O)CC[C@@H](NC(=O)[C@H](Cc1ccccc1)NC(=O)CNC(=O)/C=C/c1ccc(O)c(OC)c1)C(=O)OCC]

Diethyl ((E)-3-(4-Hydroxy-3-methoxyphenyl)acryloyI)-glycyl-L-phenylalanyl-D-glutamate: Intermediate 72 (152 mg, 0.3 mmol, 1 eq) was added to an ice-chilled stirred mixture of TFA and DCM (1/5, 4 mL), and the mixture was allowed to warm to room temperature. After 3 h, the solvent was evaporated in vacuo. The residue was coevaporated three times with diethyl ether and dissolved in DMF (3 mL). After cooling the solution in ice, DIPEA (0.261 mL, 1.5 mmol, 5 eq), trans-ferulic acid (64 mg, 0.33 mmol, 1.1 eq), HOBt (45 mg, 0.33 mmol, 1.1 eq), DMAP (catalytic amount) and EDC (63 mg, 0.33 mmol, 1.1 eq) were added and the resulting mixture was stirred at rt for 18 h. The reaction mixture was diluted with ethyl acetate (30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo to afford the subject compound an off-white solid (84 mg, yield: 48%). ¹H NMR (400 MHz, CDCl₃) δ7.66-7.46 (m, 3H), 7.23-6.92 (m, 8H), 6.85 (d, 1H), 6.36 (d, 1H), 4.92-4.86 (m, 1H), 4.52-4.47 (m, 1H), 4.13-4.03 (m, 6H), 3.83 (s, 3H), 3.14-3.02 (m, 2H), 2.14-2.09(m, 2H), 2.06-1.98 (m, 1H), 1.93-1.83(m, 1H), 1.22-1.18 (m, 6H).

### Compound 74

### SG199

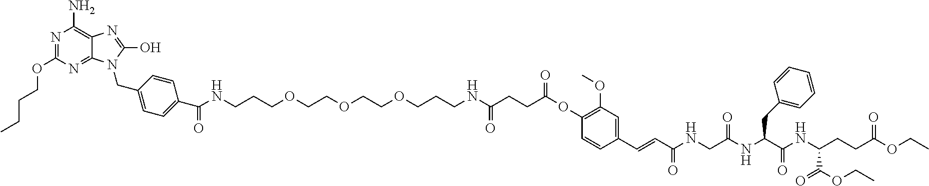 [SMILES: CCCCOc1nc(N)c2nc(O)n(Cc3ccc(C(=O)NCCCOCCOCCOCCCNC(=O)CCC(=O)Oc4ccc(/C=C/C(=O)NCC(=O)N[C@@H](Cc5ccccc5)C(=O)N[C@H](CCC(=O)OCC)C(=O)OCC)cc4OC)cc3)c2n1]

Diethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3- methoxyphenyl)acryloyl)glycyl-L-phenylalanyl-D-glutamate: To an ice-chilled stirring solution of Intermediate 60 (41 mg, 0.062 mmol, 1.2 eq) in DMF (2 mL) were added DIPEA (27 μL, 0.15 mmol, 3 eq), Intermediate 73 (30 mg, 0.051 mmol, 1 eq) and COMU (26 mg, 0.062 mmol, 1.2 eq). The resulting mixture was stirred at rt for 18 h, after which it was diluted with a mixture of DCM and isopropanol (3/1, 30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo. The solid residue was washed twice with diethyl ether to afford the subject compound as an off-white solid (14 mg, yield: 22%). ¹H NMR (400 MHz, DMSO-d₆) δ10.07 (s, 1H), 8.51-8.36 (m, 2H), 8.33-8.24 (m, 1H), 7.81-7.74 (m, 4H), 7.54-7.30 (m, 5H), 7.29-7.07 (m, 6H), 6.81 (d, 1H), 6.51 (s, 2H), 4.90 (s, 2H), 4.30-4.01 (m, 8H), 3.96 (s, 3H), 3.87 (d, 2H), 3.57-3.37 (m, 10H), 3.33-3.19 (m, 4H), 3.15-3.03 (m, 4H), 2.48-2.40 (m, 4H), 2.23 (t, 2H), 2.00-1.89 (m, 1H), 1.85-1.68 (m, 3H), 1.68-1.56 (m, 4H), 1.44-1.31 (m, 2H), 1.23-1.15 (m, 6H), 0.96-0.85 (m, 3H).

### Example 16: Compound SG200

### Intermediate 75

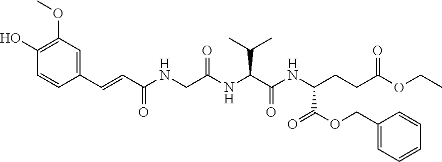 [SMILES: CCOC(=O)CC[C@@H](NC(=O)[C@@H](NC(=O)CNC(=O)/C=C/c1ccc(O)c(OC)c1)C(C)C)C(=O)OCc1ccccc1]

1-benzyl 5-ethyl ((E)-3-(4-hydroxy-3-methoxyphenyl)acryloyl)glycyl-L-yalyl-D-glutamate: Intermediate 42 (156 mg, 0.3 mmol, 1 eq) was added to an ice-chilled stirred mixture of TFA and DCM (1/5, 4 mL), and the mixture was allowed to warm to room temperature. After 3 h, the solvent was evaporated in vacuo. The residue was coevaporated four times with diethyl ether and dissolved in DMF (3 mL). After cooling the solution in ice, DIPEA (0.261 mL, 1.5 mmol, 5 eq), trans-ferulic acid (64 mg, 0.33 mmol, 1.1 eq), HOBt (45 mg, 0.33 mmol, 1.1 eq), DMAP (catalytic amount) and EDC (63 mg, 0.33 mmol, 1.1 eq) were added and the resulting mixture was stirred at rt for 18 h. The reaction mixture was diluted with ethyl acetate (30 mL) and washed with a saturated NH₄Cl solution (2×2 mL), saturated NaHCO₃ solution (2×15 mL) and brine (15 mL). The organic layer was dried over anhydrous Na₂SO₄ and concentrated in vacuo to afford the subject compound as a white solid (89 mg, yield: 50%). ¹H NMR (400 MHz, DMSO-d₆) δ9.45 (s, 1H), 8.45 (d, 1H), 8.19 (t, 1H), 7.91 (d, 1H), 7.41-7.28 (m, 6H), 7.14 (d, 1H), 7.00 (dd, 1H), 6.79 (d, 1H), 6.57 (d, 1H), 5.11 (d, 2H), 4.39-4.31 (m, 1H), 4.30-4.23 (m, 1H), 4.01 (q, 2H), 3.88 (d, 2H), 3.81 (s, 3H), 2.35 (t, 2H), 2.09-1.92 (m, 2H), 1.92-1.79 (m, 1H), 1.14 (t, 3H), 0.83 (t, 6H).

### Compound 76

### SG200

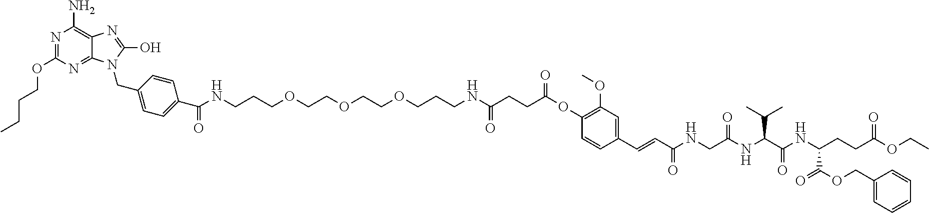 [SMILES: CCCCOc1nc(N)c2nc(O)n(Cc3ccc(C(=O)NCCCOCCOCCOCCCNC(=O)CCC(=O)Oc4ccc(/C=C/C(=O)NCC(=O)N[C@H](C(=O)N[C@H](CCC(=O)OCC)C(=O)OCc5ccccc5)C(C)C)cc4OC)cc3)c2n1]

1-benzyl 5-ethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3-methoxyphenyl)acryloyl)glycyl-L-yalyl-D-glutamate: To an ice-chilled stirring solution of Intermediate 60 (58 mg, 0.088 mmol, 1 eq) in DMF (2 mL) were added DIPEA (47 μL, 0.27 mmol, 3 eq), Intermediate 75 (52 mg, 0.088 mmol, 1 eq) and COMU (42 mg, 0.098 mmol, 1.1 eq). The resulting mixture was stirred at rt for 18 h, after which it was diluted with a mixture of DCM and isopropanol (3/1, 30 mL) and washed with a 1 M HCl solution (2×15 mL), saturated NaHCO₃ solution (2×15 mL) and brine (15 mL). The organic layer was dried over anhydrous Na₂SO₄ and concentrated in vacuo. The solid residue was washed twice with diethyl ether to afford the subject compound as an off-white solid (31 mg, yield: 28%). ¹H NMR (400 MHz, DMSO-d₆) δ9.99 (s, 1H), 8.46 (d, 1H), 8.39 (t, 1H), 8.32 (t, 1H), 7.97 (d, 1H), 7.91 (t, 1H), 7.83-7.74 (m, 2H), 7.47-7.29 (m, 9H), 7.17 (dd, 1H), 7.09 (d, 1H), 6.77 (d, 1H), 6.47 (s, 2H), 5.13 (d, 2H), 4.90 (s, 2H), 4.40-4.24 (m, 2H), 4.12 (t, 2H), 4.01 (q, 2H), 3.91 (d, 2H), 3.80 (s, 3H), 3.54-3.23 (m, 14H), 3.15-3.03 (m, 2H), 2.48-2.41 (m, 4H), 2.38-2.32 (m, 2H), 2.06-1.94 (m, 2H), 1.93-1.81 (m, 1H), 1.78-1.67 (m, 2H), 1.67-1.56 (m, 4H), 1.42-1.30 (m, 2H), 1.14 (t, 3H), 0.93-0.80 (m, 9H).

### Example 17: In vitro Evaluation of Conjugated Compounds

### PBMC Cytotoxicity Assay

The TLR7 and NOD2 conjugated compounds of the invention were tested for their ability to activate the cytotoxic activity of peripheral blood mononuclear cells (PBMCs) towards cancer cell lines. The main effector fraction of PBMCs in this assay are natural killer (NK) cells, which play an essential role in the innate immune system through their direct cytotoxic activity against aberrant cells, especially tumour cells and virally infected cells. The flow cytometry-based method described hereafter measures the PBMC cytotoxic activity against target cancer cell lines by co-incubating PBMCs and cancer cells, which were pre-labelled by carboxyfluorescein succinimidyl ester (CFSE) to distinguish them from effector cells. Successful activation of effector cells by the tested compounds leads to higher degree of cell death in the target cell population.

Chronic myelogenous leukaemia K562 and chronic B cell leukaemia MEC-1 cell lines were used as target cells in the assay. Both cell lines were pre-cultured for at least 10 days before the assay was performed. Immediately prior to the addition of target cells to PBMCs, target cells were stained with CFSE at 2 μM for 15 minutes at 37° C. in the dark. Cells were then washed with medium and resuspended at 200.000 cells/mL.

PBMCs were isolated from whole blood by centrifugation with the Ficoll-Paque density gradient solution. After centrifugation, PBMCs were washed twice with PBS and resuspended in medium at 4.000.000 cells/mL. To perform the assay, PBMCs were seeded on 96 well microtiter plates at 400.000 cells per well and cultured in duplicates at 37° C. for 20 h in the presence of the compounds.

The compounds were prepared as follows: First, stock solutions of the conjugated compounds were prepared in DMSO at a 1 mM concentration. The working dilutions of the conjugated compounds in medium were then added directly to PBMCs to ensure a 1 μM final concentration.

After incubating the PBMCs in the presence of compounds, 10.000 CFSE stained target cells were added to each well for a final 40:1 ratio of effector cells versus target cells. After 4 h of co-incubation, cells were stained with the SYTOXTM Blue (Invitrogen) nucleic acid stain and analysed with flow cytometry. Dead (SYTOXTM Blue stain positive) target cells were gated out of the CFSE positive population, providing the ratio of killed cells within the target cell population.

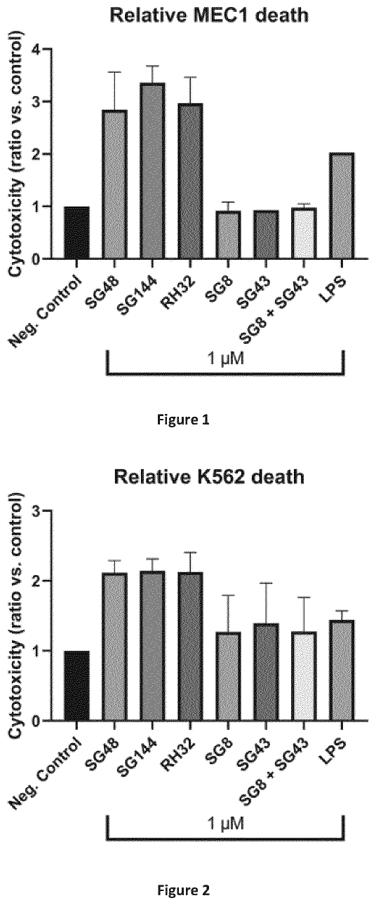

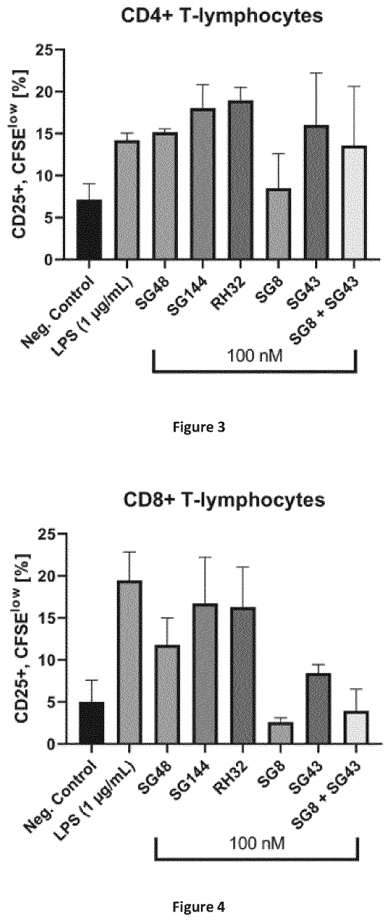

The ability of conjugated compounds to activate the cytotoxic activity of PBMCs against MEC-1 and K562 target cells is demonstrated in FIG. 1 and FIG. 2. SG48, SG144, and RH32 were the tested conjugated compounds of the invention. Medium was used as a control and the results are given as a ratio versus the control. Intermediary compound SG8 was employed as a known NOD2 agonist of the desmuramylpeptide type (Gobec et al. 2018). Intermediary compound SG43 was employed as a known TLR7 agonist of the purine type (Akinbobuyi et al. 2016). The combination of SG8 and SG43 was employed to compare the activity of conjugated compounds against an unconjugated mixture of NOD2 and TLR7 agonists.

### Antigen Presentation of BMDCs to CD4+ and CD8+ T-Lymphocytes

The effect of the conjugated compounds of the invention on antigen presentation of mouse bone marrow derived dendritic cells (BMDCs) to T-lymphocytes was examined. After pre-treating BMDCs with selected compounds and ovalbumin, antigen presentation of ovalbumin to either CD4+ or CD8+ T-lymphocytes was observed with flow cytometry by detecting the degree of activation and proliferation of affected T-lymphocytes.

BMDCs were obtained by harvesting bone marrow from mouse hind legs. The harvested cells were then cultured for 10 days in the presence of granulocyte-macrophage colony-stimulating factor

(GM-CSF) to ensure differentiation into dendritic cells. BMDCs were seeded on 96 well microtiter plates at 10.000 cells per well and stimulated in duplicates with conjugated compounds and ovalbumin (50 μg/mL) at 37° C. for 20 h. After this period, the supernatants were removed and either CD4+ T-Iymphocytes or CD8+ T-Iymphocytes were added.

The compounds were prepared as follows: First, stock solutions of the conjugated compounds were prepared in DMSO at a 1 mM concentration. The working dilutions of the conjugated compounds in medium were then added directly to BMDCs to ensure final concentrations ranging from 1 nM to 1 μM.

CD4+ and CD8+ T-lymphocytes were obtained from single-cell suspensions of spleens from OT-II and OT-I transgenic mice, respectively. These express a T-cell receptor that pairs with the CD4 or CD8 co-receptors and is specific for the ovalbumin antigen. After harvesting the spleen, CD4+ or CD8+ cell fractions were enriched with MACS separation technology (Miltenyi Biotec CD4+ T Cell Isolation Kit or CD8+ T Cell Isolation Kit, respectively). T-lymphocytes were then stained with CFSE and added to BMDCs at 50.000 cells per well.

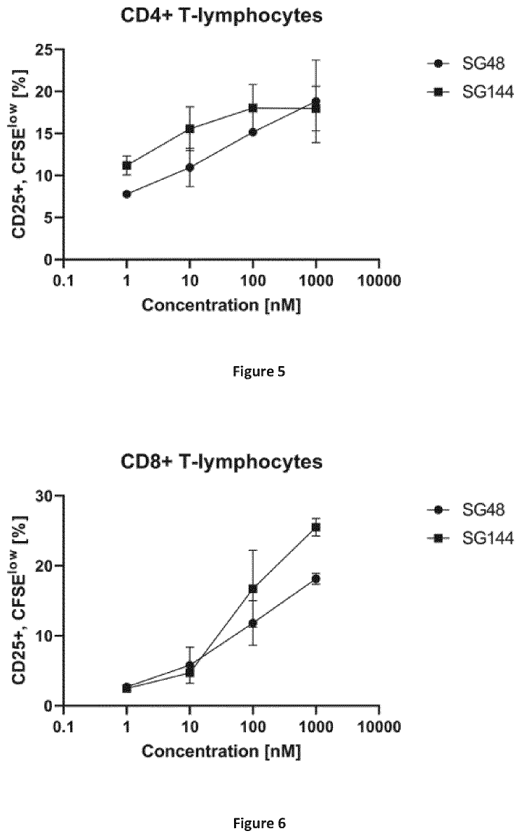

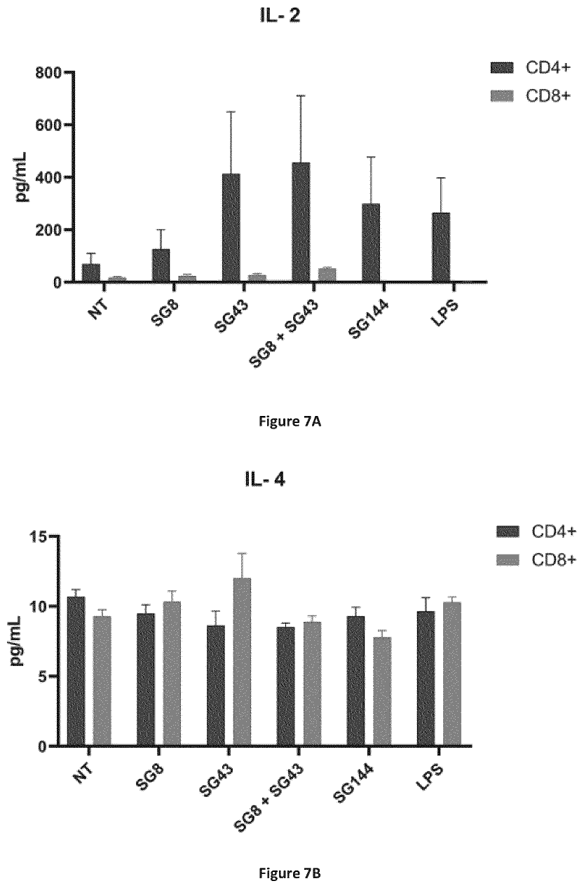

After co-culturing the BMDCs and T-lymphocytes for 72 hours at 37° C., T-lymphocyte activation was assessed with flow cytometry by measuring the expression of CD25, characteristic for activated T-lymphocytes. Concurrently, the intensity of CFSE fluorescence was measured to evaluate T-lymphocyte proliferation. FIG. 3 and FIG. 4 demonstrate the ratio of CD25 positive T-lymphocytes with low CFSE fluorescence intensity, signifying activated T-lymphocytes that have divided in the co-culturing period. SG48, SG144 and RH32 were the tested conjugated compounds of the invention. Medium was used as a negative control. LPS (1 μg/mL) was used as the positive control. Intermediary compound SG8 was employed as a known NOD2 agonist of the desmuramylpeptide type (Gobec et al. 2018). Intermediary compound SG43 was employed as a known TLR7 agonist of the purine type (Akinbobuyi et al. 2016). The combination of SG8 and SG43 was employed to compare the activity of conjugated compounds against an unconjugated mixture of NOD2 and TLR7 agonists.

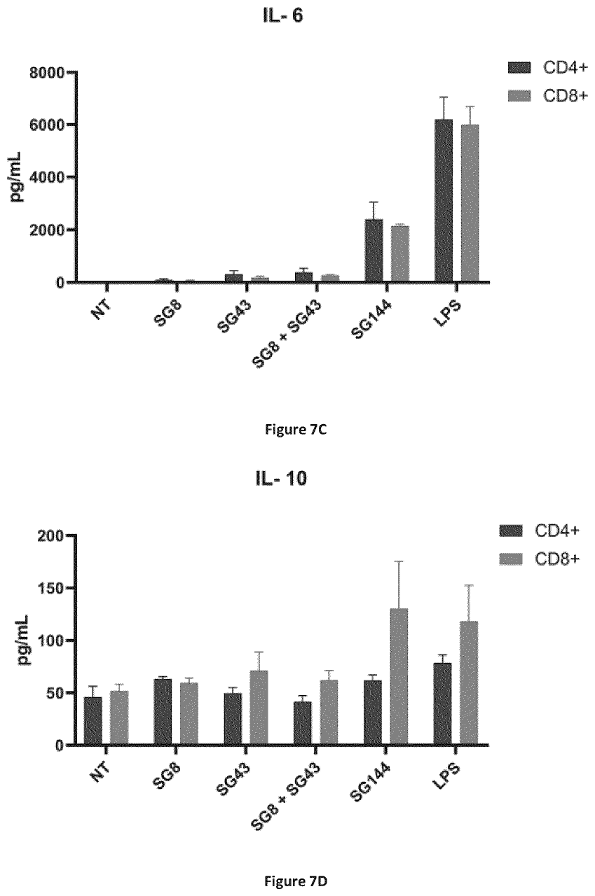

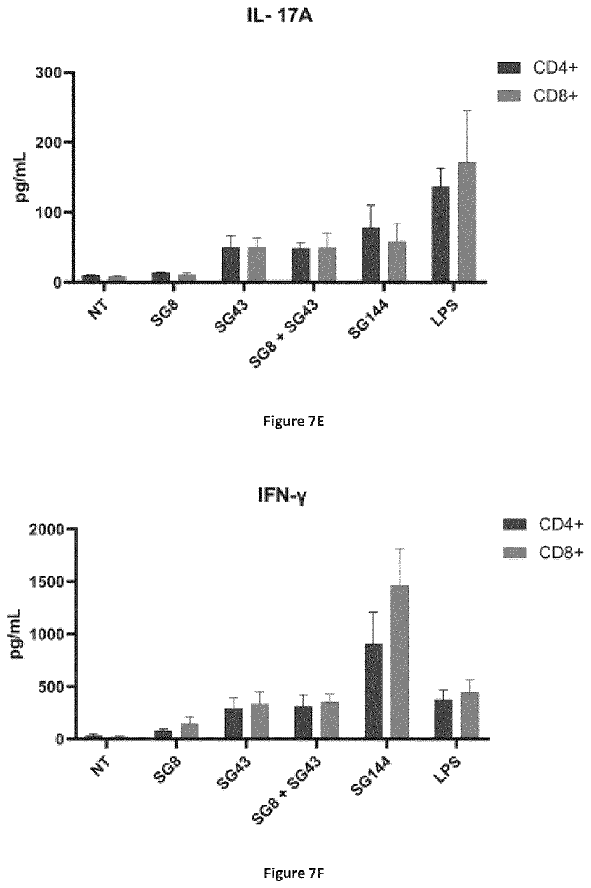

FIG. 5 and FIG. 6 demonstrate the dose-dependent ratios of CD25 positive and CFSE low T-lymphocytes for experiments with compounds SG48 and SG144.

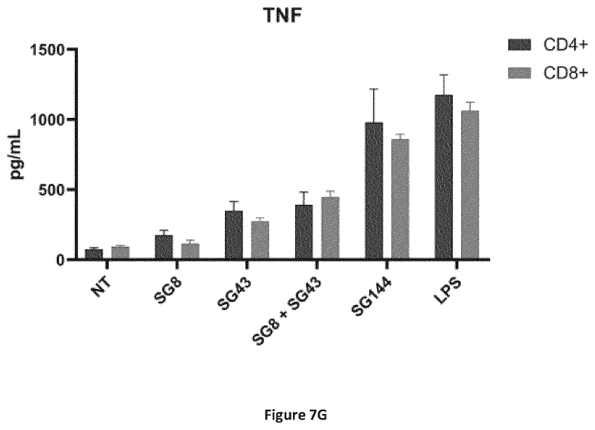

Additionally, the concentrations of selected cytokines were measured in the cell supernatants with the Th1/Th2/Th17 Cytokine Cytometric Bead Array (BD Biosciences). FIGS. 7A-7G demonstrate the CD4+ and CD8+ T-lymphocyte secretion secretion of IL-2 (FIG. 7A), IL-4 (FIG. 7B) , IL-6 (FIG. 7C), IL-10 (FIG. 7D) , IL-17A (FIG. 7E) , IFN-γ (FIG. 7F) and TNF (FIG. 7G) in response to ovalbumin presentation by BMDCs pretreated with compound of the invention (SG144). Medium was used as a negative control. LPS (1 μg/mL) was used as the positive control. Intermediary compound SG8 was employed as a known NOD2 agonist of the desmuramylpeptide type (Gobec et al. 2018). Intermediary compound SG43 was employed as a known TLR7 agonist of the purine type (Akinbobuyi et al. 2016). The combination of SG8 and SG43 was employed to compare the activity of conjugated compounds against an unconjugated mixture of NOD2 and TLR7 agonists.

### References Cited in the Description

Patent Documents Cited in the Description
WO2006/117670, Jones
WO2007/024707, Carson
WO2008/004948, Cook
WO2010/093436, Carson
WO2010/018134, Bazin-Lee
WO2011/134668, Maj
WO2012/038058, Holldack
WO2019/209811, Purandare
WO2019/035969, Young
WO2019/036023, Poudel
WO2019/035971, He
WO2019/197598, Bazin-Lee

Non-Patent Literature Cited in the Description
AKINBOBUYI, B. et al. Facile syntheses of functionalized toll-like receptor 7 agonists. Tetrahedron Lett. 2015. vol. 56,458-460.
AKINBOBUYI, B. et al. Synthesis and immunostimulatory activity of substituted TLR7 agonists. Bioorganic Med. Chem. Lett. 2016. vol. 26,4246-4249.
ASANO, J. et al. Nucleotide Oligomerization Binding Domain-Like Receptor Signaling Enhances Dendritic Cell-Mediated Cross-Priming In Vivo. J. Immunol. 2010. vol. 184,736-745.
BUMGARDNER, S.A. et al. Nod2 is required for antigen-specific humoral responses against antigens orally delivered using a recombinant Lactobacillus vaccine platform. PLoS One 2018. vol. 13, 1-23.
COONEY, R. et al. NOD2 stimulation induces autophagy in dendritic cells influencing bacterial handling and antigen presentation. Nat. Med. 2010. vol. 16,90-97.
GOBEC, M. et al. Structural requirements of acylated Gly-1-Ala-d-Glu analogs for activation of the innate immune receptor NOD2. Eur. J. Med. Chem. 2016. vol. 116,1-12.
GOBEC, M. et al. Discovery of nanomolar desmuramylpeptide agonists of the innate immune receptor nucleotide-binding oligomerization domain-containing protein 2 (NOD2) possessing immunostimulatory properties. J. Med. Chem. 2018. vol. 61,2707-2724.
GUTJAHR, A. et al. Cutting Edge: A Dual TLR2 and TLR7 Ligand Induces Highly Potent Humoral and Cell-Mediated Immune Responses. J. Immunol. 2017. vol. 198,4205-4209.
GUTJAHR, A. et al. Triggering Intracellular Receptors for Vaccine Adjuvantation. Trends Immunol. 2016. vol. 37,573-587.
HEMMI, H. et al. Small-antiviral compounds activate immune cells via the TLR7 MyD88-dependent signaling pathway. Nat. Immunol. 2002. vol. 3,196-200.
JACKSON, E. M. et al. Intranasal vaccination with murabutide enhances humoral and mucosal immune responses to a virus-like particle vaccine. PLoS One 2012. vol. 7, .
JAKOPIN, Ž. Nucleotide-binding oligomerization domain (NOD) inhibitors: A rational approach toward inhibition of NOD signaling pathway. J. Med. Chem. 2014. vol. 57,6897-6918.
JAKOPIN, Ž. et al. Immunomodulatory properties of novel nucleotide oligomerization domain 2 (Nod2) agonistic desmuramyldipeptides. J. Med. Chem. 2012. vol. 55,6478-6488.
JONES, P. et al. Discovery of a highly potent series of TLR7 agonists. Bioorganic Med. Chem. Lett. 2011. vol. 21,5939-5943.
KOBAYASHI, K. S. et al. Nod2-dependent regulation of innate and adaptive immunity in the intestinal tract. Science (80-.). 2005. vol. 307,731-734.
KOBOLD, S. et al. Modes of action of TLR7 agonists in cancer therapy. Immunotherapy 2014. vol. 6, 1085-1095.
KURIMOTO, A. et al. Synthesis and biological evaluation of 8-oxoadenine derivatives as toll-like receptor 7 agonists introducing the antedrug concept. J. Med. Chem. 2010. vol. 53,2964-2972.
KURIMOTO, A. et al. Synthesis and evaluation of 2-substituted 8-hydroxyadenines as potent interferon inducers with improved oral bioavailabilities. Bioorganic Med. Chem. 2004. vol. 12, 1091-1099.
LEE, J. et al. Molecular basis for the immunostimulatory activity of guanine nucleoside analogs: Activation of toll-like receptor 7. Proc. Natl. Acad. Sci. U. S. A. 2003. vol. 100,6646-6651.
MADAN-LALA, R. et al. Combinatorial Delivery of Dual and Triple TLR Agonists via Polymeric Pathogen-like Particles Synergistically Enhances Innate and Adaptive Immune Responses. Sci. Rep. 2017. vol. 7,14-16.
MANCINI, R.J. et al. Covalently Coupled Immunostimulant Heterodimers. Angew. Chemie-Int. Ed. 2014. vol. 53,189-192.
MORRIS, S. et al. Autophagy-Mediated Dendritic Cell Activation Is Essential for Innate Cytokine Production and APC Function with Respiratory Syncytial Virus Responses. J. Immunol. 2011. vol. 187,3953-3961.
NABERGOJ, S. et al. Harnessing the untapped potential of nucleotide-binding oligomerization domain ligands for cancer immunotherapy. Med. Res. Rev. 2019. vol. 39,1447-1484.
NAKAMURA, T. et al. Synthesis and evaluation of 8-oxoadenine derivatives as potent Toll-like receptor 7 agonists with high water solubility. Bioorganic Med. Chem. Lett. 2013. vol. 23,669-672.
PAVOT, V. et al. Cutting Edge: New Chimeric NOD2/TLR2 Adjuvant Drastically Increases Vaccine Immunogenicity. J. Immunol. 2014. vol. 193,5781-5785.
THAISS, C. A. et al. Integration of Innate Immune Signaling. Trends Immunol. 2016. vol. 37,84-101.
TOM, J .K. et al. Applications of Immunomodulatory Immune Synergies to Adjuvant Discovery and Vaccine Development. Trends Biotechnol. 2019. vol. 37,373-388.
TRAN, T. D. et al. Design and optimisation of orally active TLR7 agonists for the treatment of hepatitis C virus infection. Bioorganic Med. Chem. Lett. 2011. vol. 21,2389-2393.
TRAVASSOS, L. H. et al. Nod1 and Nod2 direct autophagy by recruiting ATG16L1 to the plasma membrane at the site of bacterial entry. Nat. Immunol. 2010. vol. 11,55-62.
VIDAL, V. et al. Enhanced maturation and functional capacity of monocyte-derived immature dendritic cells by the synthetic immunomodulator Murabutide. Immunology 2001. vol. 103, 479-487.

1. A compound having a structure according to [symbol] : 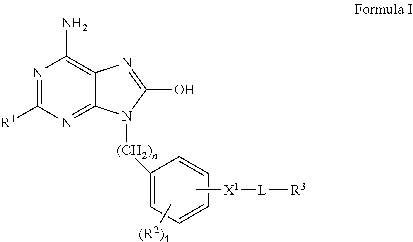
or a pharmaceutically acceptable salt, racemate, diastereomer, enantiomer, ester or prodrug thereof, wherein:
R¹ is H, halogen, OH, SH, CF₃, C₁-C₆ alkyl, C₃-C₁₀ cycloalkyl, C₆-C₁₀ aryl, C₅-C₉ heterocyclyl, C₁-C₆ alkoxy, C₁-C₆ alkoxy C₁-C₆ alkyl, C₁-C₆ alkoxy-C₁-C₆ alkoxy, C₁-C₆ alkoxy-(C₁-C₆ alkyl)S—, (C₁-C₆ alkyl)SO₂NH—, (C₁-C₆ alkyl)C(═O)O—, (C₁-C₆ alkyl)OC(═O)—, (C₁-C₆ alkyl)C(═O)—, (C₁-C₆ alkyl)C(═O)NH—, R^{a}R^{b}N— or R^{a}R^{b}N(C═O)—, wherein alkyl, alkoxy, cycloalkyl, aryl and heterocyclyl may be optionally substituted;
R² is independently for each occurrence selected from H, halogen, OH, CHF₂, CF₃, CH₂CF₂, carboxy, CN, NO₂, C₁-C₆ alkyl, C₃-C₁₀ cycloalkyl, C₁-C₆ alkoxy, (C₁-C₆ alkyl)C(═O)—, (C₆-C₁₀ aryl)C(═O)—, (C₁-C₆ alkyl)S—, (C₁-C₆ alkyl)C(═O)O—, (C₁-C₆ alkyl)OC(═O)—, (C₁-C₆ alkyl)C(═O)NH—, (C₁-C₆ alkyl)SO₂NH—, R^{a}R^{b}N— and R^{a}R^{b}N(C═O)—, wherein alkyl, alkoxy, aryl and cycloalkyl may be optionally substituted;
R^{a} and R^{b} are independently from each other selected from H, C₃-C₁₀ cycloalkyl, C₆-C₁₀ aryl and C₅-C₉ heterocyclyl or R^{a} and R^{b} may together with the nitrogen atom form a C₅-C₆ heterocycle, wherein alkyl, cycloalkyl, aryl and heterocyclyl may be optionally substituted;
X¹ is a single bond, —O—, —S—, —C(═O)— or —SO₂—;
R^{c} is H, C₁-C₆ alkyl or C₃-C₁₀ cycloalkyl;
L is a linking group;
n is 0, 1, 2, 3 or 4; and
R³ is a NOD2 agonist selected from the group consisting of: 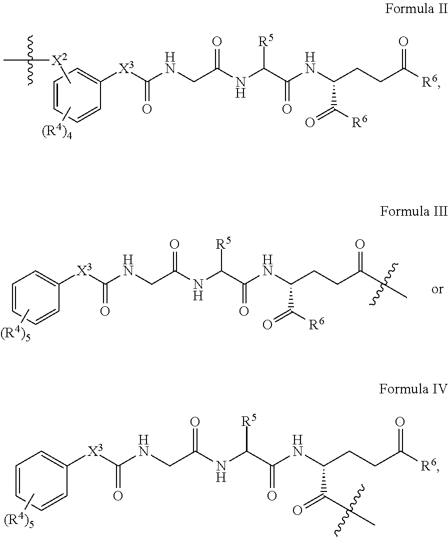
wherein:
X² is a single bond, —O—, —S—, —NR^{d}-, —C(═O)— or —SO₂—;
R^{d} is H, C₁-C₆ alkyl or C₃-C₁₀ cycloalkyl;
X³ is —(CH₂)₂—, —(CH═CH)— or cyclopropylene;
R⁴ is independently for each occurrence selected from H, halogen, OH, CHF₂, CF₃, CH₂CF₂, carboxy, CN, NO₂, C₁-C₆ alkyl, C₁-C₆ alkoxy, C₃-C₁₀ cycloalkyl, (C₁-C₆ alkyl)C(═O)—, (C₁-C₆ aryl)C(═O)—, (C₁-C₆ alkyl)S—, (C₁-C₆ alkyl)C(═O)O—, (C₁-C₆ alkyl)OC(═O)—, (C₁-C₆ alkyl)C(═O)NH—, (C₁-C₆ alkyl)SO₂NH—, R^{e}R^{f}N— and R^{e}R^{f}N(C═O)—, wherein alkyl, alkoxy, aryl and cycloalkyl may be optionally substituted;
R^{e} and R^{f} are independently from each other selected from H, C₁-C₆ alkyl, C₃-C₁₀ cycloalkyl, C₆-C₁₀ aryl and C₅-C₉ heterocyclyl or R^{e} and R^{f} may together with the nitrogen atom form a C₅-C₆ heterocycle;
R⁵ is C₁-C₆ alkyl or a specific side chain of an amino acid;
R⁶ is independently for each occurrence selected from OH, C₁-C₁₈ alkoxy, (C₂-C₁₈ alkenyl)O—, (C₃-C₁₀ cycloalkyl)O—, (C₆-C₁₀ aryl)O—, (C₅-C₉ heterocyclyl)O— and R^{g}R^{h}N—, wherein alkoxy, alkenyl, cycloalkyl, aryl or heterocyclyl may be optionally substituted; and
R^{g} and Rh are independently from each other selected from H, C₁-C₁₈ alkyl, C₂-C₁₈ alkenyl, C₃-C₁₀ cycloalkyl, C₆-C₁₀ aryl and C₅-C₉ heterocyclyl or R^{g} and R^{h} may together with the nitrogen atom form a C₅-C₆ heterocycle wherein alkyl, alkenyl, cycloalkyl, aryl and heterocyclyl may be optionally substituted.

2. The compound according to claim 1, wherein n is 1.

3. The compound according to claim 1, wherein R¹ is hydrogen, C₁-C₆ alkoxy, C₁-C₆ alkoxy-C₁-C₆ alkoxy, (C₁-C₆ alkyl)S— or CF₃.

4. (canceled)

5. The compound according to claim 1, wherein R¹ is n-BuO—.

6. The compound according to claim 1, wherein R² is independently for each instance selected from hydrogen, halogen and C₁-C₆ alkyl.

7. (canceled)

8. The compound according to claim 1, wherein X¹ is —O—, —NH— or —C(═O)—.

9. The compound according to claim 1, wherein L is selected from the group consisting of an amino acid, a peptide, a non-peptidic polymeric linker and a non-polymeric aliphatic linker.

10. (canceled)

11. The compound according to claim 10, wherein Lisa polyethylene glycol chain comprising of 2 to 100 repeating ethylene glycol units.

12. (canceled)

13. (canceled)

14. The compound according to claim 1, wherein X² is —O—, —NH— or —C(═O)—.

15. The compound according to claim 1, wherein X³ is —CH═CH— or cyclopropylene.

16. The compound according to claim 1, wherein R⁴ is independently for each instance selected from hydrogen, halogen, OH, C₁-C₆ alkyl and C₁-C₆ alkoxy.

17. The compound according to claim 1, wherein R⁵ is C₁-C₆ alkyl or a specific side chain of a natural amino acid.

18. The compound according to claim 17, wherein R⁵ is C₁-C₆ alkyl or the specific side chain of valine, alanine, phenylalanine, leucine or isoleucine.

19. (canceled)

20. The compound according to claim 1, wherein R⁶ is independently for each instance selected from OH, NH₂, (C₂-C₁₈ alkenyl)O—, (C₃-C₁₀ cycloalkyl)O— and C₁-C₁₈ alkoxy.

21. (canceled)

22. The compound according to claim 1, wherein said compound is selected from the group consisting of:
Diethyl ((E)-3-(4-((6-(4-((6-amino-2-butoxy-8-hydroxy-9H-purin-9-yl)methyl)benzamido)hexanoyl)-oxy)-3-methoxyphenyl)acryloyl)glycyl-L-valyl-D-glutamate,
Ethyl N5-(2-(2-(4-((6-amino-2-butoxy-8-hydroxy-9H-purin-9-yl)methyl)benzamido)ethoxy)ethyl)-N2-((E)-3-(4-hydroxy-3-methoxyphenyl)acryloyl)glycyl-L- valyl-D-glutaminate,
Ethyl (R)-1-(4-((6-amino-2-butoxy-8-hydroxy-9H-purin-9-yl)methyl)phenyl)-15-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate,
Ethyl (R)-1-(4-((6-amino-2-butoxy-8-hydroxy-9H-purin-9-yl)methyl)phenyl)-15-((S)-2-(2-((E)-3-(4-isopropylphenyl)acrylamido)acetamido)-3- methylbutanamido)-1,12-dioxo-5,8-dioxa-2,11-diazahexadecan-16-oate,
Ethyl (R)-1-(4-((6-amino-2-butoxy-8-hydroxy-9H-purin-9-yl)methyl)phenyl)-15-((S)-2-(2-((E)-3-(3,4-difluorophenyl)acrylamido)acetamido)-3- methylbutanamido)-1,12-dioxo-5,8-dioxa-2,11-diazahexadecan-16-oate,
Ethyl (15R)-1-(4-((6-amino-2-butoxy-8-hydroxy-9H-purin-9-yl)methyl)phenyl)-methylbutanamido)-1,12-dioxo-5,8-dioxa-2,11-diazahexadecan-16-oate,
Ethyl (R)-1-(4-((6-amino-2-butoxy-8-hydroxy-9H-purin-9-yl)methyl)phenyl)-13-((S)-2-(2-((E)-3-(4-hydroxy-3-methoxyphenyl)acrylamido)acetamido)-3-methylbutanamido)-1,12-dioxo-5,8-dioxa-2,11-diazahexadecan-16-oate,
Ethyl (R)-1-(4-((6-amino-2-butoxy-8-hydroxy-9H-purin-9-yl)methyl)phenyl)-13-((S)-2-(2-cinnamamidoacetamido)-3-methylbutanamido)-1,12-dioxo-5,8- dioxa-2,11-diazahexadecan-16-oate,
Ethyl (13R)-1-(4-((6-amino-2-butoxy-8-hydroxy-9H-purin-9-yl)methyl)phenyl)-13-((2S)-2-(2-(2-(3,4-difluorophenyl)cyclopropane-1- carboxamido)acetamido)-3-methylbutanamido)-1,12-dioxo-5,8-dioxa-2,11-diazahexadecan-16-oate,
Diethyl ((E)-3-(4-(1-(4-((6-amino-2-butoxy-8-hydroxy-9H-purin-9-yl)methyl)phenyl)-1,17-dioxo-6,9,12-trioxa-2,16-diazaicosan-20- amido)phenyl)acryloyl)glycyl-L-valyl-D-glutamate,
Dicyclopentyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3-methoxyphenyl)acryloyl)glycyl-L-valyl-D-glutamate,
Diethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3- methoxyphenyl)acryloyl)glycyl-L-alanyl-D-glutamate,
Diethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3- methoxyphenyl)acryloyl)glycyl-L-phenylalanyl-D-glutamate, and
1-benzyl 5-ethyl ((E)-3-(4-((1-(4-((6-amino-2-butoxy-8-hydroxy-9H-purin-9-yl)methyl)phenyl)-1,17-dioxo-6,9,12-trioxa-2,16-diazaicosan-20-oyl)oxy)-3-methoxyphenyl)acryloyl)glycyl-L-valyl-D-glutamate.

23. A process for preparing a compound of Formula I as defined in claim 1 (with the variable groups being as defined in any one of claims 1 to 22) which comprises reacting a compound of [symbol] : 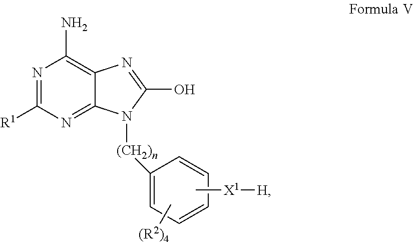
with a compound of [symbol] :
H—L—R³ Formula VI,
or a compound of [symbol] : 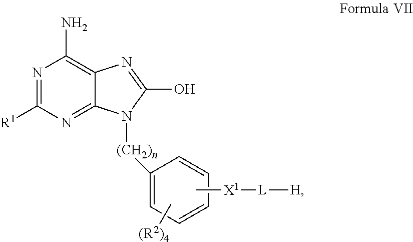
with a compound of [symbol] : 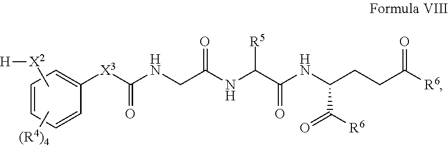 [symbol]
: 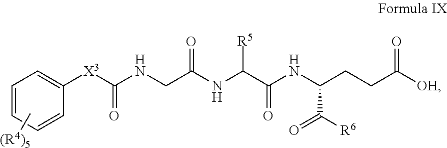
or [symbol] : 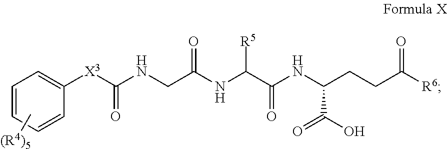
and optionally thereafter carrying out one or more of the following procedures:
removing any protecting groups,
forming a pharmaceutically acceptable salt,
converting a compound of Formula I into another compound of Formula I.

24. (canceled)

25. (canceled)

26. The method of claim 29, wherein the condition is selected from the group consisting of viral infections, bacterial infections, fungal infections, protozoal infections, tumors, cancers and immunological diseases

27. A pharmaceutical composition comprising the compound according to claim 1 and one or more pharmaceutically acceptable excipients or carriers.

28. A vaccine comprising a compound according to claim 1.

29. A method of treating a condition in which agonism of TLR7 and NOD2 receptors is beneficial, comprising administering the compound according to claim 1 to a subject in need thereof.
